(19) United States
(12) Patent Application Publication
(10) Pub. No.: US 2010/0099083 A1
(43) Pub. Date: Apr. 22, 2010

(54) CROHN DISEASE SUSCEPTIBILITY GENE

(75) Inventors: John Verner Raelson, Ville St-Laurent (CA); Stefan Schreiber, Ville St-Laurent (CA); Randall David Little, Ville St-Laurent (CA); Andre Franke, Ville St-Laurent (CA); Jochen Hampe, Ville St-Laurent (CA); Tim Keith, Ville St-Laurent (CA); Vanessa Bruat, Ville St-Laurent (CA); Abdelmajid Belouchi, Ville St-Laurent (CA)

Correspondence Address: DOWELL & DOWELL P.C., 103 Oronoco St., Suite 220, Alexandria, VA, 22314, US

(73) Assignee: GENIZON BIOSCIENCES INC, VILLE ST-LAURENT (CA)

(21) Appl. No.: 12/309,635

(22) PCT Filed: Jul. 26, 2007
(86) PCT No.: PCT/US07/74481

§ 371 Date: Nov. 30, 2009

Related U.S. Application Data
(60) Provisional application No. 60/833,261, filed on Jul. 26, 2006.
(60) Provisional application No. 60/834,151, filed on Jul. 31, 2006.

(51) Int. Cl. C12Q 1/68; G01N 33/53; C40B 20/00

(52) U.S. Cl. 435/6; 436/94; 435/7.21; 506/2

(57) ABSTRACT

The present invention relates to the ATG16l1 gene and genetic variants associated with Crohn's disease. In particular, the invention relates to the fields of pharmacogenomics, diagnostics, patient therapy and the use of genetic haplotype information to predict an individual's susceptibility to Crohn's disease and/or their response to a particular drug or drugs.

### CROSS-REFERENCE TO RELATED APPLICATIONS

This application claims priority to U.S. Provisional Application No. 60/833,261, filed Jul. 26, 2006 and U.S. Provisional Application No. 60/834,151, filed Jul. 31, 2006, which are herein incorporated by reference in their entirety.

The contents of the Jul. 31, 2006 submission on compact discs are incorporated herein by reference in their entirety: A compact disc copy of the Sequence Listing (COPY 1) (filename: GENI 018 01US SeqList.txt, date recorded: Jul. 31, 2006, file size 793,000 bytes); a duplicate compact disc copy of the Sequence Listing (COPY 2) (filename: GENI 018 01US SeqList.txt, date recorded: Jul. 31, 2006, file size 793,000 bytes); a computer readable format copy of the Sequence Listing (CRF COPY) (filename: GENI 018 01US SeqList.txt, date recorded: Jul. 31, 2006, file size 793,000 bytes).

### FIELD OF THE INVENTION

The invention relates to the field of genomics and genetics, including genome analysis and the study of DNA variations. In particular, the invention relates to the fields of pharmacogenomics, diagnostics, patient therapy and the use of genetic haplotype information to predict an individual's susceptibility to Crohn's disease and/or their response to a particular drug or drugs, so that drugs tailored to genetic differences of population groups may be developed and/or administered to the appropriate population.

The invention also relates to the autophagy-related 16-like (ATG16L1) gene for Crohn's disease, which links variations in DNA (including both genic and non-genic regions) to an individual's susceptibility to Crohn's disease and/or response to a particular drug or drugs.

The invention further relates to the genes disclosed in the Crohn Disease candidate region 1 (see Tables 4-6) and, which is related to methods and reagents for detection of an individual's increased or decreased risk for Crohn's disease by identifying at least one polymorphism in one or a combination of the genes from candidate region 1, which are associated with Crohn's disease.

In addition, the invention further relates to nucleotide sequences of those genes including genomic DNA sequences, cDNA sequences, single nucleotide polymorphisms (SNPs), other types of polymorphisms (insertions, deletions, microsatellites) found in candidate regon 1, alleles and haplotypes (see Sequence Listing and Tables 2, 3 and 7-10).

The invention further relates to isolated nucleic acids comprising these nucleotide sequences and isolated polypeptides or peptides encoded thereby. Also related are expression vectors and host cells comprising the disclosed nucleic acids or fragments thereof, as well as antibodies that bind to the encoded polypeptides or peptides.

The present invention further relates to ligands that modulate the activity of the disclosed genes or gene products. In addition, the invention relates to diagnostics and therapeutics for Crohn's disease, utilizing the disclosed nucleic acids, polymorphisms, chromosomal regions, gene maps, polypeptides or peptides, antibodies and/or ligands and small molecules that activate or repress relevant signaling events.

### BACKGROUND OF THE INVENTION

Inflammatory bowel disease has a prevalence of 2-5/1000 individuals in West-European and North-American populations, with a median age of onset in early adulthood. The disease is characterized by chronic relapsing intestinal mucosal inflammation, leading to abdominal pain, chronic diarrhoea, rectal bleeding, weight loss and different intestinal and extra-intestinal manifestations including arthritis and uveitis. On the basis of clinical and histopathological features, IBD can be categorized into two main subtypes, Crohn disease and ulcerative colitis. A genetic component in the aetiology of IBD has been demonstrated by both epidemiological and molecular genetic studies. Thus, epidemiological investigations have consistently revealed familial clustering of the disease and an increased concordance of the IBD phenotype in monozygotic twins. Family data further suggest that the genetic contribution to Crohn disease is greater than that to UC, with relative sibling risk estimates (λ_{S}) ranging from 15 to 35 for Crohn disease and from 6 to 9 for UC, depending upon the population and ascertainment method used.

Crohn's disease is an Inflammatory Bowel Disease (IBD) in which inflammation extends beyond the inner gut lining and penetrates deeper layers of the intestinal wall of any part of the digestive system (esophagus, stomach, small intestine, large intestine, and/or anus). Crohn's disease is a chronic, lifelong disease which can cause painful, often life altering symptoms including diarrhea, cramping and rectal bleeding. Crohn's disease occurs most frequently in the industrialized world and the typical age of onset falls into two distinct ranges, 15 to 30 years of age and 60 to 80 years of age. The highest mortality is during the first years of disease, and in cases where the disease symptoms are long lasting, an increased risk of colon cancer is observed. Crohn's disease presently accounts for approximately two thirds of IBD-related physician visits and hospitalizations, and 50 to 80% of Crohn's disease patients eventually require surgical treatment. Development of Crohn's disease is influenced by environmental and host specific factors, together with “exogenous biological factors” such as constituents of the intestinal flora (the naturally occurring bacteria found in the intestine). It is believed that in genetically predisposed individuals, exogenous factors such as infectious agents, and host-specific characteristics such as intestinal barrier function and/or blood supply, combine with specific environmental factors to cause a chronic state of improperly regulated immune system function. In this hypothetical model, microorganisms trigger an immune response in the intestine, and in susceptible individuals, this immune response is not turned off when the microorganism is cleared from the body. The chronically “turned on” immune response causes damage to the intestine resulting in the symptoms of Crohn's disease.

Current treatments for Crohn's disease are primarily aimed at reducing symptoms by suppressing inflammation and do not address the root cause of the disease. Despite a preponderance of evidence showing inheritance of a risk for Crohn's disease through epidemiological studies and genome wide linkage analyses, the genes affecting Crohn's disease have yet to be discovered (Hugot J P, and Thomas G., 1998). There is a need in the art for identifying specific genes related to Crohn's disease to enable the development of therapeutics that address the causes of the disease rather than relieving its symptoms. The failure in past studies to identify causative genes in complex diseases, such as Crohn's disease, has been due to the lack of appropriate methods to detect a sufficient number of variations in genomic DNA samples (markers), the insufficient quantity of necessary markers available, and the number of needed individuals to enable such a study. The present invention addresses these issues.

### DEFINITIONS

Throughout the description of the present invention, several terms are used that are specific to the science of this field. For the sake of clarity and to avoid any misunderstanding, these definitions are provided to aid in the understanding of the specification and claims:

Allele: One of a pair, or series, of forms of a gene or non-genic region that occur at a given locus in a chromosome. Alleles are symbolized with the same basic symbol (e.g., B for dominant and b for recessive; B1, B2, Bn for n additive alleles at a locus). In a normal diploid cell there are two alleles of any one gene (one from each parent), which occupy the same relative position (locus) on homologous chromosomes. Within a population there may be more than two alleles of a gene. See multiple alleles. SNPs also have alleles, i.e., the two (or more) nucleotides that characterize the SNP.

Amplification of nucleic acids: refers to methods such as polymerase chain reaction (PCR), ligation amplification (or ligase chain reaction, LCR) and amplification methods based on the use of Q-beta replicase. These methods are well known in the art and are described, for example, in U.S. Pat. Nos. 4,683,195 and 4,683,202. Reagents and hardware for conducting PCR are commercially available. Primers useful for amplifying sequences from the disorder region are preferably complementary to, and preferably hybridize specifically to, sequences in the disorder region or in regions that flank a target region therein. Genes from Tables 4-6 generated by amplification may be sequenced directly. Alternatively, the amplified sequence(s) may be cloned prior to sequence analysis.

Antigenic component: is a moiety that binds to its specific antibody with sufficiently high affinity to form a detectable antigen-antibody complex.

Antibodies: refer to polyclonal and/or monoclonal antibodies and fragments thereof, and immunologic binding equivalents thereof, that can bind to proteins and fragments thereof or to nucleic acid sequences from the disorder region, particularly from the disorder gene products or a portion thereof. The term antibody is used both to refer to a homogeneous molecular entity, or a mixture such as a serum product made up of a plurality of different molecular entities. Proteins may be prepared synthetically in a protein synthesizer and coupled to a carrier molecule and injected over several months into rabbits. Rabbit sera are tested for immunoreactivity to the protein or fragment. Monoclonal antibodies may be made by injecting mice with the proteins, or fragments thereof. Monoclonal antibodies can be screened by ELISA and tested for specific immunoreactivity with protein or fragments thereof (Harlow et al., 1988, Antibodies: A Laboratory Manual, Cold Spring Harbor Laboratory, Cold Spring Harbor, N.Y.). These antibodies will be useful in developing assays as well as therapeutics.

Associated allele: refers to an allele at a polymorphic locus that is associated with a particular phenotype of interest, e.g., a predisposition to a disorder or a particular drug response.

cDNA: refers to complementary or copy DNA produced from an RNA template by the action of RNA-dependent DNA polymerase (reverse transcriptase). Thus, a cDNA clone means a duplex DNA sequence complementary to an RNA molecule of interest, included in a cloning vector or PCR amplified. This term includes genes from which the intervening sequences have been removed.

cDNA library: refers to a collection of recombinant DNA molecules containing cDNA inserts that together comprise essentially all of the expressed genes of an organism or tissue. A cDNA library can be prepared by methods known to one skilled in the an (see, e.g., Cowell and Austin, 1997, “DNA Library Protocols,” Methods in Molecular Biology). Generally, RNA is first isolated from the cells of the desired organism, and the RNA is used to prepare cDNA molecules.

Cloning: refers to the use of recombinant DNA techniques to insert a particular gene or other DNA sequence into a vector molecule. In order to successfully clone a desired gene, it is necessary to use methods for generating DNA fragments, for joining the fragments to vector molecules, for introducing the composite DNA molecule into a host cell in which it can replicate, and for selecting the clone having the target gene from amongst the recipient host cells.

Cloning vector: refers to a plasmid or phage DNA or other DNA molecule that is able to replicate in a host cell. The cloning vector is typically characterized by one or more endonuclease recognition sites at which such DNA sequences may be cleaved in a determinable fashion without loss of an essential biological function of the DNA, and which may contain a selectable marker suitable for use in the identification of cells containing the vector.

Coding sequence or a protein-coding sequence: is a polynucleotide sequence capable of being transcribed into mRNA and/or capable of being translated into a polypeptide or peptide. The boundaries of the coding sequence are typically determined by a translation start codon at the 5-terminus and a translation stop codon at the 3′-terminus.

Complement of a nucleic acid sequence: refers to the antisense sequence that participates in Watson-Crick base-pairing with the original sequence.

Disorder region: refers to the portions of the human chromosome displayed in Table 1 bounded by the markers from Tables 2, 3 and 7-10.

Disorder-associated nucleic acid or polypeptide sequence: refers to a nucleic acid sequence that maps to region of Table 1 or the polypeptides encoded therein (Tables 4-6, nucleic acids, and polypeptides). For nucleic acids, this encompasses sequences that are identical or complementary to the gene sequences from Tables 4-6, as well as sequence-conservative, function-conservative, and non-conservative variants thereof. For polypeptides, this encompasses sequences that are identical to the polypeptide, as well as function-conservative and non-conservative variants thereof. Included are the alleles of naturally-occurring polymorphisms causative of Crohn's disease such as, but not limited to, alleles that cause altered expression of genes of Tables 4-6 and alleles that cause altered protein levels or stability (e.g., decreased levels, increased levels, expression in an inappropriate tissue type, increased stability, and decreased stability).

Expression vector: refers to a vehicle or plasmid that is capable of expressing a gene that has been cloned into it, after transformation or integration in a host cell. The cloned gene is usually placed under the control of (i.e., operably linked to) a regulatory sequence.

Function-conservative variants: are those in which a change in one or more nucleotides in a given codon position results in a polypeptide sequence in which a given amino acid residue in the polypeptide has been replaced by a conservative amino acid substitution. Function-conservative variants also include analogs of a given polypeptide and any polypeptides that have the ability to elicit antibodies specific to a designated polypeptide.

Founder population: Also called a population isolate, this is a large number of people who have mostly descended, in genetic isolation from other populations, from a much smaller number of people who lived many generations ago.

Gene: Refers to a DNA sequence that encodes through its template or messenger RNA a sequence of amino acids characteristic of a specific peptide, polypeptide, or protein. The term “gene” also refers to a DNA sequence that encodes an RNA product. The term gene as used herein with reference to genomic DNA includes intervening, non-coding regions, as well as regulatory regions, and can include 5′ and 3′ ends. A gene sequence is wild-type if such sequence is usually found in individuals unaffected by the disorder or condition of interest. However, environmental factors and other genes can also play an important role in the ultimate determination of the disorder. In the context of complex disorders involving multiple genes (oligogenic disorder), the wild type, or normal sequence can also be associated with a measurable risk or susceptibility, receiving its reference status based on its frequency in the general population.

Genotype: Set of alleles at a specified locus or loci.

Haplotype: The allelic pattern of a group of (usually contiguous) DNA markers or other polymorphic loci along an individual chromosome or double helical DNA segment. Haplotypes identify individual chromosomes or chromosome segments. The presence of shared haplotype patterns among a group of individuals implies that the locus defined by the haplotype has been inherited, identical by descent (IBD), from a common ancestor. Detection of identical by descent haplotypes is the basis of linkage disequilibrium (LD) mapping. Haplotypes are broken down through the generations by recombination and mutation. In some instances, a specific allele or haplotype may be associated with susceptibility to a disorder or condition of interest, e.g., Crohn's disease. In other instances, an allele or haplotype may be associated with a decrease in susceptibility to a disorder or condition of interest, i.e., a protective sequence.

Host: includes prokaryotes and eukaryotes. The term includes an organism or cell that is the recipient of an expression vector (e.g., autonomously replicating or integrating vector).

Hybridizable: nucleic acids are hybridizable to each other when at least one strand of the nucleic acid can anneal to another nucleic acid strand under defined stringency conditions. In some embodiments, hybridization requires that the two nucleic acids contain at least 10 substantially complementary nucleotides; depending on the stringency of hybridization, however, mismatches may be tolerated. The appropriate stringency for hybridizing nucleic acids depends on the length of the nucleic acids and the degree of complementarity, and can be determined in accordance with the methods described herein.

Identity by descent (IBD): Identity among DNA sequences for different individuals that is due to the fact that they have all been inherited from a common ancestor. LD mapping identifies IBD haplotypes as the likely location of disorder genes shared by a group of patients.

Identity: as known in the art, is a relationship between two or more polypeptide sequences or two or more polynucleotide sequences, as determined by comparing the sequences. In the art, identity also means the degree of sequence relatedness between polypeptide or polynucleotide sequences, as the case may be, as determined by the match between strings of such sequences. Identity and similarity can be readily calculated by known methods, including but not limited to those described in A. M. Lesk (ed), 1988, Computational Molecular Biology, Oxford University Press, NY; D. W. Smith (ed), 1993, Biocomputing. Informatics and Genome Projects, Academic Press, NY; A. M. Griffin and H. G. Griffin, H. G (eds), 1994, Computer Analysis of Sequence Data, Part 1, Humana Press, NJ; G. von Heinje, 1987, Sequence Analysis in Molecular Biology, Academic Press; and M. Gribskov and J. Devereux (eds), 1991, Sequence Analysis Primer, M Stockton Press, NY; H. Carillo and D. Lipman, 1988, SIAM J. Applied Math., 48:1073.

Immunogenic component: is a moiety that is capable of eliciting a humoral and/or cellular immune response in a host animal.

Isolated nucleic acids: are nucleic acids separated away from other components (e.g., DNA, RNA, and protein) with which they are associated (e.g., as obtained from cells, chemical synthesis systems, or phage or nucleic acid libraries). Isolated nucleic acids are at least 60% free, preferably 75% free, and most preferably 90% free from other associated components. In accordance with the present invention, isolated nucleic acids can be obtained by methods described herein, or other established methods, including isolation from natural sources (e.g., cells, tissues, or organs), chemical synthesis, recombinant methods, combinations of recombinant and chemical methods; and library screening methods.

Isolated polypeptides or peptides: are those that are separated from other components (e.g., DNA, RNA, and other polypeptides or peptides) with which they are associated (e.g., as obtained from cells, translation systems, or chemical synthesis systems). In a preferred embodiment, isolated polypeptides or peptides are at least 10% pure; more preferably, 80% or 90% pure. Isolated polypeptides and peptides include those obtained by methods described herein, or other established methods, including isolation from natural sources (e.g., cells, tissues, or organs), chemical synthesis, recombinant methods, or combinations of recombinant and chemical methods. Proteins or polypeptides referred to herein as recombinant are proteins or polypeptides produced by the expression of recombinant nucleic acids. A portion as used herein with regard to a protein or polypeptide, refers to fragments of that protein or polypeptide. The fragments can range in size from 5 amino acid residues to all but one residue of the entire protein sequence. Thus, a portion or fragment can be at least 5, 5-50, 50-100, 100-200, 200-400, 400-800, or more consecutive amino acid residues of a protein or polypeptide.

Linkage disequilibrium (LD): the situation in which the alleles for two or more loci do not occur together in individuals sampled from a population at frequencies predicted by the product of their individual allele frequencies. In other words, markers that are in LD do not follow Mendel's second law of independent random segregation. LD can be caused by any of several demographic or population artifacts as well as by the presence of genetic linkage between markers. However, when these artifacts are controlled and eliminated as sources of LD, then LD results directly from the fact that the loci involved are located close to each other on the same chromosome so that specific combinations of alleles for different markers (haplotypes) are inherited together. Markers that are in high LD can be assumed to be located near each other and a marker or haplotype that is in high LD with a genetic trait can be assumed to be located near the gene that affects that trait. The physical proximity of markers can be measured in family studies where it is called linkage or in population studies where it is called linkage disequilibrium.

LD mapping: population based gene mapping, which locates disorder genes by identifying regions of the genome where haplotypes or marker variation patterns are shared statistically more frequently among disorder patients compared to healthy controls. This method is based upon the assumption that many of the patients will have inherited an allele associated with the disorder from a common ancestor (IBD), and that this allele will be in LD with the disorder gene.

Locus: a specific position along a chromosome or DNA sequence. Depending upon context, a locus could be a gene, a marker, a chromosomal band or a specific sequence of one or more nucleotides.

Minor allele frequency (MAF): the population frequency of one of the alleles for a given polymorphism, which is equal or less than 50%. The sum of the MAF and the Major allele frequency equals one.

Markers: an identifiable DNA sequence that is variable (polymorphic) for different individuals within a population. These sequences facilitate the study of inheritance of a trait or a gene. Such markers are used in mapping the order of genes along chromosomes and in following the inheritance of particular genes; genes closely linked to the marker or in LD with the marker will generally be inherited with it. Two types of markers are commonly used in genetic analysis, microsatellites and SNPs.

Microsatellite: DNA of eukaryotic cells comprising a repetitive, short sequence of DNA that is present as tandem repeats and in highly variable copy number, flanked by sequences unique to that locus.

Mutant sequence: if it differs from one or more wild-type sequences. For example, a nucleic acid from a gene listed in Tables 4-6 containing a particular allele of a single nucleotide polymorphism may be a mutant sequence. In some cases, the individual carrying this allele has increased susceptibility toward the disorder or condition of interest. In other cases, the mutant sequence might also refer to an allele that decreases the susceptibility toward a disorder or condition of interest and thus acts in a protective manner. The term mutation may also be used to describe a specific allele of a polymorphic locus.

Non-conservative variants: are those in which a change in one or more nucleotides in a given codon position results in a polypeptide sequence in which a given amino acid residue in a polypeptide has been replaced by a non-conservative amino acid substitution, Non-conservative variants also include polypeptides comprising non-conservative amino acid substitutions.

Nucleic acid or polynucleotide: purine- and pyrimidine-containing polymers of any length, either polyribonucleotides or polydeoxyribonucleotide or mixed polyribo polydeoxyribonucleotides. This includes single- and double-stranded molecules, i.e., DNA-DNA, DNA-RNA and RNA-RNA hybrids, as well as protein nucleic acids (PNA) formed by conjugating bases to an amino acid backbone. This also includes nucleic acids containing modified bases.

Nucleotide: a nucleotide, the unit of a DNA molecule, is composed of a base, a 2′-deoxyribose and phosphate ester(s) attached at the 5′ carbon of the deoxyribose. For its incorporation in DNA, the nucleotide needs to possess three phosphate esters but it is converted into a monoester in the process.

Operably linked: means that the promoter controls the initiation of expression of the gene. A promoter is operably linked to a sequence of proximal DNA if upon introduction into a host cell the promoter determines the transcription of the proximal DNA sequence(s) into one or more species of RNA. A promoter is operably linked to a DNA sequence if the promoter is capable of initiating transcription of that DNA sequence.

Ortholog: denotes a gene or polypeptide obtained from one species that has homology to an analogous gene or polypeptide from a different species.

Paralog: denotes a gene or polypeptide obtained from a given species that has homology to a distinct gene or polypeptide from that same species.

Phenotype: any visible, detectable or otherwise measurable property of an organism such as symptoms of, or susceptibility to, a disorder.

Polymorphism: occurrence of two or more alternative genomic sequences or alleles between or among different genomes or individuals at a single locus. A polymorphic site thus refers specifically to the locus at which the variation occurs. In some cases, an individual carrying a particular allele of a polymorphism has an increased or decreased susceptibility toward a disorder or condition of interest.

Portion and fragment: are synonymous. A portion as used with regard to a nucleic acid or polynucleotide refers to fragments of that nucleic acid or polynucleotide. The fragments can range in size from 8 nucleotides to all but one nucleotide of the entire gene sequence. Preferably, the fragments are at least about 8 to about 10 nucleotides in length; at least about 12 nucleotides in length; at least about 15 to about 20 nucleotides in length; at least about 25 nucleotides in length; or at least about 35 to about 55 nucleotides in length.

Probe or primer: refers to a nucleic acid or oligonucleotide that forms a hybrid structure with a sequence in a target region of a nucleic acid due to complementarity of the probe or primer sequence to at least one portion of the target region sequence.

Protein and polypeptide: are synonymous. Peptides are defined as fragments or portions of polypeptides, preferably fragments or portions having at least one functional activity (e.g., proteolysis, adhesion, fusion, antigenic, or intracellular activity) as the complete polypeptide sequence.

Recombinant nucleic acids: nucleic acids which have been produced by recombinant DNA methodology, including those nucleic acids that are generated by procedures which rely upon a method of artificial replication, such as the polymerase chain reaction (PCR) and/or cloning into a vector using restriction enzymes. Portions of recombinant nucleic acids which code for polypeptides can be identified and isolated by, for example, the method of M. Jasin et al., U.S. Pat. No. 4,952,501.

Regulatory sequence: refers to a nucleic acid sequence that controls or regulates expression of structural genes when operably linked to those genes. These include, for example, the lac systems, the trp system, major operator and promoter regions of the phage lambda, the control region of fd coat protein and other sequences known to control the expression of genes in prokaryotic or eukaryotic cells. Regulatory sequences will vary depending on whether the vector is designed to express the operably linked gene in a prokaryotic or eukaryotic host, and may contain transcriptional elements such as enhancer elements, termination sequences, tissue-specificity elements and/or translational initiation and termination sites.

Sample: as used herein refers to a biological sample, such as, for example, tissue or fluid isolated from an individual or animal (including, without limitation, plasma, serum, cerebrospinal fluid, lymph, tears, nails, hair, saliva, milk, pus, and tissue exudates and secretions) or from in vitro cell culture-constituents, as well as samples obtained from, for example, a laboratory procedure.

Single nucleotide polymorphism (SNP): variation of a single nucleotide. This includes the replacement of one nucleotide by another and deletion or insertion of a single nucleotide. Typically, SNPs are biallelic markers although tri- and tetra-allelic markers also exist. For example, SNP A\C may comprise allele C or allele A (Tables 2, 3 and 7-10). Thus, a nucleic acid molecule comprising SNP A\C may include a C or A at the polymorphic position. For clarity purposes, an ambiguity code is used in Tables 2, 3 and 7-10 and the sequence listing, to represent the variations. For a combination of SNPs, the term “haplotype” is used, e.g. the genotype of the SNPs in a single DNA strand that are linked to one another. In certain embodiments, the term “haplotype” is used to describe a combination of SNP alleles, e.g., the alleles of the SNPs found together on a single DNA molecule. In specific embodiments, the SNPs in a haplotype are in linkage disequilibrium with one another.

Sequence-conservative: variants are those in which a change of one or more nucleotides in a given codon position results in no alteration in the amino acid encoded at that position (i.e., silent mutation).

Substantially homologous: a nucleic acid or fragment thereof is substantially homologous to another if, when optimally aligned (with appropriate nucleotide insertions and/or deletions) with the other nucleic acid (or its complementary strand), there is nucleotide sequence identity in at least 60% of the nucleotide bases, usually at least 70%, more usually at least 80%, preferably at least 90%, and more preferably at least 95-98% of the nucleotide bases. Alternatively, substantial homology exists when a nucleic acid or fragment thereof will hybridize, under selective hybridization conditions, to another nucleic acid (or a complementary strand thereof). Selectivity of hybridization exists when hybridization which is substantially more selective than total lack of specificity occurs. Typically, selective hybridization will occur when there is at least about 55% sequence identity over a stretch of at least about nine or more nucleotides, preferably at least about 65%, more preferably at least about 75%, and most preferably at least about 90% (M. Kanehisa, 1984, Nucl. Acids Res. 11:203-213). The length of homology comparison, as described, may be over longer stretches, and in certain embodiments will often be over a stretch of at least 14 nucleotides, usually at least 20 nucleotides, more usually at least 24 nucleotides, typically at least 28 nucleotides, more typically at least 32 nucleotides, and preferably at least 36 or more nucleotides.

Wild-type gene from Tables 4-6: refers to the reference sequence. The wild-type gene sequences from Tables 4-6 used to identify the variants (polymorphisms, alleles, and haplotypes) described in detail herein.

Technical and scientific terms used herein have the meanings commonly understood by one of ordinary skill in the art to which the present invention pertains, unless otherwise defined. Reference is made herein to various methodologies known to those of skill in the art. Publications and other materials setting forth such known methodologies to which reference is made are incorporated herein by reference in their entireties as though set forth in full, Standard reference works setting forth the general principles of recombinant DNA technology include J. Sambrook et al., 1989, Molecular Cloning: A Laboratory Manual, 2d Ed., Cold Spring Harbor Laboratory Press, Cold Spring Harbor, N.Y.; P. B. Kaufman et al., (eds), 1995, Handbook of Molecular and Cellular Methods in Biology and Medicine, CRC Press, Boca Raton; M. J. McPherson (ed), 1991, Directed Mutagenesis: A Practical Approach, IRL Press, Oxford; J. Jones, 1992, Amino Acid and Peptide Synthesis, Oxford Science Publications, Oxford; B. M. Austen and O. M. R. Westwood, 1991, Protein Targeting and Secretion, IRL Press, Oxford; D. N Glover (ed), 1985, DNA Cloning, Volumes I and 11; M. J. Gait (ed), 1984, Oligonucleotide Synthesis; B. D. Hames and S. J. Higgins (eds), 1984, Nucleic Acid Hybridization; Quirke and Taylor (eds), 1991, PCR-A Practical Approach; Harries and Higgins (eds), 1984, Transcription and Translation; R. I. Freshney (ed), 1986, Animal Cell Culture; Immobilized Cells and Enzymes, 1986, IRL Press; Perbal, 1984, A Practical Guide to Molecular Cloning, J. H. Miller and M. P. Calos (eds), 1987, Gene Transfer Vectors for Mammalian Cells, Cold Spring Harbor Laboratory Press; M. J. Bishop (ed), 1998, Guide to Human Genome Computing, 2d Ed., Academic Press, San Diego, Calif.; L. F. Peruski and A. H. Peruski, 1997, The Internet and the New Biology. Tools for Genomic and Molecular Research, American Society for Microbiology, Washington, D.C. Standard reference works setting forth the general principles of immunology include S. Sell, 1996, Immunology, Immunopathology & Immunity, 5th Ed., Appleton & Lange, Publ., Stamford, Conn.; D. Male et al., 1996, Advanced Immunology, 3d Ed., Times Mirror Intl Publishers Ltd., Publ., London; D. P. Stites and A. L Terr, 1991, Basic and Clinical Immunology, 7th Ed., Appleton & Lange, Publ., Norwalk, Conn.; and A. K. Abbas et al., 1991, Cellular and Molecular Immunology, W. B. Saunders Co., Publ., Philadelphia, Pa. Any suitable materials and/or methods known to those of skill can be utilized in carrying out the present invention; however, preferred materials and/or methods are described. Materials, reagents, and the like to which reference is made in the following description and examples are generally obtainable from commercial sources, and specific vendors are cited herein.

### DETAILED DESCRIPTION OF THE INVENTION

### General Description of Crohn's Disease

Inflammatory Bowel Disease (IBD) is characterized by excessive and chronic inflammation at various sites in the gastro-intestinal tract. IBD describes two clinical conditions called Crohn's disease (Crohn disease) and ulcerative colitis (UC). Crohn disease and UC share many clinical and pathological characteristics but they also have some markedly different features. There is strong scientific support suggesting that the main pathological processes in these two diseases are distinct. This patent application will focus primarily on Crohn's disease.

The United Kingdom, northern Europe, and North America have been reported to have the highest incidence rates and prevalence for Crohn disease. In North America, prevalence for Crohn disease ranges between 0.03% and 0.2%. However, reports of increasing incidence and prevalence from other areas of the world have been published over the past 30 years (reviewed in Loftus 2004). Crohn disease may occur in people of all ages, but it is most commonly diagnosed in late adolescence and early adulthood (reviewed in Andres and Friedman 1999). Any part of the gastrointestinal tract can be affected in Crohn disease, from the mouth to the anus, and patches of inflammation occur, interspersed with healthy tissue.

Most Crohn disease patients experience characteristic periods of remission and flare-ups of the disease, often-requiring long-term medication, and/or hospitalization and surgery. The symptoms and complications of Crohn's disease differ, depending on what part of the intestinal tract is inflamed. The severity of the disease does not correlate directly with the extent of bowel involvement. It is the disease pattern that is most important in determining the disease course and the nature of the associated complications. Thus, Crohn disease can be subdivided into 3 types: predominantly inflammatory Crohn disease, non-perforating Crohn disease (presence of strictures), or perforating Crohn disease (presence of fistulas and/or abscesses) (reviewed in Andres and Friedman 1999).

Crohn disease symptoms include chronic diarrhea, abdominal pain, cramping, anorexia, and weight loss. Systemic features include fatigue, tachycardia and pyrexia. Chronic or acute blood loss in the bowel may result in anemia and even shock. The most common complication of Crohn disease is the presence of strictures (obstruction) of the intestine due to swelling and the formation of scar tissue. Another complication involves sores or ulcers within the intestinal tract. Sometimes these deep ulcers turn into tracts called fistulas that connect different parts of the intestine. These fistulas often become infected and occasionally form an abscess. Extra-intestinal inflammatory manifestations can occur in joints, eyes, skin, mouth, and liver in patients with either forms of IBD (reviewed in Andres and Friedman 1999). Crohn disease patients also carry several risk factors for the development of osteoporosis such as calcium and vitamin D deficiency, and corticosteroid use (Tremaine 2003). Patients with Crohn disease are also at increased risk of cancer of both the small and the large intestine (reviewed in Andres and Friedman 1999). Crohn disease is associated with an increased mortality rate relative to the general population and independent of whether the small intestine, large intestine, or both are affected. The excess of mortality is most notable in the first few years after diagnosis and is most often attributable to complications of Crohn disease, including colorectal cancer as well as other gastrointestinal complications (reviewed in Andres and Friedman 1999). Crohn disease is a lifelong disease that causes symptoms that may interfere with social activities, interpersonal relationships, and employment. Impairment relates to disease severity, pattern and side-effects of medication, the possibility of surgery, but also to age, other demographic factors and co-morbid medical conditions, including depression and anxiety (Irvine 2004).

There is no single definitive test for the diagnosis of Crohn disease. To determine the diagnosis, physicians evaluate a combination of information from the history and physical examination of a patient and from results of endoscopic, radiologic and histologic (blood and tissue) tests. Endoscopy with biopsy is the cornerstone for diagnosing and evaluating disease activity in Crohn disease. Radiology tests are used together with endoscopy to help evaluate the small bowel and look at the entire abdomen for infections, strictures, obstructions, and fistulas. Because Crohn disease often mimics other conditions and symptoms may vary widely, it may take some time to confirm the diagnosis.

Because there is no cure for Crohn disease, the goal of medical treatment is to suppress the inflammatory response and alleviate the symptoms by decreasing the frequency of disease flare-ups and maintaining remissions. Non-surgical treatment for active disease involves the use of anti-inflammatory (aminosalicylates and corticosteroids), antimicrobial (antibiotics), and immunomodulatory agents to control symptoms and reduce disease activity. The biologic therapies are targeted towards specific disease mechanisms and have the potential to provide more effective and safe treatments for human diseases. Infliximab (Remicade®) is a chimeric monoclonal antibody against TNFalpha, and the first biologic therapy that was approved for Crohn disease. Several novel genetically engineered drugs targeting specific sites in the inflammatory cascade are likely to have an impact in the near future. Among them, anti-inflammatory cytokines (recombinant IL-10 and IL-11), antibodies (humanized IgG4, anti-TNFalpha, anti-alpha4-integrin) and antisense therapies (ICAM-1) are currently being evaluated in Crohn disease treatment (Sandborn and Faubion 2004).

The frequency of indications for surgery parallels the frequency of local intestinal complications of the disease. Surgery is never curative for Crohn disease because the disease frequently recurs at or near the site of surgery; its overall goal is to conserve bowel and return the individual to the best possible quality of life. Up to 74% of all patients eventually require surgical intervention for their disease (Farmer 1985), and nearly 30% of patients require surgery within the first year of diagnosis (Podolsky 1991).

Although the etiology of Crohn disease is poorly understood, studies indicate that Crohn disease pathogenesis is the result of the complex interaction between environmental factors (i.e. gut micro-flora), genetic susceptibility, and the immune system. It has been proposed that IBD results from a dys-regulated mucosal immune response to the intestinal micro-flora in genetically susceptible individuals. The inappropriate activation of the mucosal immune system observed in Crohn disease has been linked to a loss of tolerance to gut commensals. It also appears that the loss of mucosal integrity leading to translocation of bacteria in the bowel wall is a crucial step for the propagation of the inflammatory process. However, it is not known whether barrier function is first compromised by intrinsic defects in epithelial integrity, by infection with enteric pathogens, or by loss of commensal-dependent signals necessary to maintain the physical integrity of the epithelium and hypo-responsiveness of the mucosal immune system (reviewed in Bouma and Strober 2003).

Familial aggregation, twin studies and consistent ethnic differences in disease frequency have strongly supported the important role of genetic factors in the cause of Crohn disease (reviewed in Andres and Friedman 1999). However, the incomplete concordance for Crohn disease within monozygotic twins, the phenotypic variations and the observed familial pattern of non-Mendelian inheritance suggest that Crohn disease has a complex genetic basis with many contributing genes. These facts also underline the presence and importance of environmental factors in the pathogenesis of this disease, such as gut micro-flora as mentioned above, and cigarette smoking which is the best known environmental factor for Crohn disease (reviewed in Andres and Friedman 1999). In addition, disease heterogeneity in the phenotype (location, age of onset, number and types of surgery, behavior, extra-intestinal manifestations, response to class of medications) can reflect extensive genetic heterogeneity.

Many common diseases, like Crohn's disease, are complex genetic traits and are believed to involve several disease-genes rather than single genes, as is observed for rare diseases. This makes detection of any particular gene substantially more difficult than in a rare disease, where a single gene mutation that segregates according to a Mendelian inheritance pattern is the causative mutation. Any one of the multiple interacting gene mutations involved in the etiology of a complex disease will impart a lower relative risk for the disease than will the single gene mutation involved in a simple genetic disease. Low relative risk alleles are more difficult to detect and, as a result, the success of positional cloning using linkage mapping that was achieved for simple genetic disease genes has not been repeated for complex diseases.

Several approaches have been proposed to discover and characterize multiple genes in complex genetic traits. Genome-wide scans (GWS) have been shown to be efficient in identifying Crohn's disease susceptibility genes (NOD2/CARD15 and OCTN). Gene variants associated with Crohn disease have been reported for CARD15, SCL22A4/5 within the 5q31 haplotype, DLG5, MDR-1 and TNFSF15. The most consistent replication and the clearest functional data are available for the CARD15 gene. However, the genetic risk for Crohn disease has not yet been fully resolved. In view of its incomplete characterisation, more experiments are warranted to better understand the heritable basis of Crohn disease. Current technologies applied in the genetic epidemiology of complex disorders include systematic genome-wide linkage disequilibrium (LD)-based association scans and the analysis of coding SNPs (cSNPs) in candidate genes or gene regions, both of which have been successful employed, for instance, in the context of obesity and type I diabetes. Since even high-density LD-based mapping approaches are not capable of fully unraveling the genetic basis of a given disorder, genome-wide cSNP studies appear to be a meaningful accompaniment to the GWS approach, allowing a direct definition of susceptibility variants with a functional implication. In some respects, cSNP scans are more hypothesis-driven than LD-based approaches using non-coding SNPs so that the former may offer several advantages, for instance, in terms of a smaller number of statistical tests required to identify disease associations that are also easier to interpret.

The present invention reports the results of a genome-wide disease association analysis of 19,779 non-synonymous SNPs, and the GWS analysis on other populations, such as the Quebec Founder population (QFP) samples, that were performed in search for new susceptibility variants for Crohn disease.

A coding SNP in the ATG16L1 (‘autophagy 16-like’) gene was identified, which is significantly associated with an increased susceptibility for Crohn disease in different populations and which interacts statistically with variants in the known disease gene CARD15.

In view of the foregoing, identifying susceptibility genes and polymorphisms associated with Crohn's disease and their respective biochemical pathways will facilitate the identification of diagnostic markers as well as novel targets for improved therapeutics. It will also improve the quality of life for those afflicted by this disease and will reduce the economic costs of these afflictions at the individual and societal level. The identification of those genetic markers would provide the basis for novel genetic tests and eliminate or reduce the therapeutic methods currently used. The identification of those genetic markers will also provide the development of effective therapeutic intervention for the battery of laboratory, radiological, and endoscopic evaluations typically required to diagnose Crohn's disease. The present invention satisfies this need and provides related advantages as well.

Genome Wide Association Study to Identify Genes Associated with Crohn's Disease

The present invention is based on the discovery of genes associated with Crohn's disease. In the preferred embodiment, disease-associated loci (candidate region 1; Table 1) is identified by the statistically significant differences in allele or haplotype frequencies between the cases and the controls.

The invention also provides a method for the discovery of genes associated with Crohn's disease, comprising the following steps (see Example section herein):

Step 1: Recruit patients (cases) and controls
Step 2: DNA extraction and quantitation
Step 3: Genotype the recruited individuals
Step 4: Perform the genetic analysis on the results obtained
Step 5: Functional characterization of the associated genetic markers to identify causative polymorphisms

### The ATG16L1 Gene

A new susceptibility variant for Crohn disease (CD), in the ATG16L1 gene is identified in the present invention. Its disease association was also replicated in independent German, QFP and UK samples.

In addition, a statistical interaction between rs2241880 (the variant found associated in the ATG16L1 gene) and the established CARD15 disease mutations was noted (Table 10). Such interaction reflects a potential functional connection between the two encoded proteins at the molecular level.

In one embodiment, both rs2241880 and CARD15 mutations are associated with Crohn disease state and are functionally connected.

In another embodiment, rs2241880 alone is associated with Crohn disease state.

Both proteins are part of molecular pathways participating in the innate immune defence against intracellular bacteria. Bacteria that are able to invade the cytoplasm of host cells are recognized by the innate immune system. Proteins from then NLR (NACHT/LRR or NOD-like receptors) family recognize pathogen-associated molecular patterns (e.g. peptidoglycan) and lead to the activation of the innate immune defense. In particular, NOD2, the protein encoded by CARD15, plays a pivotal role in the detection of cytosolic Muramyl-Dipeptide (MurNAc-L-Ala-D-isoGln; MDP), a fragment of the bacterial cell wall. Autophagy is a fundamental molecular machinery of eukaryotes for bulk protein degradation. It has been implicated in diverse physiological processes such as organelle turnover, starvation response, cell death and defence against invading bacteria. Pathogens trapped by the autophagic membrane are ultimately targeted to the autolysosome compartment.

The ATG16L1 protein is part of this autophagosome pathway. Since variations in both CARD15 and ATG16L1 are not associated with ulcerative colitis, we hypothesize that genetic defects in the innate immune response against intracellular bacteria may be specific for Crohn disease.

The disease-associated variant rs2241880 leads to an amino acid exchange (Thr to Ala) at position 300 of the N-terminus of the WD-repeat domain in ATG16L1. The interaction partner of the WD domain in ATG16L1 has not yet been identified experimentally. It is clear, however, that the ATG12-ATG5 conjugate, which is required for autophagy, assembles in a multimeric complex with the coiled-coils protein ATG16. ATG16 interacts with the conjugate through ATG5, and ATG16 homooligomers formed by the coiled-coils connect multiple ATG12-ATG5 conjugates. Furthermore, it has been shown that the ATG12-ATG5ATG16 complex is necessary for autophagosome formation and localizes to the so-called preautophagosomal structure. In metazoans, yeast ATG16 is known as ATG16-like protein 1 (ATG16L1) because it contains an additional WD-repeat domain of as yet unidentified function at the C-terminus. In most WD-repeat proteins, seven or eight copies of the WD-repeat form a β-propeller domain structure with blades consisting of four-stranded anti-parallel β-sheets. Due to the circular arrangement of the propeller blades, the N-terminal strand β1 is included in the C-terminal blade, and this stable β-propeller structure provides an extensive surface for molecular interactions. These interactions may be impaired by the conformational change resulting from the T300A substitution.

Importantly, the association of a ATG16L1 variant with the disease and its interaction with CARD15 genotype support the emerging concept of Crohn disease as an inflammatory barrier disorder with a dysfunctional response to luminal bacterial content, and adds a new dimension because the autophagosome is now etiologically implicated.

The characterisation of rs2241880 as a disease variant for Crohn disease also supports the existing view of a strong link between autophagy and intracellular bacterial recognition molecules, such as CARD15. We therefore think that the findings presented here contribute to a better understanding of the aetiology of Crohn disease and, at the same time, stimulate the cell biological exploration of host-bacterial interaction.

### Nucleic Acid Sequences

The nucleic acid sequences of the present invention may be derived from a variety of sources including DNA, cDNA, synthetic DNA, synthetic RNA, derivatives, mimetics or combinations thereof. Such sequences may comprise genomic DNA, which may or may not include naturally occurring introns, genic regions, nongenic regions, and regulatory regions. Moreover, such genomic DNA may be obtained in association with promoter regions or poly (A) sequences. The sequences, genomic DNA, or cDNA may be obtained in any of several ways. Genomic DNA can be extracted and purified from suitable cells by means well known in the art. Alternatively, mRNA can be isolated from a cell and used to produce cDNA by reverse transcription or other means. The nucleic acids described herein are used in certain embodiments of the methods of the present invention for production of RNA, proteins or polypeptides, through incorporation into cells, tissues, or organisms. In one embodiment, DNA containing all or part of the coding sequence for the genes described in Tables 4-5, or the SNP markers described in Tables 2, 3 and 7-10, is incorporated into a vector for expression of the encoded polypeptide in suitable host cells. The invention also comprises the use of the nucleotide sequence of the nucleic acids of this invention to identify DNA probes for the genes described in Tables 4-6 or the SNP markers described in Tables 2, 3 and 7-10, PCR primers to amplify the genes described in Tables 4-6 or the SNP markers described in Tables 2, 3 and 7-10, nucleotide polymorphisms in the genes described in Tables 4-6, and regulatory elements of the genes described in Tables 4-6. The nucleic acids of the present invention find use as primers and templates for the recombinant production of Crohn's disease-associated peptides or polypeptides, for chromosome and gene mapping, to provide antisense sequences, for tissue distribution studies, to locate and obtain full length genes, to identify and obtain homologous sequences (wild-type and mutants), and in diagnostic applications.

### Antisense Oligonucleotides

In a particular embodiment of the invention, an antisense nucleic acid or oligonucleotide is wholly or partially complementary to, and can hybridize with, a target nucleic acid (either DNA or RNA) having the sequence of SEQ ID NO:1, NO:3 or any SEQ ID from any Tables of the invention. For example, an antisense nucleic acid or oligonucleotide comprising 16 nucleotides can be sufficient to inhibit expression of at least one gene from Tables 4-6. Alternatively, an antisense nucleic acid or oligonucleotide can be complementary to 5′ or 3′ untranslated regions, or can overlap the translation initiation codon (5′ untranslated and translated regions) of at least one gene from Tables 4-6, or its functional equivalent. In another embodiment, the antisense nucleic acid is wholly or partially complementary to, and can hybridize with, a target nucleic acid that encodes a polypeptide from a gene described in Tables 4-6.

In addition, oligonucleotides can be constructed which will bind to duplex nucleic acid (i.e., DNA:DNA or DNA:RNA), to form a stable triple helix containing or triplex nucleic acid. Such triplex oligonucleotides can inhibit transcription and/or expression of a gene from Tables 4-6, or its functional equivalent (M. D. Frank-Kamenetskii et al., 1995). Triplex oligonucleotides are constructed using the basepairing rules of triple helix formation and the nucleotide sequence of the genes described in Tables 4-6.

The present invention encompasses methods of using oligonucleotides in antisense inhibition of the function of the genes from Tables 4-6. In the context of this invention, the term “oligonucleotide” refers to naturally-occurring species or synthetic species formed from naturally-occurring subunits or their close homologs. The term may also refer to moieties that function similarly to oligonucleotides, but have non-naturally-occurring portions. Thus, oligonucleotides may have altered sugar moieties or inter-sugar linkages. Exemplary among these are phosphorothioate and other sulfur containing species which are known in the art. In preferred embodiments, at least one of the phosphodiester bonds of the oligonucleotide has been substituted with a structure that functions to enhance the ability of the compositions to penetrate into the region of cells where the RNA whose activity is to be modulated is located. It is preferred that such substitutions comprise phosphorothioate bonds, methyl phosphonate bonds, or short chain alkyl or cycloalkyl structures. In accordance with other preferred embodiments, the phosphodiester bonds are substituted with structures which are, at once, substantially non-ionic and non-chiral, or with structures which are chiral and enantiomerically specific. Persons of ordinary skill in the art will be able to select other linkages for use in the practice of the invention. Oligonucleotides may also include species that include at least some modified base forms. Thus, purines and pyrimidines other than those normally found in nature may be so employed. Similarly, modifications on the furanosyl portions of the nucleotide subunits may also be effected, as long as the essential tenets of this invention are adhered to. Examples of such modifications are 2′-O-alkyl- and 2′-halogen-substituted nucleotides. Some non-limiting examples of modifications at the 2′ position of sugar moieties which are useful in the present invention include OH, SH, SCH3, F, OCH3, OCN, O(CH2), NH2 and O(CH2)n CH3, where n is from 1 to about 10. Such oligonucleotides are functionally interchangeable with natural oligonucleotides or synthesized oligonucleotides, which have one or more differences from the natural structure. All such analogs are comprehended by this invention so long as they function effectively to hybridize with at least one gene from Tables 4-6 DNA or RNA to inhibit the function thereof.

The oligonucleotides in accordance with this invention preferably comprise from about 3 to about 50 subunits. It is more preferred that such oligonucleotides and analogs comprise from about 8 to about 25 subunits and still more preferred to have from about 12 to about 20 subunits. As defined herein, a “subunit” is a base and sugar combination suitably bound to adjacent subunits through phosphodiester or other bonds. Antisense nucleic acids or oligonucleotides can be produced by standard techniques (see, e.g., Shewmaker et al., U.S. Pat. No. 6,107,065). The oligonucleotides used in accordance with this invention may be conveniently and routinely made through the well-known technique of solid phase synthesis. Any other means for such synthesis may also be employed; however, the actual synthesis of the oligonucleotides is well within the abilities of the practitioner. It is also well known to prepare other oligonucleotides such as phosphorothioates and alkylated derivatives.

The oligonucleotides of this invention are designed to be hybridizable with RNA (e.g., mRNA) or DNA from genes described in Tables 4-6. For example, an oligonucleotide (e.g., DNA oligonucleotide) that hybridizes to mRNA from a gene described in Tables 4-6 can be used to target the mRNA for RnaseH digestion. Alternatively an oligonucleotide that can hybridize to the translation initiation site of the mRNA of a gene described in Tables 4-6 can be used to prevent translation of the mRNA. In another approach, oligonucleotides that bind to the double-stranded DNA of a gene from Tables 4-6 can be administered. Such oligonucleotides can form a triplex construct and inhibit the transcription of the DNA encoding polypeptides of the genes described in Tables 4-6. Triple helix pairing prevents the double helix from opening sufficiently to allow the binding of polymerases, transcription factors, or regulatory molecules. Recent therapeutic advances using triplex DNA have been described (see, e.g., J. E. Gee et al., 1994, Molecular and Immunologic Approaches, Futura Publishing Co., Mt. Kisco, N.Y.).

As non-limiting examples, antisense oligonucleotides may be targeted to hybridize to the following regions: mRNA cap region; translation initiation site; translational termination site; transcription initiation site; transcription termination site; polyadenylation signal; 3′ untranslated region; 5′ untranslated region; 5′ coding region; mid coding region; and 3′ coding region. Preferably, the complementary oligonucleotide is designed to hybridize to the most unique 5′ sequence of a gene described in Tables 4-6, including any of about 15-35 nucleotides spanning the 5′ coding sequence. In accordance with the present invention, the antisense oligonucleotide can be synthesized, formulated as a pharmaceutical composition, and administered to a subject. The synthesis and utilization of antisense and triplex oligonucleotides have been previously described (e.g., Simon et al., 1999; Barre et al., 2000; Elez et al., 2000; Sauter et al., 2000).

Alternatively, expression vectors derived from retroviruses, adenovirus, herpes or vaccinia viruses or from various bacterial plasmids may be used for delivery of nucleotide sequences to the targeted organ, tissue or cell population. Methods which are well known to those skilled in the art can be used to construct recombinant vectors which will express nucleic acid sequence that is complementary to the nucleic acid sequence encoding a polypeptide from the genes described in Tables 4-6. These techniques are described both in Sambrook et al., 1989 and in Ausubel et al., 1992. For example, expression of at least one gene from Tables 4-6 can be inhibited by transforming a cell or tissue with an expression vector that expresses high levels of untranslatable sense or antisense sequences. Even in the absence of integration into the DNA, such vectors may continue to transcribe RNA molecules until they are disabled by endogenous nucleases. Transient expression may last for a month or more with a nonreplicating vector, and even longer if appropriate replication elements are included in the vector system. Various assays may be used to test the ability of gene-specific antisense oligonucleotides to inhibit the expression of at least one gene from Tables 4-6. For example, mRNA levels of the genes described in Tables 4-6 can be assessed by Northern blot analysis (Sambrook et al., 1989; Ausubel et al., 1992; J. C. Alwine et al. 1977; I. M. Bird, 1998), quantitative or semi-quantitative RT-PCR analysis (see, e.g., W. M. Freeman et al., 1999; Ren et al., 1998; J. M. Cale et al., 1998), or in situ hybridization (reviewed by A. K. Raap, 1998). Alternatively, antisense oligonucleotides may be assessed by measuring levels of the polypeptide from the genes described in Tables 4-6, e.g., by western blot analysis, indirect immunofluorescence and immunoprecipitation techniques (see, e.g., J. M. Walker, 1998, Protein Protocols on Crohn disease-ROM, Humana Press, Totowa, N.J.). Any other means for such detection may also be employed, and is well within the abilities of the practitioner.

Methods to Identify Agents that Modulate the Expression of a Nucleic Acid Encoding a Gene Involved in Crohn's Disease.

The present invention provides methods for identifying agents that modulate the expression of a nucleic acid encoding a gene from Tables 4-6. Such methods may utilize any available means of monitoring for changes in the expression level of the nucleic acids of the invention. As used herein, an agent is said to modulate the expression of a nucleic acid of the invention if it is capable of up- or down-regulating expression of the nucleic acid in a cell. Such cells can be obtained from any parts of the body such as the GI track, colon, esophagus, stomach, rectum, jujenum, ileum, mucosa, submucosa, cecum, rectum, scalp, blood, dermis, epidermis, skin cells, cutaneous surfaces, intertrigious areas, genitalia, vessels and endothelium. Some non-limiting examples of cells that can be used are: muscle cells, nervous cells, blood and vessels cells, dermis, epidermis and other skin cells, T cell, mast cell, Crohn disease4+ lymphocyte, monocyte, macrophage, synovial cell, glial cell, villous intestinal cell, neutrophilic granulocyte, eosinophilic granulocyte, keratinocyte, lamina propria lymphocyte, intraepithelial lymphocyte, epithelial cells and lymphocytes.

In one assay format, the expression of a nucleic acid encoding a gene of the invention (see Tables 4-6) in a cell or tissue sample is monitored directly by hybridization to the nucleic acids of the invention. Cell lines or tissues are exposed to the agent to be tested under appropriate conditions and time and total RNA or mRNA is isolated by standard procedures such as those disclosed in Sambrook et al., (1989) Molecular Cloning: A Laboratory Manual, Cold Spring Harbor Laboratory Press).

Probes to detect differences in RNA expression levels between cells exposed to the agent and control cells may be prepared as described above. Hybridization conditions are modified using known methods, such as those described by Sambrook et al., and Ausubel et al., as required for each probe. Hybridization of total cellular RNA or RNA enriched for polyA RNA can be accomplished in any available format. For instance, total cellular RNA or RNA enriched for polyA RNA can be affixed to a solid support and the solid support exposed to at least one probe comprising at least one, or part of one of the sequences of the invention under conditions in which the probe will specifically hybridize. Alternatively, nucleic acid fragments comprising at least one, or part of one of the sequences of the invention can be affixed to a solid support, such as a silicon chip or a porous glass wafer. The chip or wafer can then be exposed to total cellular RNA or polyA RNA from a sample under conditions in which the affixed sequences will specifically hybridize to the RNA. By examining for the ability of a given probe to specifically hybridize to an RNA sample from an untreated cell population and from a cell population exposed to the agent, agents which up or down regulate expression are identified.

Methods to Identify Agents that Modulate the Activity of a Protein Encoded by a Gene Involved in Crohn's Disease.

The present invention provides methods for identifying agents that modulate at least one activity of the proteins described in Tables 4-6. Such methods may utilize any means of monitoring or detecting the desired activity. As used herein, an agent is said to modulate the expression of a protein of the invention if it is capable of up- or down-regulating expression of the protein in a cell. Such cells can be obtained from any parts of the body such as the GI track, colon, esophagus, stomach, rectum, jujenum, ileum, mucosa, submucosa, cecum, rectum, scalp, blood, dermis, epidermis, skin cells, cutaneous surfaces, intertrigious areas, genitalia, vessels and endothelium. Some non-limiting examples of cells that can be used are: muscle cells, nervous cells, blood and vessels cells, dermis, epidermis and other skin cells, T cell, mast cell, Crohn disease4+ lymphocyte, monocyte, macrophage, synovial cell, glial cell, villous intestinal cell, neutrophilic granulocyte, eosinophilic granulocyte, keratinocyte, lamina propria lymphocyte, intraepithelial lymphocyte, epithelial cells and lymphocytes.

In one format, the specific activity of a protein of the invention, normalized to a standard unit, may be assayed in a cell population that has been exposed to the agent to be tested and compared to an unexposed control cell population may be assayed. Cell lines or populations are exposed to the agent to be tested under appropriate conditions and times. Cellular lysates may be prepared from the exposed cell line or population and a control, unexposed cell line or population. The cellular lysates are then analyzed with the probe.

Antibody probes can be prepared by immunizing suitable mammalian hosts utilizing appropriate immunization protocols using the proteins of the invention or antigen-containing fragments thereof. To enhance immunogenicity, these proteins or fragments can be conjugated to suitable carriers. Methods for preparing immunogenic conjugates with carriers such as BSA, KLH or other carrier proteins are well known in the art. In some circumstances, direct conjugation using, for example, carbodiimide reagents may be effective; in other instances linking reagents such as those supplied by Pierce Chemical Co. (Rockford, Ill.) may be desirable to provide accessibility to the hapten. The hapten peptides can be extended at either the amino or carboxy terminus with a cysteine residue or interspersed with cysteine residues, for example, to facilitate linking to a carrier. Administration of the immunogens is conducted generally by injection over a suitable time period and with use of suitable adjuvants, as is generally understood in the art. During the immunization schedule, titers of antibodies are taken to determine adequacy of antibody formation. While the polyclonal antisera produced in this way may be satisfactory for some applications, for pharmaceutical compositions, use of monoclonal preparations is preferred. Immortalized cell lines which secrete the desired monoclonal antibodies may be prepared using standard methods, see e.g., Kohler & Milstein (1992) or modifications which affect immortalization of lymphocytes or spleen cells, as is generally known. The immortalized cell lines secreting the desired antibodies can be screened by immunoassay in which the antigen is the peptide hapten, polypeptide or protein. When the appropriate immortalized cell culture secreting the desired antibody is identified, the cells can be cultured either in vitro or by production in ascites fluid. The desired monoclonal antibodies may be recovered from the culture supernatant or from the ascites supernatant. Fragments of the monoclonal antibodies or the polyclonal antisera which contain the immunologically significant portion(s) can be used as antagonists, as well as the intact antibodies. Use of immunologically reactive fragments, such as Fab or Fab′ fragments, is often preferable, especially in a therapeutic context, as these fragments are generally less immunogenic than the whole immunoglobulin. The antibodies or fragments may also be produced, using current technology, by recombinant means. Antibody regions that bind specifically to the desired regions of the protein can also be produced in the context of chimeras derived from multiple species. Antibody regions that bind specifically to the desired regions of the protein can also be produced in the context of chimeras from multiple species, for instance, humanized antibodies. The antibody can therefore be a humanized antibody or a human antibody, as described in U.S. Pat. No. 5,585,089 or Riechmann et al. (1988).

Agents that are assayed in the above method can be randomly selected or rationally selected or designed. As used herein, an agent is said to be randomly selected when the agent is chosen randomly without considering the specific sequences involved in the association of the protein of the invention alone or with its associated substrates, binding partners, etc. An example of randomly selected agents is the use of a chemical library or a peptide combinatorial library, or a growth broth of an organism. As used herein, an agent is said to be rationally selected or designed when the agent is chosen on a non-random basis which takes into account the sequence of the target site or its conformation in connection with the agent's action. Agents can be rationally selected or rationally designed by utilizing the peptide sequences that make up these sites. For example, a rationally selected peptide agent can be a peptide whose amino acid sequence is identical to or a derivative of any functional consensus site. The agents of the present invention can be, as examples, oligonucleotides, antisense polynucleotides, interfering RNA, peptides, peptide mimetics, antibodies, antibody fragments, small molecules, vitamin derivatives, as well as carbohydrates. Peptide agents of the invention can be prepared using standard solid phase (or solution phase) peptide synthesis methods, as is known in the art. In addition, the DNA encoding these peptides may be synthesized using commercially available oligonucleotide synthesis instrumentation and produced recombinantly using standard recombinant production systems. The production using solid phase peptide synthesis is necessitated if non-gene-encoded amino acids are to be included.

Another class of agents of the present invention includes antibodies or fragments thereof that bind to a protein encoded by a gene in Tables 4-6. Antibody agents can be obtained by immunization of suitable mammalian subjects with peptides, containing as antigenic regions, those portions of the protein intended to be targeted by the antibodies (see section above of antibodies as probes for standard antibody preparation methodologies).

In yet another class of agents, the present invention includes peptide mimetics that mimic the three-dimensional structure of the protein encoded by a gene from Tables 4-6. Such peptide mimetics may have significant advantages over naturally occurring peptides, including, for example: more economical production, greater chemical stability, enhanced pharmacological properties (half-life, absorption, potency, efficacy, etc.), altered specificity (e.g., a broad-spectrum of biological activities), reduced antigenicity and others. In one form, mimetics are peptide-containing molecules that mimic elements of protein secondary structure. The underlying rationale behind the use of peptide mimetics is that the peptide backbone of proteins exists chiefly to orient amino acid side chains in such a way as to facilitate molecular interactions, such as those of antibody and antigen. A peptide mimetic is expected to permit molecular interactions similar to the natural molecule. In another form, peptide analogs are commonly used in the pharmaceutical industry as non-peptide drugs with properties analogous to those of the template peptide. These types of non-peptide compounds are also referred to as peptide mimetics or peptidomimetics (Fauchere, 1986; Veber & Freidinger, 1985; Evans et al., 1987) which are usually developed with the aid of computerized molecular modeling. Peptide mimetics that are structurally similar to therapeutically useful peptides may be used to produce an equivalent therapeutic or prophylactic effect. Generally, peptide mimetics are structurally similar to a paradigm polypeptide (i.e., a polypeptide that has a biochemical property or pharmacological activity), but have one or more peptide linkages optionally replaced by a linkage using methods known in the art. Labeling of peptide mimetics usually involves covalent attachment of one or more labels, directly or through a spacer (e.g., an amide group), to non-interfering position(s) on the peptide mimetic that are predicted by quantitative structure-activity data and molecular modeling. Such non-interfering positions generally are positions that do not form direct contacts with the macromolecule(s) to which the peptide mimetic binds to produce the therapeutic effect. Derivitization (e.g., labeling) of peptide mimetics should not substantially interfere with the desired biological or pharmacological activity of the peptide mimetic. The use of peptide mimetics can be enhanced through the use of combinatorial chemistry to create drug libraries. The design of peptide mimetics can be aided by identifying amino acid mutations that increase or decrease binding of the protein to its binding partners. Approaches that can be used include the yeast two hybrid method (see Chien et al., 1991) and the phage display method. The two hybrid method detects protein-protein interactions in yeast (Fields et al., 1989). The phage display method detects the interaction between an immobilized protein and a protein that is expressed on the surface of phages such as lambda and M13 (Amberg et al., 1993; Hogrefe et al., 1993). These methods allow positive and negative selection for protein-protein interactions and the identification of the sequences that determine these interactions.

### Method to Diagnose Crohn's Disease

The present invention also relates to methods for diagnosing inflammatory bowel disease or a related disease, preferably Crohn's disease (Crohn disease), a disposition to such disease, predisposition to such a disease and/or disease progression. In some methods, the steps comprise contacting a target sample with (a) nucleic acid molecule(s) or fragments thereof and comparing the concentration of individual mRNA(s) with the concentration of the corresponding mRNA(s) from at least one healthy donor. An aberrant (increased or decreased) mRNA level of at least one gene from Tables 4-6, or at least 5 or 10 genes from Tables 4-6, determined in the sample in comparison to the control sample is an indication of Crohn's disease or a related disease or a disposition to such kinds of diseases. For diagnosis, samples are, preferably, obtained from inflamed colon tissue. Samples can also be obtained from any parts of the body such as the GI track, colon, esophagus, stomach, rectum, jujenum, ileum, mucosa, submucosa, cecum, rectum, scalp, blood, dermis, epidermis, skin cells, cutaneous surfaces, intertrigious areas, genitalia, vessels and endothelium. Some non-limiting examples of cells that can be used are: muscle cells, nervous cells, blood and vessels cells, dermis, epidermis and other skin cells, T cell, mast cell, Crohn disease4+ lymphocyte, monocyte, macrophage, synovial cell, glial cell, villous intestinal cell, neutrophilic granulocyte, eosinophilic granulocyte, keratinocyte, lamina propria lymphocyte, intraepithelial lymphocyte, epithelial cells and lymphocytes.

For analysis of gene expression, total RNA is obtained from cells according to standard procedures and, preferably, reverse-transcribed. Preferably, a DNAse treatment (in order to get rid of contaminating genomic DNA) is performed. Some non-limiting examples of cells that can be used are: muscle cells, nervous cells, blood and vessels cells, dermis, epidermis and other skin cells, T cell, mast cell, Crohn disease4+ lymphocyte, monocyte, macrophage, synovial cell, glial cell, villous intestinal cell, neutrophilic granulocyte, eosinophilic granulocyte, keratinocyte, lamina propria lymphocyte, intraepithelial lymphocyte, epithelial cells and lymphocytes.

The nucleic acid molecule or fragment is typically a nucleic acid probe for hybridization or a primer for PCR. The person skilled in the art is in a position to design suitable nucleic acids probes based on the information provided in the Tables of the present invention. The target cellular component, i.e. mRNA, e.g., in colon tissue, may be detected directly in situ, e.g. by in situ hybridization or it may be isolated from other cell components by common methods known to those skilled in the art before contacting with a probe. Detection methods include Northern blot analysis, RNase protection, in situ methods, e.g. in situ hybridization, in vitro amplification methods (PCR, LCR, QRNA replicase or RNA-transcription/amplification (TAS, 3SR), reverse dot blot disclosed in EP-B10237362) and other detection assays that are known to those skilled in the art. Products obtained by in vitro amplification can be detected according to established methods, e.g. by separating the products on agarose or polyacrylamide gels and by subsequent staining with ethidium bromide. Alternatively, the amplified products can be detected by using labeled primers for amplification or labeled dNTPs. Preferably, detection is based on a microarray.

The probes (or primers) (or, alternatively, the reverse-transcribed sample mRNAs) can be detectably labeled, for example, with a radioisotope, a bioluminescent compound, a chemiluminescent compound, a fluorescent compound, a metal chelate, or an enzyme.

The present invention also relates to the use of the nucleic acid molecules or fragments described above for the preparation of a diagnostic composition for the diagnosis of Crohn's disease or a disposition to such a disease.

The present invention also relates to the use of the nucleic acid molecules of the present invention for the isolation or development of a compound which is useful for therapy of Crohn's disease. For example, the nucleic acid molecules of the invention and the data obtained using said nucleic acid molecules for diagnosis of Crohn's disease might allow for the identification of further genes which are specifically dysregulated, and thus may be considered as potential targets for therapeutic interventions.

The invention further provides prognostic assays that can be used to identify subjects having or at risk of developing Crohn's disease. In such method, a test sample is obtained from a subject and the amount and/or concentration of the nucleic acid described in Tables 4-6 is determined; wherein the presence of an associated allele, a particular allele of a polymorphic locus, or the likes in the nucleic acids sequences of this invention can be diagnostic for a subject having or at risk of developing Crohn's. As used herein, a “test sample” refers to a biological sample obtained from a subject of interest. For example, a test sample can be a biological fluid, a cell sample, or tissue. A biological fluid can be, but is not limited to saliva, serum, mucus, urine, stools, spermatozoids, vaginal secretions, lymph, amiotic liquid, pleural liquid and tears. Cells can be, but are not limited to: muscle cells, nervous cells, blood and vessels cells, dermis, epidermis and other skin cells, T cell, mast cell, Crohn disease4+ lymphocyte, monocyte, macrophage, synovial cell, glial cell, villous intestinal cell, neutrophilic granulocyte, eosinophilic granulocyte, keranocyte, lamina propria lymphocyte, intraephitelial lymphocyte, epithelial cells and lymphocytes.

Furthermore, the prognostic assays described herein can be used to determine whether a subject can be administered an agent (e.g., an agonist, antagonist, peptidomimetic, polypeptide, nucleic acid such as antisense DNA or interfering RNA (RNAi), small molecule or other drug candidate) to treat Crohn's disease. Specifically, these assays can be used to predict whether an individual will have an efficacious response or will experience adverse events in response to such an agent. For example, such methods can be used to determine whether a subject can be effectively treated with an agent that modulates the expression and/or activity of a gene from Tables 4-6 or the nucleic acids described herein. In another example, an association study may be performed to identify polymorphisms from Tables 2, 3 and 7-10 that are associated with a given response to the agent, e.g., an efficacious response or the likelihood of one or more adverse events. Thus, one embodiment of the present invention provides methods for determining whether a subject can be effectively treated with an agent for a disorder associated with aberrant expression or activity of a gene from Tables 4-6 in which a test sample is obtained and nucleic acids or polypeptides from Tables 4-6 are detected (e.g., wherein the presence of a particular level of expression of a gene from Tables 4-6 or a particular allelic variant of such gene, such as polymorphisms from Tables 2, 3 and 7-10 is diagnostic for a subject that can be administered an agent to treat a disorder such as Crohn's disease). In one embodiment, the method includes obtaining a sample from a subject suspected of having Crohn's disease or an affected individual and exposing such sample to an agent. The expression and/or activity of the nucleic acids and/or genes of the invention are monitored before and after treatment with such agent to assess the effect of such agent. After analysis of the expression values, one skilled in the art can determine whether such agent can effectively treat such subject. In another embodiment, the method includes obtaining a sample from a subject having or susceptible to developing Crohn's disease and determining the allelic constitution of polymorphisms from Tables 2, 3 and 7-10 that are associated with a particular response to an agent. After analysis of the allelic constitution of the individual at the associated polymorphisms, one skilled in the art can determine whether such agent can effectively treat such subject.

The methods of the invention can also be used to detect genetic alterations in a gene from Tables 4-6, thereby determining if a subject with the lesioned gene is at risk for a disorder associated with Crohn's disease. In preferred embodiments, the methods include detecting, in a sample of cells from the subject, the presence or absence of a genetic alteration characterized by at least one alteration linked to or affecting the integrity of a gene from Tables 4-6 encoding a polypeptide or the misexpression of such gene. For example, such genetic alterations can be detected by ascertaining the existence of at least one of: (1) a deletion of one or more nucleotides from a gene from Tables 4-6; (2) an addition of one or more nucleotides to a gene from Tables 4-6; (3) a substitution of one or more nucleotides of a gene from Tables 4-6; (4) a chromosomal rearrangement of a gene from Tables 4-6; (5) an alteration in the level of a messenger RNA transcript of a gene from Tables 4-6; (6) aberrant modification of a gene from Tables 4-6, such as of the methylation pattern of the genomic DNA, (7) the presence of a non-wild type splicing pattern of a messenger RNA transcript of a gene from Tables 4-6; (8) inappropriate post-translational modification of a polypeptide encoded by a gene from Tables 4-6; and (9) alternative promoter use. As described herein, there are a large number of assay techniques known in the art which can be used for detecting alterations in a gene from Tables 4-6. A preferred biological sample is a peripheral blood sample obtained by conventional means from a subject. Another preferred biological sample is a buccal swab. Other biological samples can be, but are not limited to, urine, stools, spermatozoids, vaginal secretions, lymph, amiotic liquid, pleural liquid and tears.

In certain embodiments, detection of the alteration involves the use of a probe/primer in a polymerase chain reaction (PCR) (see, e.g., U.S. Pat. Nos. 4,683,195 and 4,683,202), such as anchor PCR or RACE PCR, or alternatively, in a ligation chain reaction (LCR) (see, e.g., Landegran et al., 1988; and Nakazawa et al., 1994), the latter of which can be particularly useful for detecting point mutations in a gene from Tables 4-6 (see Abavaya et al., 1995). This method can include the steps of collecting a sample of cells from a patient, isolating nucleic acid (e.g., genomic DNA, mRNA, or both) from the cells of the sample, contacting the nucleic acid sample with one or more primers which specifically hybridize to a gene from Tables 4-6 under conditions such that hybridization and amplification of the nucleic acid from Tables 4-6 (if present) occurs, and detecting the presence or absence of an amplification product, or detecting the size of the amplification product and comparing the length to a control sample. PCR and/or LCR may be desirable to use as a preliminary amplification step in conjunction with some of the techniques used for detecting a mutation, an associated allele, a particular allele of a polymorphic locus, or the like described herein.

Alternative amplification methods include: self sustained sequence replication (Guatelli et al., 1990), transcriptional amplification system (Kwoh et al., 1989), Q-Beta Replicase (Lizardi et al., 1988), isothermal amplification (e.g. Dean et al., 2002); and Hafner et al., 2001), or any other nucleic acid amplification method, followed by the detection of the amplified molecules using techniques well known to those of ordinary skill in the art. These detection schemes are especially useful for the detection of nucleic acid molecules if such molecules are present in very low number.

In an alternative embodiment, alterations in a gene from Tables 4-6, from a sample cell can be identified by identifying changes in a restriction enzyme cleavage pattern. For example, sample and control DNA is isolated, amplified (optionally), digested with one or more restriction endonucleases, and fragment length sizes are determined by gel electrophoresis and compared. Differences in fragment length sizes between sample and control DNA indicate a mutation(s), an associated allele, a particular allele of a polymorphic locus, or the like in the sample DNA. Moreover, sequence specific ribozymes (see, e.g., U.S. Pat. No. 5,498,531 or DNAzyme e.g. U.S. Pat. No. 5,807,718) can be used to score for the presence of specific associated allele, a particular allele of a polymorphic locus, or the likes by development or loss of a ribozyme or DNAzyme cleavage site.

The present invention also relates to further methods for diagnosing Crohn's disease, a disposition to such disorder, predisposition to such a disorder and/or disorder progression. In some methods, the steps comprise contacting a target sample with (a) nucleic molecule(s) or fragments thereof and determining the presence or absence of a particular allele of a polymorphism that confers a disorder-related phenotype (e.g., predisposition to such a disorder and/or disorder progression). The presence of at least one allele from Tables 2, 3 and 7-10 that is associated with Crohn's disease (“associated allele”), at least 5 or 10 associated alleles from Tables 2, 3 and 7-10, at least 50 associated alleles from Tables 2, 3 and 7-10 at least 100 associated alleles from Table Tables 2, 3 and 7-10, or at least 200 associated alleles from Table Tables 2, 3 and 7-10 determined in the sample is an indication of Crohn's disease, a disposition or predisposition to such kinds of disorders, or a prognosis for such disorder progression. Samples may be obtained from any parts of the body such as the GI track, colon, esophagus, stomach, rectum, jujenum, ileum, mucosa, submucosa, cecum, rectum, scalp, blood, dermis, epidermis, skin cells, cutaneous surfaces, intertrigious areas, genitalia, vessels and endothelium. Some non-limiting examples of cells that can be used are: muscle cells, nervous cells, blood and vessels cells, dermis, epidermis and other skin cells, T cell, mast cell, Crohn disease4+ lymphocyte, monocyte, macrophage, synovial cell, glial cell, villous intestinal cell, neutrophilic granulocyte, eosinophilic granulocyte, keratinocyte, lamina propria lymphocyte, intraepithelial lymphocyte, epithelial cells and lymphocytes.

In other embodiments, alterations in a gene from Tables 4-6 can be identified by hybridizing sample and control nucleic acids, e.g., DNA or RNA, to high density arrays or bead arrays containing tens to thousands of oligonucleotide probes (Cronin et al., 1996; Kozal et al., 1996). For example, alterations in a gene from Tables 4-6 can be identified in two dimensional arrays containing light-generated DNA probes as described in Cronin et al., (1996). Briefly, a first hybridization array of probes can be used to scan through long stretches of DNA in a sample and control to identify base changes between the sequences by making linear arrays of sequential overlapping probes. This step allows the identification of point mutations, associated alleles, particular alleles of a polymorphic locus, or the like. This step is followed by a second hybridization array that allows the characterization of specific mutations by using smaller, specialized probe arrays complementary to all variants, mutations, alleles detected. Each mutation array is composed of parallel probe sets, one complementary to the wild-type gene and the other complementary to the mutant gene.

In yet another embodiment, any of a variety of sequencing reactions known in the art can be used to directly sequence a gene from Tables 4-6 and detect an associated allele, a particular allele of a polymorphic locus, or the like by comparing the sequence of the sample gene from Tables 4-6 with the corresponding wild-type (control) sequence. Examples of sequencing reactions include those based on techniques developed by Maxam and Gilbert (1977) or Sanger (1977). It is also contemplated that any of a variety of automated sequencing procedures can be utilized when performing the diagnostic assays (Bio/Techniques 19:448, 1995) including sequencing by mass spectrometry (see, e.g. PCT International Publication No. WO 94/16101; Cohen et al., 1996; and Griffin et al. 1993), real-time pyrophosphate sequencing method (Ronaghi et al., 1998; and Permutt et al., 2001) and sequencing by hybridization (see e.g. Drmanac et al., 2002).

Other methods of detecting an associated allele, a particular allele of a polymorphic locus, or the likes in a gene from Tables 4-6 include methods in which protection from cleavage agents is used to detect mismatched bases in RNA/RNA, DNA/DNA or RNA/DNA heteroduplexes (Myers et al., 1985). In general, the technique of “mismatch cleavage” starts by providing heteroduplexes formed by hybridizing (labeled) RNA or DNA containing the wild-type gene sequence from Tables 4-6 with potentially mutant RNA or DNA obtained from a tissue sample. The double-stranded duplexes are treated with an agent that cleaves single-stranded regions of the duplex such as which will exist due to basepair mismatches between the control and sample strands. For instance, RNA/DNA duplexes can be treated with RNase and DNA/DNA hybrids treated with S1 nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of an associated allele, a particular allele of a polymorphic locus, or the like (see, for example, Cotton et al., 1988; Saleeba et al., 1992). In a preferred embodiment, the control DNA or RNA can be labeled for detection, as described herein.

In still another embodiment, the mismatch cleavage reaction employs one or more proteins that recognize mismatched base pairs in double-stranded DNA (so called “DNA mismatch repair” enzymes) in defined systems for detecting and mapping point an associated allele, a particular allele of a polymorphic locus, or the likes in a gene from Tables 4-6 cDNAs obtained from samples of cells. For example, the mutY enzyme of E. coli cleaves A at G/A mismatches (Hsu et al., 1994). Other examples include, but are not limited to, the MutHLS enzyme complex of E. coli (Smith and Modrich., 1996) and Cel 1 from the celery (Kulinski et al., 2000) both cleave the DNA at various mismatches. According to an exemplary embodiment, a probe based on a gene sequence from Tables 4-6 is hybridized to a cDNA or other DNA product from a test cell or cells. The duplex is treated with a DNA mismatch repair enzyme, and the cleavage products, if any, can be detected using electrophoresis protocols or the like. See, for example, U.S. Pat. No. 5,459,039. Alternatively, the screen can be performed in vivo following the insertion of the heteroduplexes in an appropriate vector. The whole procedure is known to those ordinary skilled in the art and is referred to as mismatch repair detection (see e.g. Fakhrai-Rad et al., 2004).

In other embodiments, alterations in electrophoretic mobility can be used to identify an associated allele, a particular allele of a polymorphic locus, or the likes in genes from Tables 4-6. For example, single strand conformation polymorphism (SSCP) analysis can be used to detect differences in electrophoretic mobility between mutant and wild type nucleic acids (Orita et al., 1993; see also Cotton, 1993; and Hayashi et al., 1992). Single-stranded DNA fragments of sample and control nucleic acids from Tables 4-6 will be denatured and allowed to renature. The secondary structure of single-stranded nucleic acids varies according to sequence; the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the method utilizes heteroduplex analysis to separate double stranded heteroduplex molecules on the basis of changes in electrophoretic mobility (Kee et al., 1991).

In yet another embodiment, the movement of mutant or wild-type fragments in a polyacrylamide gel containing a gradient of denaturant is assayed using denaturing gradient gel electrophoresis (DGGE) (Myers et al., 1985). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum et al., 1987). In another embodiment, the mutant fragment is detected using denaturing HPLC (see e.g. Hoogendoorn et al., 2000).

Examples of other techniques for detecting point mutations, an associated allele, a particular allele of a polymorphic locus, or the like include, but are not limited to, selective oligonucleotide hybridization, selective amplification, selective primer extension, selective ligation, single-base extension, selective termination of extension or invasive cleavage assay. For example, oligonucleotide primers may be prepared in which the known associated allele, particular allele of a polymorphic locus, or the like is placed centrally and then hybridized to target DNA under conditions which permit hybridization only if a perfect match is found (Saiki et al., 1986; Saiki et al., 1989). Such allele specific oligonucleotides are hybridized to PCR amplified target DNA of a number of different associated alleles, a particular allele of a polymorphic locus, or the likes where the oligonucleotides are attached to the hybridizing membrane and hybridized with labeled target DNA. Alternatively, the amplification, the allele-specific hybridization and the detection can be done in a single assay following the principle of the 5′ nuclease assay (e.g. see Livak et al., 1995). For example, the associated allele, a particular allele of a polymorphic locus, or the like locus is amplified by PCR in the presence of both allele-specific oligonucleotides, each specific for one or the other allele. Each probe has a different fluorescent dye at the 5′ end and a quencher at the 3′ end. During PCR, if one or the other or both allele-specific oligonucleotides are hybridized to the template, the Taq polymerase via its 5′ exonuclease activity will release the corresponding dyes. The latter will thus reveal the genotype of the amplified product.

The hybridization may also be carried out with a temperature gradient following the principle of dynamic allele-specific hybridization or like (e.g. Jobs et al., 2003; and Bourgeois and Labuda, 2004). For example, the hybridization is done using one of the two allele-specific oligonucleotides labeled with a fluorescent dye, an intercalating quencher under a gradually increasing temperature. At low temperature, the probe is hybridized to both the mismatched and full-matched template. The probe melts at a lower temperature when hybridized to the template with a mismatch. The release of the probe is captured by an emission of the fluorescent dye, away from the quencher. The probe melts at a higher temperature when hybridized to the template with no mismatch. The temperature-dependent fluorescence signals therefore indicate the absence or presence of the associated allele, particular allele of a polymorphic locus, or the like (e.g. Jobs et al. supra). Alternatively, the hybridization is done under a gradually decreasing temperature. In this case, both allele-specific oligonucleotides are hybridized to the template competitively. At high temperature none of the two probes is hybridized. Once the optimal temperature of the full-matched probe is reached, it hybridizes and leaves no target for the mismatched probe. In the latter case, if the allele-specific probes are differently labeled, then they are hybridized to a single PCR-amplified target. If the probes are labeled with the same dye, then the probe cocktail is hybridized twice to identical templates with only one labeled probe, different in the two cocktails, in the presence of the unlabeled competitive probe.

Alternatively, allele specific amplification technology that depends on selective PCR amplification may be used in conjunction with the present invention, Oligonucleotides used as primers for specific amplification may carry the associated allele, particular allele of a polymorphic locus, or the like of interest in the center of the molecule, so that amplification depends on differential hybridization (Gibbs et al., 1989) or at the extreme 3′ end of one primer where, under appropriate conditions, mismatch can prevent, or reduce polymerase extension (Prossner, 1993). In addition it may be desirable to introduce a novel restriction site in the region of the associated allele, particular allele of a polymorphic locus, or the like to create cleavage-based detection (Gasparini et al., 1992). It is anticipated that in certain embodiments amplification may also be performed using Taq ligase for amplification (Barany, 1991). In such cases, ligation will occur only if there is a perfect match at the 3′ end of the 5′ sequence making it possible to detect the presence of a known associated allele, a particular allele of a polymorphic locus, or the like at a specific site by looking for the presence or absence of amplification. The products of such an oligonucleotide ligation assay can also be detected by means of gel electrophoresis. Furthermore, the oligonucleotides may contain universal tags used in PCR amplification and zip code tags that are different for each allele. The zip code tags are used to isolate a specific, labeled oligonucleotide that may contain a mobility modifier (e.g. Grossman et al., 1994).

In yet another alternative, allele-specific elongation followed by ligation will form a template for PCR amplification. In such cases, elongation will occur only if there is a perfect match at the 3′ end of the allele-specific oligonucleotide using a DNA polymerase. This reaction is performed directly on the genomic DNA and the extension/ligation products are amplified by PCR. To this end, the oligonucleotides contain universal tags allowing amplification at a high multiplex level and a zip code for SNP identification. The PCR tags are designed in such a way that the two alleles of a SNP are amplified by different forward primers, each having a different dye. The zip code tags are the same for both alleles of a given SNP and they are used for hybridization of the PCR-amplified products to oligonucleotides bound to a solid support, chip, bead array or like. For an example of the procedure, see Fan et al. (Cold Spring Harbor Symposia on Quantitative Biology, Vol. LXVIII, pp. 69-78, 2003).

Another alternative includes the single-base extension/ligation assay using a molecular inversion probe, consisting of a single, long oligonucleotide (see e.g. Hardenbol et al., 2003). In such an embodiment, the oligonucleotide hybridizes on both sides of the SNP locus directly on the genomic DNA, leaving a one-base gap at the SNP locus. The gap-filling, one-base extension/ligation is performed in four tubes, each having a different dNTP. Following this reaction, the oligonucleotide is circularized whereas unreactive, linear oligonucleotides are degraded using an exonuclease such as exonuclease I of E. coli. The circular oligonucleotides are then linearized and the products are amplified and labeled using universal tags on the oligonucleotides. The original oligonucleotide also contains a SNP-specific zip code allowing hybridization to oligonucleotides bound to a solid support, chip, bead array or the like. This reaction can be performed at a highly multiplexed level.

In another alternative, the associated allele, particular allele of a polymorphic locus, or the like is scored by single-base extension (see e.g. U.S. Pat. No. 5,888,819). The template is first amplified by PCR. The extension oligonucleotide is then hybridized next to the SNP locus and the extension reaction is performed using a thermostable polymerase such as ThermoSequenase (GE Healthcare) in the presence of labeled ddNTPs. This reaction can therefore be cycled several times. The identity of the labeled ddNTP incorporated will reveal the genotype at the SNP locus. The labeled products can be detected by means of gel electrophoresis, fluorescence polarization (e.g. Chen et al., 1999) or by hybridization to oligonucleotides bound to a solid support, chip, bead array or the like. In the latter case, the extension oligonucleotide will contain a SNP-specific zip code tag.

In yet another alternative, the variant is scored by selective termination of extension. The template is first amplified by PCR and the extension oligonucleotide hybridizes in vicinity to the SNP locus, close to but not necessarily adjacent to it. The extension reaction is carried out using a thermostable polymerase such as ThermoSequenase (GE Healthcare) in the presence of a mix of dNTPs and at least one ddNTP. The latter has to terminate the extension at one of the alleles of the interrogated SNP, but not both such that the two alleles will generate extension products of different sizes. The extension product can then be detected by means of gel electrophoresis, in which case the extension products need to be labeled, or by mass spectrometry (see e.g. Storm et al., 2003).

In another alternative, the associated allele, particular allele of a polymorphic locus, or the like is detected using an invasive cleavage assay (see U.S. Pat. No. 6,090,543). There are five oligonucleotides per SNP to interrogate but these are used in a two step-reaction. During the primary reaction, three of the designed oligonucleotides are first hybridized directly to the genomic DNA. One of them is locus-specific and hybridizes up to the SNP locus (the pairing of the 3′ base at the SNP locus is not necessary). There are two allele-specific oligonucleotides that hybridize in tandem to the locus-specific probe but also contain a 5′ flap that is specific for each allele of the SNP. Depending upon hybridization of the allele-specific oligonucleotides at the base of the SNP locus, this creates a structure that is recognized by a cleavase enzyme (U.S. Pat. No. 6,090,606) and the allele-specific flap is released. During the secondary reaction, the flap fragments hybridize to a specific cassette to recreate the same structure as above except that the cleavage will release a small DNA fragment labeled with a fluorescent dye that can be detected using regular fluorescence detector. In the cassette, the emission of the dye is inhibited by a quencher.

Other types of markers can also be used for diagnostic purposes. For example, microsatellites can also be useful to detect the genetic predisposition of an individual to a given disorder. Microsatellites consist of short sequence motifs of one or a few nucleotides repeated in tandem. The most common motifs are polynucleotide runs, dinucleotide repeats (particularly the CA repeats) and trinucleotide repeats. However, other types of repeats can also be used. The microsatellites are very useful for genetic mapping because they are highly polymorphic in their length. Microsatellite markers can be typed by various means, including but not limited to DNA fragment sizing, oligonucleotide ligation assay and mass spectrometry. For example, the locus of the microsatellite is amplified by PCR and the size of the PCR fragment will be directly correlated to the length of the microsatellite repeat. The size of the PCR fragment can be detected by regular means of gel electrophoresis. The fragment can be labeled internally during PCR or by using end-labeled oligonucleotides in the PCR reaction (e.g. Mansfield et al., 1996). Alternatively, the size of the PCR fragment is determined by mass spectrometry. In such a case, however, the flanking sequences need to be eliminated. This can be achieved by ribozyme cleavage of an RNA transcript of the microsatellite repeat (Krebs et al., 2001). For example, the microsatellite locus is amplified using oligonucleotides that include a T7 promoter on one end and a ribozyme motif on the other end. Transcription of the amplified fragments will yield an RNA substrate for the ribozyme, releasing small RNA fragments that contain the repeated region. The size of the latter is determined by mass spectrometry. Alternatively, the flanking sequences are specifically degraded. This is achieved by replacing the dTTP in the PCR reaction by dUTP. The dUTP nucleosides are then removed by uracyl DNA glycosylases and the resulting abasic sites are cleaved by either abasic endonucleases such as human AP endonuclease or chemical agents such as piperidine. Bases can also be modified post-PCR by chemical agents such as dimethyl sulfate and then cleaved by other chemical agents such as piperidine (see e.g. Maxam and Gilbert, 1977; U.S. Pat. No. 5,869,242; and U.S. patent pending Ser. No. 60/335,068).

In another alternative, an oligonucleotide ligation assay can be performed. The microsatellite locus is first amplified by PCR. Then, different oligonucleotides can be submitted to ligation at the center of the repeat with a set of oligonucleotides covering all the possible lengths of the marker at a given locus (Zirvi et al., 1999). Another example of design of an oligonucleotide assay comprises the ligation of three oligonucleotides; a 5′ oligonucleotide hybridizing to the 5′ flanking sequence, a repeat oligonucleotide of the length of the shortest allele of the marker hybridizing to the repeated region and a set of 3′ oligonucleotides covering all the existing alleles hybridizing to the 3′ flanking sequence and a portion of the repeated region for all the alleles longer than the shortest one. For the shortest allele, the 3′ oligonucleotide exclusively hybridizes to the 3′ flanking sequence (U.S. Pat. No. 6,479,244).

The methods described herein may be performed, for example, by utilizing pre-packaged diagnostic kits comprising at least one probe nucleic acid selected from the SEQ ID of Tables 2, 3 and 7-10, or antibody reagent described herein, which may be conveniently used, for example, in a clinical setting to diagnose patient exhibiting symptoms or a family history of a disorder or disorder involving abnormal activity of genes from Tables 4-6.

### Method to Treat an Animal Suspected of Having Crohn's Disease

The present invention provides methods of treating a disorder associated with Crohn's disease by expressing in vivo the nucleic acids of at least one gene from Tables 4-6. These nucleic acids can be inserted into any of a number of well-known vectors for the transfection of target cells and organisms as described below. The nucleic acids are transfected into cells, ex vivo or in vivo, through the interaction of the vector and the target cell. The nucleic acids encoding a gene from Tables 4-6, under the control of a promoter, then express the encoded protein, thereby mitigating the effects of absent, partial inactivation, or abnormal expression of a gene from Tables 4-6.

Such gene therapy procedures have been used to correct acquired and inherited genetic defects, cancer, and viral infection in a number of contexts. The ability to express artificial genes in humans facilitates the prevention and/or cure of many important human disorders, including many disorders which are not amenable to treatment by other therapies (for a review of gene therapy procedures, see Anderson, 1992; Nabel & Felgner, 1993; Mitani & Caskey, 1993; Mulligan, 1993; Dillon, 1993; Miller, 1992; Van Brunt, 1998; Vigne, 1995; Kremer & Perricaudet 1995; Doerfler & Bohm 1995; and Yu et al., 1994)

Delivery of the gene or genetic material into the cell is the first critical step in gene therapy treatment of a disorder. A large number of delivery methods are well known to those of skill in the art. Preferably, the nucleic acids are administered for in vivo or ex vivo gene therapy uses. Non-viral vector delivery systems include DNA plasmids, naked nucleic acid, and nucleic acid complexed with a delivery vehicle such as a liposome. Viral vector delivery systems include DNA and RNA viruses, which have either episomal or integrated genomes after delivery to the cell. For a review of gene therapy procedures, see the references included in the above section.

The use of RNA or DNA based viral systems for the delivery of nucleic acids take advantage of highly evolved processes for targeting a virus to specific cells in the body and trafficking the viral payload to the nucleus. Viral vectors can be administered directly to patients (in vivo) or they can be used to treat cells in vitro and the modified cells are administered to patients (ex vivo). Conventional viral based systems for the delivery of nucleic acids could include retroviral, lentivirus, adenoviral, adeno-associated and herpes simplex virus vectors for gene transfer. Viral vectors are currently the most efficient and versatile method of gene transfer in target cells and tissues. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system would therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immuno deficiency virus (SIV), human immuno deficiency virus (HIV), and combinations thereof (see, e.g., Buchscher et al., 1992; Johann et al., 1992; Sommerfelt et al., 1990; Wilson et al., 1989; Miller et al., 1999; and PCT/US94/05700).

In applications where transient expression of the nucleic acid is preferred, adenoviral based systems are typically used. Adenoviral based vectors are capable of very high transduction efficiency in many cell types and do not require cell division. With such vectors, high titer and levels of expression have been obtained. This vector can be produced in large quantities in a relatively simple system. Adeno-associated virus (“AAV”) vectors are also used to transduce cells with target nucleic acids, e.g., in the in vitro production of nucleic acids and peptides, and for in vivo and ex viva gene therapy procedures (see, e.g., West et al., 1987; U.S. Pat. No. 4,797,368; WO 93/24641; Kotin, 1994; Muzyczka, 1994). Construction of recombinant AAV vectors is described in a number of publications, including U.S. Pat. No. 5,173,414; Tratschin et al., 1985; Tratschin, et al., 1984; Hermonat & Muzyczka, 1984; and Samulski et al., 1989.

In particular, numerous viral vector approaches are currently available for gene transfer in clinical trials, with retroviral vectors by far the most frequently used system. All of these viral vectors utilize approaches that involve complementation of defective vectors by genes inserted into helper cell lines to generate the transducing agent. pLASN and MFG-S are examples are retroviral vectors that have been used in clinical trials (Dunbar et al., 1995; Kohn et al., 1995; Malech et al., 1997). PA317/pLASN was the first therapeutic vector used in a gene therapy trial (Blaese et al., 1995). Transduction efficiencies of 50% or greater have been observed for MFG-S packaged vectors (Ellem et al., 1997; and Dranoff et al., 1997).

Recombinant adeno-associated virus vectors (rAAV) are a promising alternative gene delivery systems based on the defective and nonpathogenic parvovirus adeno-associated type 2 virus, All vectors are derived from a plasmid that retains only the AAV 145 by inverted terminal repeats flanking the transgene expression cassette. Efficient gene transfer and stable transgene delivery due to integration into the genomes of the transduced cell are key features for this vector system (Wagner et al., 1998, Kearns et all 996).

Replication-deficient recombinant adenoviral vectors (Ad) are predominantly used in transient expression gene therapy; because they can be produced at high titer and they readily infect a number of different cell types. Most adenovirus vectors are engineered such that a transgene replaces the Ad E1a, E1b, and E3 genes; subsequently the replication defector vector is propagated in human 293 cells that supply the deleted gene function in trans. Ad vectors can transduce multiple types of tissues in vivo, including nondividing, differentiated cells such as those found in the liver, kidney and muscle tissues. Conventional Ad vectors have a large carrying capacity. An example of the use of an Ad vector in a clinical trial involved polynucleotide therapy for antitumor immunization with intramuscular injection (Sterman et al., 1998). Additional examples of the use of adenovirus vectors for gene transfer in clinical trials include Rosenecker et al., 1996; Sterman et al., 1998; Welsh et al., 1995; Alvarez et al., 1997; Topf et al., 1998.

Packaging cells are used to form virus particles that are capable of infecting a host cell. Such cells include 293 cells, which package adenovirus, and ψ2 cells or PA317 cells, which package retrovirus. Viral vectors used in gene therapy are usually generated by a producer cell line that packages a nucleic acid vector into a viral particle. The vectors typically contain the minimal viral sequences required for packaging and subsequent integration into a host, other viral sequences being replaced by an expression cassette for the protein to be expressed. The missing viral functions are supplied in trans by the packaging cell line. For example, AAV vectors used in gene therapy typically only possess ITR sequences from the AAV genome which are required for packaging and integration into the host genome. Viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV.

In many gene therapy applications, it is desirable that the gene therapy vector be delivered with a high degree of specificity to a particular tissue type. A viral vector is typically modified to have specificity for a given cell type by expressing a ligand as a fusion protein with a viral coat protein on the viruses outer surface. The ligand is chosen to have affinity for a receptor known to be present on the cell type of interest. For example, Han et al., 1995, reported that Moloney murine leukemia virus can be modified to express human heregulin fused to gp70, and the recombinant virus infects certain human breast cancer cells expressing human epidermal growth factor receptor. This principle can be extended to other pairs of viruses expressing a ligand fusion protein and target cells expressing a receptor. For example, filamentous phage can be engineered to display antibody fragments (e.g., Fab or Fv) having specific binding affinity for virtually any chosen cellular receptor. Although the above description applies primarily to viral vectors, the same principles can be applied to nonviral vectors. Such vectors can be engineered to contain specific uptake sequences thought to favor uptake by specific target cells.

Gene therapy vectors can be delivered in vivo by administration to an individual patient, typically by systemic administration (e.g., intravenous, intraperitoneal, intramuscular, subdermal, or intracranial infusion) or topical application. Alternatively, vectors can be delivered to cells ex vivo, such as cells explanted from an individual patient (e.g., lymphocytes, bone marrow aspirates, and tissue biopsy) or universal donor hematopoietic stem cells, followed by reimplantation of the cells into a patient, usually after selection for cells which have incorporated the vector.

Ex vivo cell transfection for diagnostics, research, or for gene therapy (e.g., via re-infusion of the transfected cells into the host organism) is well known to those of skill in the art. In a preferred embodiment, cells are isolated from the subject organism, transfected with a nucleic acid (gene or cDNA), and re-infused back into the subject organism (e.g., patient). Various cell types suitable for ex vivo transfection are well known to those of skill in the art (see, e.g., Freshney et al., 1994; and the references cited therein for a discussion of how to isolate and culture cells from patients).

In one embodiment, stem cells are used in ex vivo procedures for cell transfection and gene therapy. The advantage to using stem cells is that they can be differentiated into other cell types in vitro, or can be introduced into a mammal (such as the donor of the cells) where they will engraft in the bone marrow. Methods for differentiating Crohn disease34+ cells in vitro into clinically important immune cell types using cytokines such a GM-CSF, IFN-γ and TNF-α are known (see Inaba et al., 1992).

Stem cells are isolated for transduction and differentiation using known methods. For example, stem cells are isolated from bone marrow cells by panning the bone marrow cells with antibodies which bind unwanted cells, such as Crohn disease4+ and Crohn disease8+ (T cells), Crohn disease45+ (panB cells), GR-1 (granulocytes), and lad (differentiated antigen presenting cells).

Vectors (e.g., retroviruses, adenoviruses, liposomes, etc.) containing therapeutic nucleic acids can be also administered directly to the organism for transduction of cells in vivo. Alternatively, naked DNA can be administered.

Administration is by any of the routes normally used for introducing a molecule into ultimate contact with blood or tissue cells, as described above. The nucleic acids from Tables 4-6 are administered in any suitable manner, preferably with the pharmaceutically acceptable carriers described above. Suitable methods of administering such nucleic acids are available and well known to those of skill in the art, and, although more than one route can be used to administer a particular composition, a particular route can often provide a more immediate and more effective reaction than another route (see Samulski et al., 1989). The present invention is not limited to any method of administering such nucleic acids, but preferentially uses the methods described herein.

The present invention further provides other methods of treating Crohn's disease such as administering to an individual having Crohn's disease an effective amount of an agent that regulates the expression, activity or physical state of at least one gene from Tables 4-6. An “effective amount” of an agent is an amount that modulates a level of expression or activity of a gene from Tables 4-6, in a cell in the individual at least about 10%, at least about 20%, at least about 30%, at least about 40%, at least about 50%, at least about 60%, at least about 70%, at least about 80% or more, compared to a level of the respective gene from Tables 4-6 in a cell in the individual in the absence of the compound. The preventive or therapeutic agents of the present invention may be administered, either orally or parenterally, systemically or locally. For example, intravenous injection such as drip infusion, intramuscular injection, intraperitoneal injection, subcutaneous injection, suppositories, intestinal lavage, oral enteric coated tablets, and the like can be selected, and the method of administration may be chosen, as appropriate, depending on the age and the conditions of the patient. The effective dosage is chosen from the range of 0.01 mg to 100 mg per kg of body weight per administration. Alternatively, the dosage in the range of 1 to 1000 mg, preferably 5 to 50 mg per patient may be chosen. The therapeutic efficacy of the treatment may be monitored by observing various parts of the GI tract, by endoscopy, barium, colonoscopy, or any other monitoring methods known in the art. Other ways of monitoring efficacy can be, but are not limited to monitoring inflammatory conditions involving the upper gastrointestinal tract such as monitoring the amelioration on the esophageal discomfort, decrease in pain, improved swallowing, reduced chest pain, decreased heartburn, decreased regurgitation of solids or liquids after swallowing or eating, decrease in vomiting, or improvement in weight gain or improvement in vitality.

The present invention further provides a method of treating an individual clinically diagnosed with Crohns' disease. The methods generally comprises analyzing a biological sample that includes a cell, in some cases, a GI track cell, from an individual clinically diagnosed with Crohn's disease for the presence of modified levels of expression of at least 1 gene, at least 10 genes, or at least 50 genes from Tables 4-6. A treatment plan that is most effective for individuals clinically diagnosed as having a condition associated with Crohn's disease is then selected on the basis of the detected expression of such genes in a cell. Treatment may include administering a composition that includes an agent that modulates the expression or activity of a protein from Tables 4-6 in the cell. Information obtained as described in the methods above can also be used to predict the response of the individual to a particular agent. Thus, the invention further provides a method for predicting a patient's likelihood to respond to a drug treatment for a condition associated with Crohn's disease, comprising determining whether modified levels of a gene from Tables 4-6 is present in a cell, wherein the presence of protein is predictive of the patient's likelihood to respond to a drug treatment for the condition. Examples of the prevention or improvement of symptoms accompanied by Crohn's disease that can monitored for effectiveness include prevention or improvement of diarrhea, prevention or improvement of weight loss, inhibition of bowel tissue edema, inhibition of cell infiltration, inhibition of surviving period shortening, and the like, and as a result, a preventing or improving agent for diarrhea, a preventing or improving agent for weight loss, an inhibitor for bowel tissues edema, an inhibitor for cell infiltration, an inhibitor for surviving period shortening, and the like can be identified.

The invention also provides a method of predicting a response to therapy in a subject having Crohn's disease by determining the presence or absence in the subject of one or more markers associated with Crohn's disease described in Tables 2, 3 and 7-10, diagnosing the subject in which the one or more markers are present as having Crohn's disease, and predicting a response to a therapy based on the diagnosis e.g., response to therapy may include an efficacious response and/or one or more adverse events. The invention also provides a method of optimizing therapy in a subject having Crohn's disease by determining the presence or absence in the subject of one or more markers associated with a clinical subtype of Crohn's disease, diagnosing the subject in which the one or more markers are present as having a particular clinical subtype of Crohn's disease, and treating the subject having a particular clinical subtype of Crohn's disease based on the diagnosis. As an example, treatment for the fibrostenotic subtype of Crohn's disease currently includes surgical removal of the affected, structured part of the bowel.

Thus, while there are a number of treatments for Crohn's disease currently available, they all are accompanied by various side effects, high costs, and long complicated treatment protocols, which are often not available and effective in a large number of individuals. Accordingly, there remains a need in the art for more effective and otherwise improved methods for treating and preventing Crohn's disease. Thus, there is a continuing need in the medical arts for genetic markers of Crohn's disease and guidance for the use of such markers. The present invention fulfills this need and provides further related advantages.

### EXAMPLES

### Example 1

### GWS Using Samples from the QFP

Recruited Samples from the Quebec Founder Population

All individuals were sampled from the Quebec founder population (QFP). Membership in the founder population was defined as having four grandparents with French Canadian family names who were born in the Province of Quebec, Canada or in adjacent areas of the Provinces of New Brunswick and Ontario or in New England or New York State. The Quebec founder population has two distinct advantages over general populations for LD mapping. Because it is relatively young (about 12 to 15 generations from the mid 17th century to the present) and because it has a limited but sufficient number of founders (approximately 2600 effective founders, Charbonneau et al., 1987), the Quebec population is characterized both by extended LD and by decreased genetic heterogeneity. The increased extent of LD allows the detection of disease associated genes using a reasonable marker density, while still allowing the increased meiotic resolution of population-based mapping. The number of founders is small enough to result in increased LD and reduced allelic heterogeneity, yet large enough to insure that all of the major disease genes involved in general populations are present in Quebec. Reduced allelic heterogeneity will act to increase relative risk imparted by the remaining alleles and so increase the power of case/control studies to detect genes and gene alleles involved in complex disorders within the Quebec population. The specific combination of age in generations, optimal number of founders and large present population size makes the QFP optimal for LD-based gene mapping.

Patient inclusion criteria for the study include diagnosis for Crohn's disease by any one of the following: a colonoscopy, a radiological examination with barium, an abdominal surgical operation or a biopsy or a surgical specimen. The colonoscopy diagnosis consists of observing linear, deep or serpentigenous ulcers, pseudopolyps, or skip areas. The barium radiological examination consists of the detection of strictures, ulcerations and string signs by observing the barium enema and the small bowel followed through an NMRI series.

Patients that were diagnosed with ulcerative colitis, infectious colitis or other intestinal diseases were excluded from the study. All human sampling was subject to ethical review procedures.

All enrolled QFP subjects (patients and controls) provided a 30 ml blood sample (3 barcoded tubes of 10 ml). Samples were processed immediately upon arrival at Genizon's laboratory. All samples were scanned and logged into a LabVantage Laboratory Information Management System (LIMS), which served as a hub between the clinical data management system and the genetic analysis system. Following centrifugation, the buffy coat containing the white blood cells was isolated from each tube. Genomic DNA was extracted from the buffy coat from one of the tubes, and stored at 4° C. until required for genotyping. DNA extraction was performed with a commercial kit using a guanidine hydrochloride based method (FlexiGene, Qiagen) according to the manufacturer's instructions. The extraction method yielded high molecular weight DNA, and the quality of every DNA sample was verified by agarose gel electrophoresis. Genomic DNA appeared on the gel as a large band of very high molecular weight. The remaining two buffy coats were stored at −80° C. as backups.

The QFP samples were collected as family trios consisting of Crohn's disease subjects and two first degree relatives. Of the 500 trios, 477 were Parent, Parent, Child (PPC) trios; the remainders were Parent, Child, Child (PCC) trios. Only the PPC trios were used for the analysis reported here because they produced equal numbers of more accurately estimated case and control haplotypes than the PCC trios. 382 trios were used in the genome wide scan component of the study. One member of each trio was affected with Crohn's disease. For the 382 trios used in the genome wide scan, these included 189 daughters, 90 sons, 54 mothers and 49 fathers. When a child was the affected member of the trio, the two non-transmitted parental chromosomes (one from each parent) were used as controls, when one of the parents was affected, that person's spouse provided the control chromosomes. The recruitment of trios allowed a more precise determination of long extended haplotypes.

### Genome Wide Scan Genotyping

Genotyping was performed using Perlegen's ultra-high-throughput platform. Marker loci were amplified by PCR and hybridized to wafers containing arrays of oligonucleotides. Allele discrimination was performed through allele-specific hybridization. In total, 248,535 SNPs, distributed as evenly as possible throughout the genome, were genotyped on the 382 QFP trios for a total of 372,802,500 genotypes. These markers were mostly selected from various databases including the ˜1.6 million SNP database of Perlegen Life Sciences (Patil, 2001); several thousand were obtained from the HapMap consortium database and/or dbSNP at NCBI. The SNPs were chosen to maximize uniformity of genetic coverage and to cover a distribution of allele frequencies. All SNPs that did not pass the quality controls for the assay, that is, that had a minor allele frequency of less than 1%, a Mendelian error rate within trios greater than 1%, that deviated significantly from the Hardy-Weinberg equilibrium, or that had excessive missing data (cut-off at 5% missing values or higher) were removed from the analysis. Genetic analysis was performed on a total of 165,785 SNPs (158,775 autosomal, 6869 X chromosome and 141 Y chromosome). The average gap size was approximately 17 kb. Of the 165,785 markers, ˜140,000 had a minor allele frequency (MAF) greater than 10% for the QFP. The genotyping information was entered into a Unified Genotype Database (a proprietary database under development) from which it was accessed using custom-built programs for export to the genetic analysis pipeline. Analyses of these genotypes were performed with the statistical tools described in the section below. The GWS permitted the identification of the candidate chromosomal region linked to Crohn's disease (Table 1).

### Genetic Analysis

1. Dataset Quality Assessment

Prior to performing any analysis, the dataset from the GWS was verified for completeness of the trios. The program GGFileMod removed any trios with abnormal family structure or missing individuals (e.g. trios without a proband, duos, singletons, etc.), and calculated the total number of complete trios in the dataset. The trios were also tested to make sure that no subjects within the cohort were related more closely than second cousins (6 meiotic steps).

Subsequently, the program DataCheck2.1 was used to calculate the following statistics per marker and per family:
Minor allele frequency (MAF) for each marker; Missing values for each marker and family; Hardy Weinberg Equilibrium for each marker; and
Mendelian segregation error rate.

The following acceptance criteria were applied for internal analysis purposes:
MAF>1%;
Missing values <1%;
Observed non-Mendelian segregation <0.33%;
Non significant deviation in allele frequencies from Hardy Weinberg equilibrium.

Markers or families not meeting these criteria were removed from the dataset in the following step. Analyses of variance were performed using the algorithm GenAnova, to assess whether families or markers have a greater effect on missing values and/or non-Mendelian segregation. This was used to determine the smallest number of data points to remove from the dataset in order to meet the requirements for missing values and non-Mendelian segregation. The families and/or markers were removed from the dataset using the program DataPull, which generates an output file that is used for subsequent analysis of the genotype data.

2. Phase Determination

The program PhaseFinderSNP2.0 was used to determine phase from trio data on a marker-by-marker, trio-by-trio basis. The output file contains haplotype data for all trio members, with ambiguities present when all trio members are heterozygous or where data is missing. The program FileWriterTemp was then used to determine case and control haplotypes and to prepare the data in the proper input format for the next stage of analysis, using the expectation maximization algorithm, PL-EM, to call phase on the remaining ambiguities. This stage consists of several modules for resolution of the remaining phase ambiguities. PLEMInOut1 was first used to recode the haplotypes for input into the PL-EM algorithm in 15-marker blocks for the genome wide scan data and for 11 marker blocks for fine and ultra-fine mapping data sets. The haplotype information was encoded as genotypes, allowing for the entry of known phase into the algorithm; this method limits the possible number of estimated haplotypes conditioned on already known phase assignments. The PL-EM algorithm was used to estimate haplotypes from the “pseudo-genotype” data in 11 or 15-marker windows, advancing in increments of one marker across the chromosome. The results were then converted into multiple haplotype files using the program PLEMInOut2. Subsequently PLEMBlockGroup was used to convert the individual 11 or 15-marker block files into one continuous block of haplotypes for the entire chromosome, and to generate files for further analysis by LDSTATS and SINGLETYPE. PLEMBlockGroup takes the consensus estimation of the allele call at each marker over all separate estimations (most markers are estimated 11-15 different times as the 11 or 15 marker blocks pass over their position).

3. Haplotype Association Analysis

Haplotype association analysis was performed using the program LDSTATS. LDSTATS tests for association of haplotypes with the disease phenotype. The algorithms LDSTATS (v2.0) and LDSTATS (v4.0) define haplotypes using multi-marker windows that advance across the marker map in one-marker increments. Windows can contain any odd number of markers specified as a parameter of the algorithm. Other marker windows can also be used. At each position the frequency of haplotypes in cases and controls was calculated and a chi-square statistic was calculated from case control frequency tables. For LDSTATS v2.0, the significance of the chi-square for single marker and 3-marker windows was calculated as Pearson's chi-square with degrees of freedom. Larger windows of multi-allelic haplotype association were tested using Smith's normalization of the square root of Pearson's Chi-square. In addition, LDSTATS v2.0 calculates Chi-square values for the transmission disequilibrium test (TDT) for single markers in situations where the trios consisted of parents and an affected child.

LDSTATS v4.0 calculates significance of chi-square values using a permutation test in which case-control status is randomly permuted until 350 permuted chi-square values are observed that are greater than or equal to chi-square value of the actual data. The P value is then calculated as 350/the number of permutations required.

Table 2 lists the results for association analysis using LDSTATs (v2.0 and v4.0) for the region described above based on the genome wide scan genotype data for 382 QFP trios. For each region that was associated with Crohn disease in the genome wide scan, we report in Table 3 the allele frequencies and the relative risk (RR) for the haplotypes contributing to the best signal at each SNP in the region. The best signal at a given location was determined by comparing the significance (p-value) of the association with Crohn disease for window sizes of 1, 3, 5, 7, and 9 SNPs, and selecting the most significant window. For a given window size at a given location, the association with Crohn disease was evaluated by comparing the overall distribution of haplotypes in the cases with the overall distribution of haplotypes in the controls. Haplotypes with a relative risk greater than one increase the risk of developing Crohn disease while haplotypes with a relative risk less than one are protective and decrease the risk.

4. Singletype Analysis

The SINGLETYPE algorithm assesses the significance of case-control association for single markers using the genotype data from the laboratory as input in contrast to LDSTATS single marker window analyses, in which case-control alleles for single markers from estimated haplotypes in file, hapatctr.txt, as input. SINGLETYPE calculates P values for association for both alleles, 1 and 2, as well as for genotypes, 11, 12, and 22, and plots these as −log₁₀ P values for significance of association against marker position.

Gene Identification and Characterization from GWS on the QFP Samples

A series of gene characterization was performed for candidate region 1 described in Table 1. Any gene or EST mapping to the interval based on public map data or proprietary map data was considered as a candidate Crohn's disease gene (see Tables 4-6 for the list of genes).

### Example 2

### The Replication and Functional Characterization of ATG16L1 Gene in European Samples

### DETAILED DESCRIPTION OF THE DRAWINGS

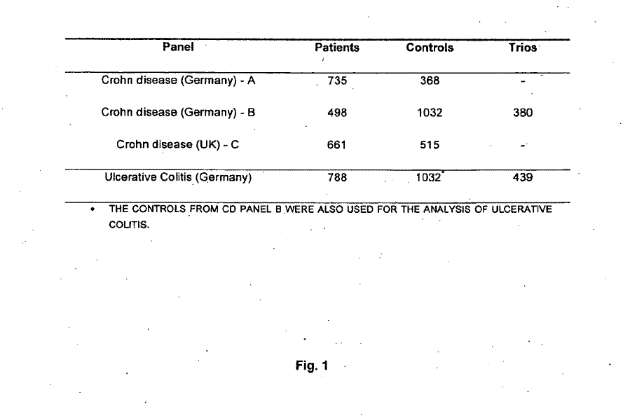

FIG. 1 Crohn disease (CD) patient and control samples used for association analysis. The patient samples are organized in ‘panels’ that correspond to successive steps of the study. Index cases from trios were also used in the case-control analyses so that, for example, a total of 878 cases (498+380) were available for the case-control comparison in panel B.

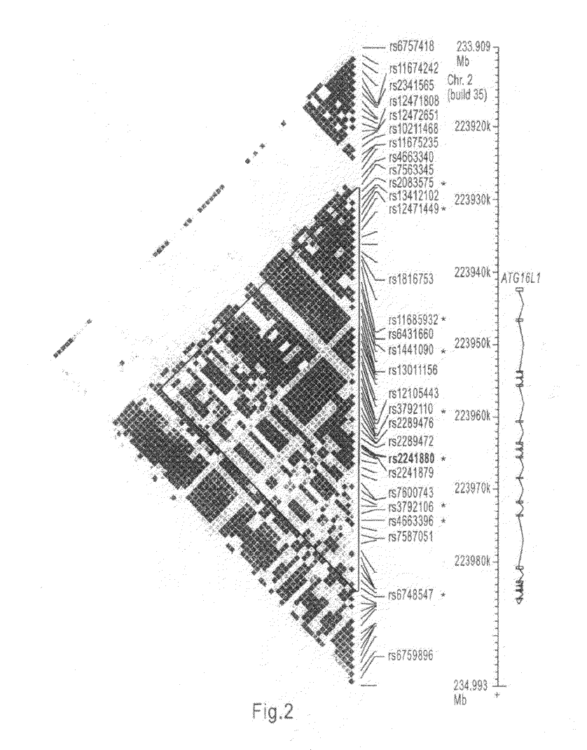

FIG. 2 Overview of the physical and genetic structure of the ATG16L1 gene region. The physical position of the SNPs investigated and a schematic chart of the gene structure are shown in the top panel. The only coding SNP is marked in red. The coordinates refer to the genome assembly build 35. The lower panel gives an overview of the linkage disequilibrium structure of the locus (D') as generated by Haploview from the Caucasian HapMap data. The SNPs used in the haplotype analysis (see also Table 10) are marked with asterisks.

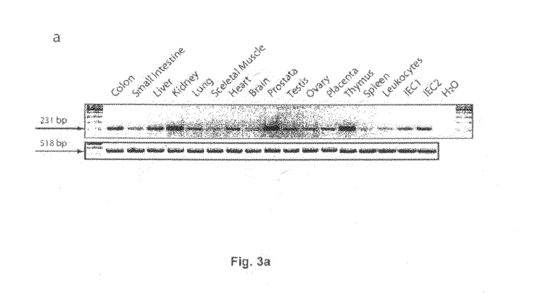

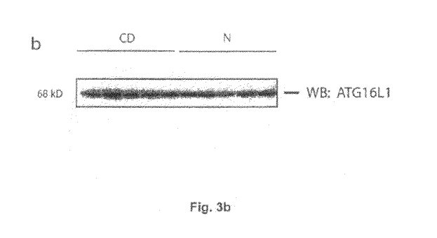

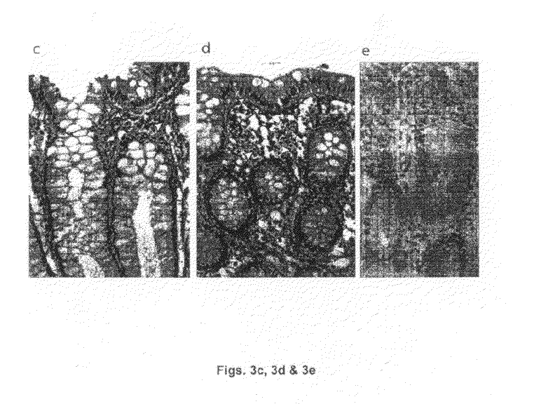

FIG. 3 Presence of ATG16L1 in tissues of interest. Panel A shows the expression of ATG16L1 in a set of different tissues as detected by RT-PCR (IEC, Intestinal Epithelial Cells). The corresponding □-Actin control (518 by amplicon size) is given below. Panel B shows a Western blot analysis of ATG16L1 in colonic mucosa. Proteins (15 μg) from rectal mucosal biopsies of Crohn disease patients (CD) and normal controls (N) were separated by denaturing SDS-PAGE, transferred onto PVDF membranes and probed for presence of ATG16L1 using a specific primary antibody and horseradish-peroxidase-coupled secondary antibody. ATG16L1 is present in the mucosa of CD patients and healthy controls at the same level. Panels C-E demonstrate the expression and localization of the ATG16L1 protein in colonic tissue from a CD patient (C) and a normal control (D). Intestinal epithelial cells are marked with arrows, mononuclear cells are highlighted with arrowheads. Panel E shows a control staining without the primary antibody in a CD sample.

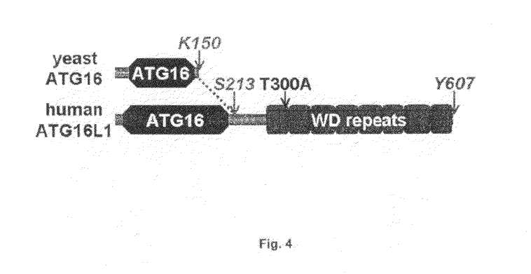

FIG. 4 Domain architecture of human ATG16L1 and yeast ATG16. The position of the variant amino acid T300A in the WD repeat domain, consequent to SNP rs2241880, is marked. The annotated APG16 Pfam domain consists of coiled coils. The C-terminal residue K150 of yeast ATG16 corresponds to S213 of human ATG16L1 according to a pair-wise sequence alignment.

FIG. 53D structure model of the WD-repeat domain of human ATG16L1. The 32 β-strands forming an 8-bladed β-propeller are numbered as in Supplementary FIG. 1. The location of the variant amino acid T300A in strand β3, corresponding to rs2241880, is marked in yellow.

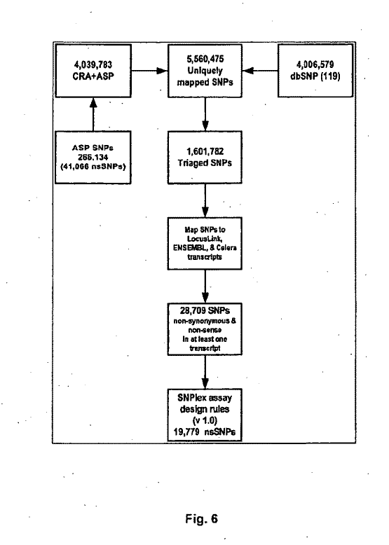

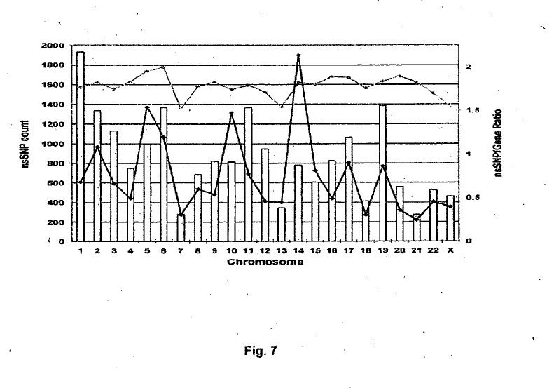

FIG. 6: SNP selection and assay development process

FIG. 7: Distribution of nεSNP panel across human chromosomes

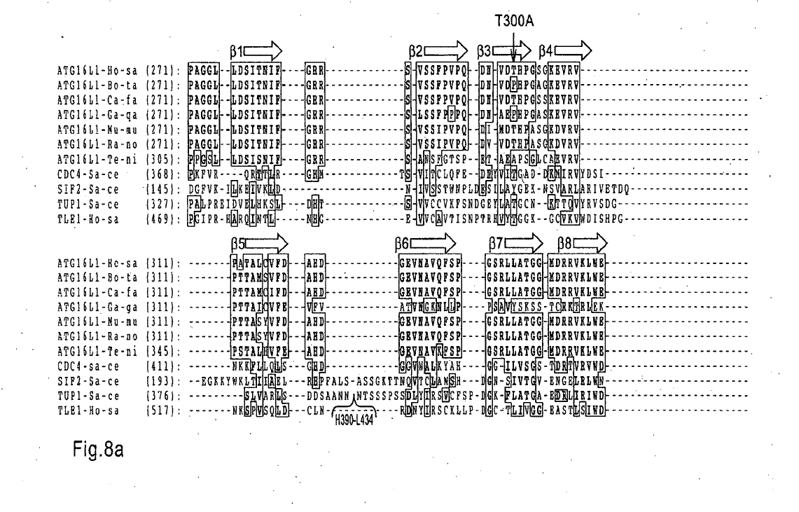

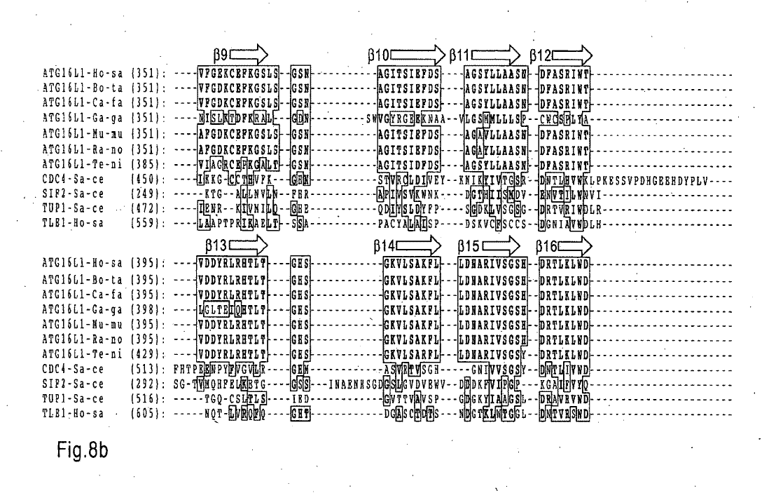

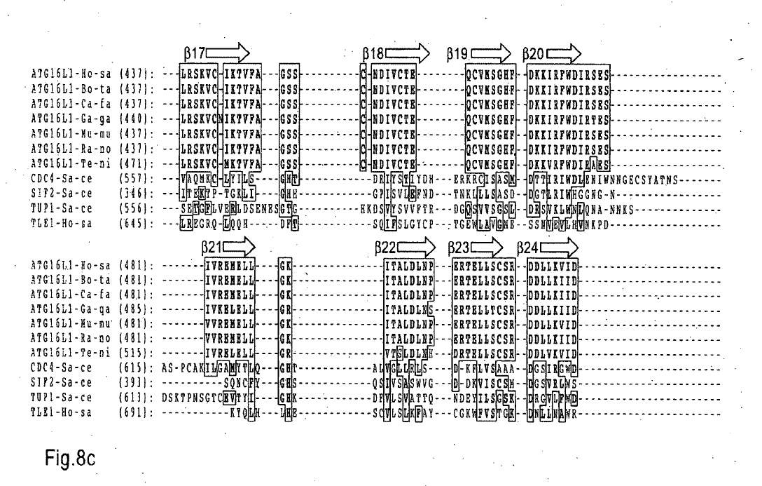

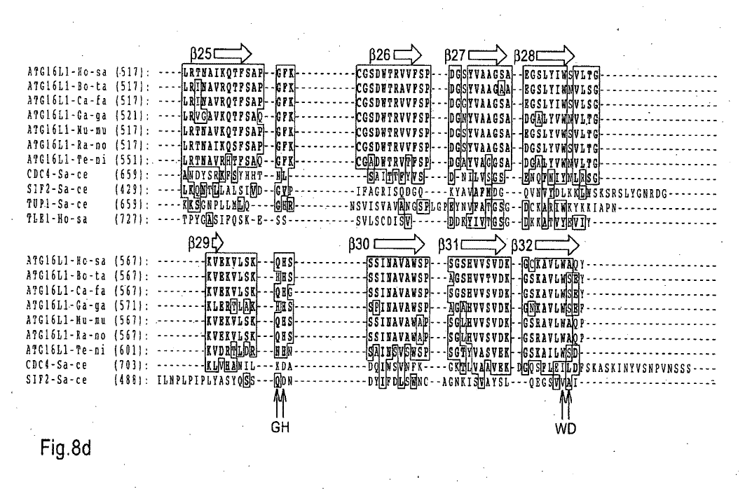

FIG. 8 (a-d): Structure-based multiple sequence alignment of the WD-repeat domains of ATG16L1 homologs and the related proteins CDC4, SIF2, TUP1, and TLE1 with known 3D structures. Each alignment row contains a single WD repeat frequently characterized by GH and WD dipeptides (bottom annotation). CDC4 and SIF2 comprise eight WD repeats, whereas TUP1 and TLE1 contain seven WD repeats. The secondary structure depicted at the top of each alignment rows is taken from CDC4 and represents the □-strands characteristic of WD repeats, Physicochemically conserved amino acids are highlighted in blue boxes. Residue numbering in the alignment is based on complete protein sequences. The position of the sequence variant T300A of human ATG16L1 is marked (top annotation).

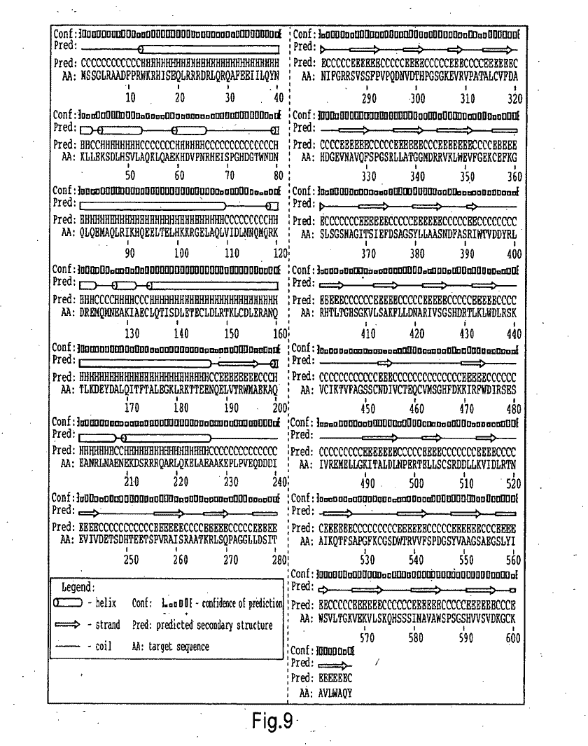

FIG. 9: Exemplary output of a secondary structure prediction for human ATG16L1 (protein accession number Q676U5). Here, the PSIPRED web server produced the depicted prediction.

### PATIENT RECRUITMENT

The German patients in panels A and B were recruited at the Charité University Hospital (Berlin, Germany) and the Department of General Internal Medicine of the Christian-Albrechts-University (Kiel, Germany), with the support of the German Crohn and Colitis Foundation (see FIG. 1). Clinical, radiological and endoscopic (i.e. type and distribution of lesions) examinations were required to unequivocally confirm the diagnosis of Crohn disease, and histological findings also had to be confirmative of, or compatible with, the diagnosis. In case of uncertainty, patients were excluded from the study. The patient sample has been used in several studies before and the respective publications provide a more detailed account of the phenotyping techniques employed. German control individuals were obtained from the POPGEN biobank. The UK patients were recruited by the collaborating centre as described before; UK controls were obtained from the 1958 British Birth Cohort (http://www.b58cgene.sgul.ac.uk). All recruitment protocols were approved by ethics committees at the participating centres prior to commencement of the study and participants were obliged to give written, informed consent.

### Construction of the Coding SNP Set

Full details regarding the construction of the panel of 19,779 non-synonymous (or ‘coding’) SNPs (cSNPs) of the invention are described below. In brief, SNPs from dbSNP (build 117) were combined with polymorphisms discovered by the Applera exon resequencing project or during the shotgun sequencing of the human genome by Celera Genomics. Variants that could be unequivocally mapped to the human genome assembly (Celera R27) were then further selected based on their observed or expected (i.e. “double-hit” SNPs) heterozygosity in populations of European and African descent. Putatively functional SNPs were then defined as those non-synonymous variants that altered the amino-acid sequence of an annotated NCBl RefSeq, Celera or ENSEMBL transcript. For the resulting 28,709 cSNPs, DNA sequence context and allele information was submitted to the assay design pipeline of the SNPlex™ Genotyping System (v. 1.0 pre-release). The sequence context was masked for adjacent double-hit SNPs to avoid probes overlapping with other common SNPs. Finally a total of 19,779 SNPlex assay designs were obtained that were manufactured and partitioned in 428 multiplex pools of up to 48 SNPs each (mean; 45 SNPs per assay pool).

1. SNP Database for Marker Selection

SNP data were obtained from three sources: (1) the Celera Human RefSNP database, version 3.4, which included about 2.4 million SNPs discovered during the shotgun sequencing of the Human genome by Celera Genomics, as well as 2.2 million imported from public sources, mainly dbSNP, JSNP, and HGMD; (2) The Applera Corp. SNP Project (ASP) database, which consists of 266,135 SNPs discovered in 20 European Americans and 19 African Americans by Sanger sequencing of PCR amplicons overlapping the exons of 23,363 genes annotated by Celera Genomics; and (3) SNPs included in NCBI's dbSNP database, release 117. All SNPs were mapped to the Celera Human genome assembly Release 27 and only those that mapped to unique locations, after removing redundancy, were advanced in the process (see Table A below).

TABLE A
SNP database for marker selection

| Source | Number of SNPs |
|---|---|
| Celera RefSNP database | 4,039,783 |
| Applera SNP Project database | 266,135 |
| NCBI dbSNP release 117 | 4,006,579 |
| Total non-redundant, uniquely | 5,560,475 |
| mapped | |

2. SNP Selection and Assay Development

The SNP selection process was aimed at developing a comprehensive list of common putative functional cSNPs from all possible sources, and to avoid putative SNPs that are rare variants or potential sequencing or analysis artifacts. We thus triaged SNPs based on their measured or expected heterozygosity in populations of European and African descent. For this we used the allele frequency data obtained during the ASP project and from the genotyping of 177,781 SNPs in about 45 samples each of European American, African American, with TaqMan® Validated SNP Genotyping Assays. When no allele frequency information in population panels was available, we looked for evidence of independent discovery (so called “double-hit” SNPs). We used as evidence the ASP project calls, the donor information of the Celera shotgun reads, and the dbSNP submission handles. SNPs whose minor alleles were observed in at least two distinct donors in either the ASP or Celera shotgun SNP discovery were selected. We identified SNPs discovered independently by Celera or ASP and by the public SNP discovery efforts. We also compared single-donor Celera SNPs to the NCBI human genomic assembly to find cases where the Celera minor allele was confirmed in the public consensus sequence. Finally, for SNPs when the single source was dbSNP, SNPs with at least 3 distinct submission handles were included. We compiled 1,601,782 SNPs that meet these requirements. We then identified non-synonymous cSNPs (nsSNPs), either missense or nonsense, by mapping these SNPs to Celera, RefSeq/LocusLink, and ENSEMBL transcripts. At the end of the process we obtained 28,709 nsSNPs in at least one transcript to be submitted for assay design (see FIG. 6).

To avoid designing oligoprobes overlapping other common SNPs, we “masked” the context sequence of target SNPs for other adjacent SNPs using only the list of triaged SNPs described above. Masked context sequence and allele information was submitted to the assay design pipeline of the SNPlex™ Genotyping System, version 1.0, in batches no larger than 2500 SNPs. Design batches were organized to group SNPs belonging to a candidate gene list related to immunity and inflammation, and by similar molecular function as predicted by the PANTHER protein classification, when possible. After removing SNP assays that failed to meet the oligoprobe design or genome specificity rules, we obtained and manufactured 19,779 SNPlex SNP genotyping assays distributed in 440 multiplexes with probes to type up to 48 SNPs each. Please refer to Table A for details on the distribution and annotations of all SNPs included in the SNPlex multiplex pools.

3. Distribution and Features of nsSNPs Panel

The figure below (FIG. 7) shows the distribution of the set of nsSNPs in our final panel across the human chromosomes. The bars show the actual number of nsSNP per chromosome, and the lines represent the nsSNP/gene ratio, based on the Celera gene annotation (R27). The raw SNP to gene ratio (blue line) shows an apparent higher than average (0.75 nsSNP/gene) value for chromosomes 5, 10, and 14, whereas chromosomes 7, 18, 20, 21, and X show a lower SNP to gene ratio. However, this apparent distortion disappears if we normalize to count only genes with at least one nsSNPs in our set (green line), where the genome average is 1.78 nsSNP/gene. The total number of Celera genes covered by nsSNP in our panel is 9,672 (out of 25,030 genes in the Celera R27 annotation).

The distribution of genes was then analyzed with nsSNPs using the PANTHER protein function classification¹⁶. Of interest was to ascertain if particular functional classes are unrepresented (i.e. genes classes where common nonsynonymous SNPs are rare) or overrepresented (i.e. gene classes with frequent common variation). A binomial distribution test was used to calculate p-value for the observed vs. expected category representation as compared to the entire gene complement, as per the Celera human gene annotation, Release 27. The analysis shown in Table B shows that certain molecular function categories are over- or underrepresented in our panel with statistical significance (note that a large proportion of genes are not currently classified).

TABLE B
Gene representation of nsSNP panel

| | hCGR27 | nsSNP | Expected | Over (+) | |
|---|---|---|---|---|---|
| Category | (n = 25030) | (n = 9603) | on nsSNP | Under (−) | p-value |
| Protein biosynthesis | 837 | 180 | 321.12 | | 2.10E−18 |
| Pre-mRNA processing | 226 | 51 | 86.71 | | 2.16E−05 |
| Chromatin packaging and remodeling | 185 | 43 | 70.98 | | 2.28E−04 |
| Protein folding | 164 | 38 | 62.92 | | 4.73E−04 |
| Cell adhesion | 533 | 313 | 204.49 | | 6.18E−18 |
| Olfaction | 296 | 192 | 113.56 | | 9.44E−12 |
| Chemosensory perception | 328 | 202 | 125.84 | | 1.94E−10 |
| Signal transduction | 3387 | 1507 | 1299.46 | | 7.20E−10 |
| Cell surface receptor mediated signal transduction | 1601 | 748 | 614.24 | | 3.49E−08 |
| Cell adhesion-mediated signaling | 292 | 173 | 112.03 | | 4.65E−08 |
| Sensory perception | 588 | 307 | 225.59 | | 1.09E−07 |
| Cell structure and motility | 1064 | 502 | 408.21 | | 2.45E−06 |
| Proteolysis | 734 | 360 | 281.61 | | 2.93E−06 |
| Cell structure | 679 | 334 | 260.5 | | 5.22E−06 |
| G-protein mediated signaling | 897 | 425 | 344.14 | | 9.83E−06 |
| Cell communication | 1250 | 567 | 479.57 | | 3.58E−05 |
| Extracellular matrix protein-mediated signaling | 54 | 40 | 20.72 | | 1.08E−04 |
| Lipid, fatty acid and steroid metabolism | 678 | 317 | 260.12 | | 2.92E−04 |

Underrepresented in this panel are classes of genes known to be highly conserved and that carry out several fundamental cellular processes (e.g. protein synthesis, chromatin packaging genes), whereas overrepresented gene classes include some classes that are know for presenting higher levels of genetic variation (e.g. olfactory receptors, cell surface/adhesion). This suggest that selection pressure might limit common potentially deleterious polymorphisms in highly conserved genes participating in fundamental cellular processes, whereas at the other extreme selection may favour common functional variation on certain classes of genes that deal with environmental interactions and other functions.

### Genotyping and Sequencing

Genomic DNA was prepared at participating centres using a variety of methods, DNA samples were thus evaluated by gel electrophoresis for the presence of high-molecular weight DNA and adjusted to 20-30 ng/μl DNA content using the Picogreen fluorescent dye (Molecular Probes—Invitrogen, Carlsbad, Calif., USA). One microliter of genomic DNA was amplified by the GenomiPhi (Amersham, Uppsala, Sweden) whole genome amplification system and fragmented at 99° C. for five minutes. One hundred nanograms of DNA were dried overnight in TwinTec hardshell 384well plates (Eppendorf, Hamburg, Germany) at room temperature. Genotyping was performed with these plates using the SNPlex™ Genotyping System (Applied Biosystems, Foster City, Calif., USA) on an automated platform, employing TECAN Freedom EVO and 96-well and 384-well TEMO liquid handling robots (TECAN, Mannedorf, Switzerland). Genotypes for the cSNP screening experiment in patient panel A were generated by automatic calling using the Genemapper 4.0 software (Applied Biosystems) with the following settings: sigma separation ≧6, angle separation for 2 cluster SNPs≦1.2 radians, median cluster intensity ≧2.2 logs. For all significant markers in panel A and all replication studies, genotypes were additionally reviewed manually and call rates >90% required. All process data were logged into, and administered by, a database-driven LIMS. Unless noted otherwise, all genotypes were generated through SNPlex.

TaqMan® SNP Genotyping Assays (Applied Biosystems, Foster City, Calif., USA) were used to genotype the CARD15 variants as described and to genotype rs2241880 in the German and UK samples by way of a technology-independent replication on an automated platform. Sequencing of genomic DNA was performed using Applied Biosystems BigDye™ chemistry according to the supplier's recommendations. Traces were inspected for the presence of SNPs and InDels using InSNP and novoSNP.

1. Coding SNP Scan and Replication

A total of 19,779 coding SNPs were genotyped in the samples of panel A (735 CD patients and 368 controls from Northern Germany, Table 7) using the SNPlex™ system. Genotyping was successful for 16,360 assays, as defined by a mean fluorescence reading greater than 500 units on the ABI 3730×1 sequencer. Of the workable SNPs, 7,159 occurred at a minor allele frequency greater 1% and were thus included in the subsequent analyses. The markers were first ranked and prioritized for follow-up on the basis of the p-values obtained in the single-locus allele-based and genotype-based test for disease association in panel A. A p-value of 0.01 in the allele-based test was used as a cut-off for inclusion in a replication study, which resulted in 72 putative disease variants that were also evaluated in panel B (380 German CD trios, 941 single patients and 1046 independent controls, Table 7). When p<0.05 in both the TDT and the case-control comparison was held to indicate formal replication, only three markers, rs2241880 (Thr300Ala) in the ATG16L1 gene and the two previously reported variants rs1050152 (Leu503Phe) in the SLC22A4 (OCTN1) gene and rs2066845 (‘SNP12’) in the CARD15 (NOD2) gene were found to match this criterion. The newly found association of the G allele at rs2241880 with CD was significant with p=1.6×10⁻⁵ in the allele-based case-control comparison and with p=2.7×10⁻⁵ in the TDT. Thus, all subsequent mapping and replication efforts were confined to this variant. Genotyping results obtained with the SNPlex™ system were confirmed using a TaqMan® assay (99.8% genotype concordance), thus excluding artefacts due to technological problems.

2. Follow-Up of ATG16L1 Mutation Detection and Linkage Disequilibrium Analysis

For a more comprehensive assessment of the CD risk conferred by changes in the ATG16L1 gene, a systematic search for additional mutations was carried out by re-sequencing all exons, splice sites and the promoter region of 47 CD patients. Apart from rs2241880, no further coding or splice site variants could be identified in this experiment. The CD risk associated with ATG16L1 variation was then also analysed at the haplotype level, using 28 tagging SNPs selected from the Caucasian HapMap data on the basis of r²>0.8 and a minor allele frequency greater than 1%. The localisation of the respective SNPs and their LD structure is shown in FIG. 2. When the tagging SNPs were genotyped in panel B (Table 9), the intronic SNP rs2289472 was found to have the same minor allele frequency (0.47) as coding SNP rs2241880, and to yield a slightly more significant disease association (p=1.4×10⁻⁵). This variant is localized 1082 bases upstream of exon 9 and is not located in any recognizable regulatory motif. Synonymous SNP rs13011156, on the other hand, was not found to be significantly associated with CD. In a logistic regression model, none of the tagging SNPs significantly improved the model fit in the presence of rs2241880 (all p>0.05). Together with the results of a subsequent haplotype analysis (Table 10), these findings imply that the CD risk conferred by ATG16L1 gene variation is indeed mainly due to carriership of susceptibility allele G at rs2241880.

The disease association of rs2242880 was also replicated in a UK-derived CD sample (panel C: n_{cases}=515, n_{controls}=661), using an independent TaqMan assay. The British data yielded p=0.0004 in the allele-based comparison (OR: 1.35; 95% CI: 1.14-1.59) and p=0.0001 in the genotype-based test (OR for carriership of G: 1,70; 95% CI: 1.22-2.36). In the combined analysis of all German individuals (panels A and B), the odds ratio was 1.45 (95% CI: 1.21-1.74) for heterozygous carriership of G and 1.77 (95% CI: 1.43-2.18) for homozygosity. The combined p-values for the German samples were p=4×10⁻⁸ for the allele-based and p=2×10⁻⁷ for the genotype-based test.

3. Evaluation of rs2241880 in Ulcerative Colitis

CD-associated SNP rs2241880 was also evaluated in a sample of German patients with ulcerative colitis (UC sample, Table 7). Allele frequencies of G in cases (0.46) and controls (0.47) were virtually identical, and evidence for association was thus neither obtained from the case-control comparison (p>0.4 in both the allele- and genotype-based test) nor from the TDT (p>0.9).

### Statistical Analysis

All markers were tested for possible deviations from Hardy-Weinberg equilibrium in controls before inclusion in the association analyses. Single marker association tests and transmission disequilibrium tests (TDT) were performed using Haploview and GENOMIZER. In families with multiple affected individuals, one trio was randomly extracted for TDT analysis. Haplotype frequency estimates were obtained from singletons using an implementation of the EM algorithm (COCAPHASE). Significance testing of haplotype frequency differences was also performed with COCAPHASE and TDTPHASE, making use of the fact that twice the log-likelihood ratio between two nested data models approximately follows a χ² distribution with k degrees of freedom, where k is the difference in parameter number between the two models. Significance assessment of associations was performed using χ² or Fisher's exact test for contingency tables, as appropriate. Genotype-based logistic regression analysis was performed with R (www.r-project.org), coding individual SNP genotypes as categorical variables. Analysis of statistical interactions between risk genotypes, including Breslow-Day tests for odds ratio homogeneity, was done by means of procedure FREQ of the SAS/STAT® software package (Cary, N.C., USA).

1. Statistical Interaction Between rs2241880 and CARD15

The ATG16L1 gene encodes a protein in the autophagosome pathway that processes intracellular bacteria. Since both the ATG16L1 and the CARD15 protein are involved in the innate defence against bacterial pathogens, the disease-associated variants in the two genes were investigated for a possible statistical interaction with respect to CD risk. To this end, individuals in the German fine mapping and replication sample (panel B) were classified as either homozygous wild-type (dd), heterozygous carrier (dD) or homozygous carrier (DD, which included compound heterozygotes) for the three main causative CARD15 SNPs rs2066844 (R702W), rs2066845 (G908R) and rs2066847 (L1007fs). Appropriateness of this classification is supported by the published haplotype structure of the CARD15 gene. The frequency and odds ratio for individual CARD15 risk genotypes, stratified by rs2241880 genotype, are shown in Table 8. A statistical interaction clearly became apparent between rs2241880 and the CARD15 low-risk genotypes dd and Dd. Thus, carriership of rs2241880 allele G was found to be a risk factor for CD only in the presence of CARD15 genotype dd, but not dD. However, the odds ratio difference was only significant for rs2241880 genotype GG (2.03 versus 1.04; Breslow-Day χ²=4.267, 1 d.f., p=0.039), and not for AG. On the background of CARD15 high-risk genotype DD, the risk conferred by carriership of the rs2241880 allele G appeared to be higher than in the presence of dd or Dd, but the confidence intervals of the respective odds ratios were still wide owing to the small number of DD controls (Table 9). Nevertheless, when rs2241880 genotypes GG and AG were combined, the joint OR of 5.89 (95% CI: 1.23-29.21) was found to be statistically significantly larger than unity (Fisher's exact two-sided p=0.016), thereby confirming that rs2241880 allele G is a risk factor on a high-risk CARD15 genotype background as well

### In Silico Protein Analysis

Aligned sequences were retrieved from the UniProt and Ensembl databases (www.uniprot.org and www.ensembl.org) and protein domain architectures from the Pfam database (www.sanger.ac.uk/Software/Pfam/). To predict the 3D structure of the ATG16L1 gene product, we explored the fold recognition results returned by the BioInfoBank online meta-server and the FFAS03 and Arby web servers. Based upon their very similar predictions, a sequence-structure alignment of ATG16L1 to the crystal structure of yeast CDC4 (FIG. 8) was constructed for the 3D-modeling server WHAT IF (http://swift.cmbi.kun.nl/WIWWWI/), which returned a structural model of the ATG16L1 WD-repeat domain.

We retrieved protein sequences from the UniProt (http://www.uniprot.org) and Ensembl (http://www.ensembl.org) databases. Protein domain architectures were taken from the Pfam database (http://www.sanger.ac.uk/Software/Pfam/). 3D crystal structure coordinates were obtained from the Protein Data Bank (http://www.pdb.org/) and corresponding domain definitions from the SCOP database (http://scop.mrc-lmb.cam.ac.uk/scop/index.html). Ensembl identifiers, UniProt accession numbers, and PDB codes are listed in Figure C-E, respectively.

Using the alignment program MUSCLE (http://www.drive5.com/muscle/), we computed multiple sequence alignments of ATG16L1 homologs contained in the Ensembl family ATG16L1 (identifier ENSF00000001431) and the Pfam family APG16 (accession number PF08614). To analyze the alignments further, we included evolutionarily related WD-repeat proteins with known 3D structures: yeast cell division control protein 4 (CDC4), yeast SIR4-interacting protein 2 (SIR2), yeast glucose repression regulatory protein 1 (TUP1), and human transducin-like enhancer protein 1 (TLE1). The alignments were manually refined based on multiple sequence alignments of the WD40 Pfam family (accession number PF00400), the published WD-repeat consensus sequence, and structure superpositions of WD-repeat proteins using FATCAT (http://fatcat.ljcrf.edu/). The alignment figures were prepared and illustrated using the editors GeneDoc (http://www.psc.edu/biomed/genedoc/), Jalview (http://www.jalview.org), and SeaView (http://pbil.univ-lyon1.fr/software/seaview.html).

The secondary structure assignment to PDB structures was obtained from the DSSP database (http://www.cmbi.kun.nl/gv/dssp/). To predict the secondary structure of ATG16L1 homologs in different species, we contacted the prediction servers PSIPRED (http://bioinf.cs.ucl.ac.uk/psipred/), YASPIN (http://ibivu.cs.vu.nl/programs/yaspinwww/), PROFsec (http://www.predictprotein.org), and Porter (http://distill.ucd.ie/porter/). We also formed consensus predictions by majority voting. All servers consistently predicted β-strands characteristic of eight WD repeats in ATG16L1, and a helical linker may precede the eighth WD-repeat as it is the case for CDC4.

To predict the three-dimensional WD-repeat domain structure of human ATG16L1, we investigated the fold recognition results returned by the BioInfoBank online meta-server (http://bioinfo.pl/meta/) and compared them to the very similar predictions by the web servers FFAS03 (http://ffas.ljcrf.edu) and Arby (http://arby.bioinf.mpi-inf.mpg.de/arby/jsp/index.jsp). The BioInfoBank server contacts a dozen other structure prediction servers and is coupled to a 3D-Jury system that assesses the quality of the returned results based on a sophisticated scoring scheme. FFAS03 and ARBY also provide statistically derived confidence scores for structure predictions. In agreement with the WD40 Pfam family classification, all servers predicted at least seven WD repeats at the C-terminus of ATG16L1 starting near residue P311. In addition, the secondary structure predictions for human ATG16L1 and its species homologs as well as the conservation of amino acids characteristic of WD repeats indicated an eighth non-canonical WD repeat in the 40-residue region P271-V310.

Since diverged WD repeats have already been observed with other WD-repeat proteins such as CDC4, SIF2, and coronin-1, we chose the eight-bladed β-propeller structure of the WD-repeat domain from the CDC4 subunit of the yeast SCF ubiquitin ligase complex as structural template for ATG16L1. Because the WD-repeat domain of ATG16L1 is replaced by an actin domain in the ATG16L1 homolog of Ustilago maydis (UniProt accession number Q4P303), one may speculate that the WD-repeat domain of human ATG16L1 is involved in actin regulation like coronin proteins with a domain architecture similar to ATG16L1. To model the 3D protein structure of ATG16L1, we extracted a pairwise sequence-structure alignment from the manually curated multiple alignment of ATG16L1 and WD-repeat domain homologs and submitted it to the WHAT IF server (http://swift.cmbi.kun.nl/WIWWWI/. The image of the resulting full-atom protein structure model was illustrated using Yasara (http://www.yasara.org) and POV-Ray (http://www.povray.org).

### Functional Studies

1. Isolation of Primary Epithelial Cells

Epithelial cell preparation was carried out using a standard protocol as described. In brief, mucosal biopsies were placed in 1.5 mM EDTA in Hanks balanced salt solution without calcium and magnesium (HBSS) and tumbled for 10 minutes at 37° C. The supernatant containing debris and mainly villus cells was discarded. The mucosa was incubated again with HBSS/EDTA for 10 minutes at 37° C. The supernatant was collected into a 15 ml tube. The remaining mucosa was shortly vortexed in PBS and this supernatant was also collected. It contained complete crypts, some single cells, and a small amount of debris. To separate IECs (crypts) from contaminating non-epithelial cells, the suspension was allowed to sediment for 15 minutes. The cells (mainly complete crypts) were collected and washed twice with PBS. The number and viability of the cells were determined by trypan blue exclusion. The purity of the epithelial cell preparation was checked by routine hematoxylin-eosin staining, showing more than 90% of epithelial cells.

2. mRNA Isolation and RT-PCR

Total RNA from primary intestinal epithelial cells was isolated using the RNeasy kit from Qiagen. Some 300 ng of total RNA were reverse transcribed as described elsewhere. For investigation of tissue specific expression patterns, a commercial tissue panel was obtained from Clontech (Palo Alto, Calif., USA). Primers used for amplification of APG16L are listed in Table D (expected amplicon length: 231 bp). The following conditions were applied: denaturation for 5 min at 95° C.; 25 cycles of 30 sec at 95° C., 20 sec at 60° C., 45 sec at 72° C.; final extension for 10 min at 72° C. To confirm the use of equal amounts of RNA in each experiment, all samples were checked in parallel for β-actin mRNA expression. All amplified DNA fragments were analyzed on 1% agarose gels and subsequently documented by a BioDoc Analyzer (Biometra, Göttingen, Germany).

3. Western Blot

Biopsies from five healthy controls without any obvious intestinal pathology and from five Crohn patients with confirmed ileal and colonic inflammation were lysed and subjected to Western blot analysis as described in Waetzig et al. 10 μg of total protein were separated by SDS polyacrylamide gel electrophoresis and transferred to PVDF membrane by standard techniques. APG16L was detected using a polyclonal anti-APG16 antibody and horseradish-peroxidase (HRP)-coupled secondary antibody.

4. Immunohistochemistry

Paraformaldehyde-fixed paraffin-embedded biopsies from normal controls (n=5) and from patients with confirmed colonic Crohn disease (n=5) were analysed which were obtained in parallel from the same sites as the biopsies used for the expression analysis studies. Two slides of each biopsy were stained with hematoxylin-eosin for routine histological evaluation. The other slides were subjected to a citrate-based antigen retrieval procedure, permeabilized by incubation with 0.1% Triton X-100 in 0.1M phosphate-buffered saline (PBS), washed three times in PBS and blocked with 0.75% bovine serum albumin in PBS for 20 minutes. Sections were subsequently incubated with the primary antibody (anti-APG16L, ABGENT, San Diego, Calif.) at a 1:200 dilution in 0.75% BSA for 1 h at room temperature. After washing in PBS, tissue bound antibody was detected using biotinylated goat-anti rabbit (Vector Laboratory, Burlingame, Calif.) followed by HRP-conjugated avidin, both diluted at 1:100 in PBS. Controls were included using irrelevant primary antibodies as well as omitting the primary antibodies using only secondary antibodies and/or HRP-conjugated avidin. No significant staining was observed with any of these controls (data not shown). Bound antibody was detected by standard chromogen technique (Vector Laboratory) and visualized by an Axiophot microscope (Zeiss, Jena, Germany). Pictures were captured by a digital camera system (Axiocam, Zeiss).

5. Expression and Localization of ATG16L1

Expression of the ATG16L1 gene was investigated by RT-PCR in a panel of different tissues, confirming expression in colon, small bowel, intestinal epithelial cells, and immune tissues like spleen and leukocytes (FIG. 3, panel A). Recently, the existence of multiple splice variants of ATG16L1 was reported and many splice variants are annotated in the Golden Path assembly (http://genome.ucsc.edu). In all annotated and reported splice variants, exon 9, which contains CD susceptibility variant rs2241880, is translated in the same reading frame, thus consistently leading to a Thr to Ala amino acid substitution by the SNP. In a Western Blot from colon tissue (FIG. 3, panel B), a dominant 68.2 kD protein band was identified corresponding to the annotated coding sequence AY398617 (protein accession number Q676U5). This protein sequence was therefore used for the modelling of the ATG16L1 protein (see below). Expression of ATG16L1 in the intestinal epithelium was shown by immunohistochemistry (FIG. 3, panel C) and no significant difference in expression level was detected between normal and patient tissue.

6. Location of T300A in ATG16L1

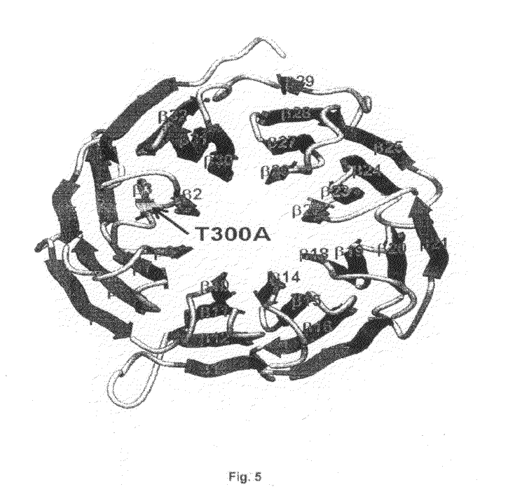

ATG16L1 homologues are present in a wide range of eukaryotes in the same domain architecture, except for yeast ATG16 (FIG. 4). The threonine residue at position 300, which is substituted to alanine by rs2241880, is conserved across many species including mouse and rat, suggesting an important functional role of this amino acid. Human ATG16L1 is organized into an N-terminal APG16 domain consisting of coiled coils and eight C-terminal WD repeats. The 3D structure of ATG16L1 was modelled using the eight-bladed β-propeller crystal structure of the evolutionarily related WD-repeat domain in yeast CDC4 (Supplementary protein analysis methods). The location of the T300A variant in human ATG16L1 corresponds to T397 of CDC4, where it lies at the N-terminus of the WD-repeat domain in the β3 strand of the first propeller blade (FIG. 5 and FIG. 6). Therefore, the Thr to Ala amino acid change encoded by rs2241880 might have a detrimental effect on the structural stability of the affected blade and on potential binding sites nearby.

TABLE C

Primer sequences used for the
mutation detection of the ATG16L1 gene.

| | | | |
|---|---|---|---|
| Region | Primer | Sequence | Amplicon |

| | | | |
|---|---|---|---|
| Promoter | ATG16L_p2_F | 5′-CACGAAAAGCAGCTTAACAATCAAAG-3′ | 828 bp |
| | ATG16L_p2_R | 5′-AGTGACGCCAGCCTGTAGCC-3′ | |
| | ATG16L_p1_F | 5′-CACAGTGCTGACTGCATTACATGG-3′ | 829 bp |
| | ATG16L_p1_R | 5′-GCCTCAGGTTCCCGCTGAC-3′ | |
| Exon 01 | ATG16L_e01_F | 5′-TCCGGCCCTCTCGAAAATC-3′ | 505 bp |
| | ATG16L_e01_R | 5′-GGGAAAATCCTCCAAAGATAAAACG-3′ | |
| Exon 02 | ATG16L_e02_F | 5′-GGGAAGACATTCTTGCAGGTG-3′ | 536 bp |
| | ATG16L_e02_R | 5′-TGAATCCTGGCAGGTTAGATGAG-3′ | |
| Exon 03 | ATG16L_e03_F | 5′-CTGCTGGAGACACCCGAATG-3′ | 445 bp |
| | ATG16L_e03_R | 5′-TGGTGATGGGCCTCAATCTG-3′ | |
| Exon 04 | ATG16L_e04-2_F | 5′-TGGCAGGGATAGTTCCCCTTTG-3′ | 397 bp |
| | ATG16L_e04-2_R | 5′-GCTGGTAGAAAAGGATCCCAGAGTG-3′ | |
| Exon 05 | ATG16L_e05_F | 5′-TTTCCTCTCCTAATGGATTATCCTG-3′ | 600 bp |
| | ATG16L_e05_R | 5′-TTGTGGTGTATTTCCTTTTTCTAACTC-3′ | |
| Exon 06 | ATG16L_e06_F | 5′-TGATGTTATGAGTTTCGGCTTGTG-3′ | 388 bp |
| | ATG16L_e06_R | 5′-CATTAGAAGCTATGATCACACCACTGC-3′ | |
| Exon 07 | ATG16L_e07_F | 5′-TGGCAGCTCTTCCTTTTTCTCC-3′ | 433 bp |
| | ATG16L_e07_R | 5′-TGCTTCCCTCCCATTAAGCAG-3′ | |
| Exon 08 | ATG16L_e08_F | 5′-AGGCTGGGTTTTCCCTTTCC-3′ | 437 bp |
| | ATG16L_e08_R | 5′-GCACGCAGCGAGATTAAGAGG-3′ | |
| Exon 09 | ATG16L_e09_F | 5′-CTCATTTGAGTGAGGGTGCTTTTG-3′ | 537 bp |
| | ATG16L_e09_R | 5′-CCATCCCTCATGCTAGCAATCC-3′ | |
| Exon 10 | ATG16L_e10_F | 5′-AGAATCTTAGTTGACCTGGGCTAGGAG-3′ | 433 bp |
| | ATG16L_e10_R | 5′-TGCTCAAACGATCCCTTACATAAAATG-3′ | |
| Exon 11 | ATG16L_e11_F | 5′-′TCATGTTCTCTTTGTCCTGCTATTTTG-3′ | 427 bp |
| | ATG16L_e11_R | 5′-GCAGAACCCAAGGGTTTATCAGAG-3′ | |
| Exon 12 | ATG16L_e12_F | 5′-GCGAGTTGAAGCACACTCACG-3′ | 392 bp |
| | ATG16L_e12_R | 5′-GGAAACACAGATTTCCCCAAGG-3′ | |
| Exon 13 | ATG16L_e13-14_F | 5′-GAGTCACTGTGCCTGACCTGTTTC-3′ | 548 bp |
| | ATG16L_e13-14_R | 5′-CAAGCAGAGGCACCAACGTG-3′ | |
| Exon 14 | ATG16L_e15-2_F | 5′-GGCTTCATGTTTAGAGGGGCACTG-3′ | 427 bp |
| | ATG16L_e15-2_R | 5′-TTCATGGGAAAGAACAGCCAAGTG-3′ | |
| Exon 15 | ATG16L_e16_F | 5′-TGTCTTAGGGTCTGTTGATGGGAAAG-3′ | 515 bp |
| | ATG16L_e16_R | 5′-GGGGGTGGGTCACTACTAACCTG-3′ | |
| Exon 16 | ATG16L_e17-2_F | 5-CCTGAGCTGCTCCCGTGATG-3′ | 385 bp |
| | ATG16L_e17-2_R | 5′-CAATAATGGTGGCCTGCAATTATGAAC-3′ | |
| Exon 17 | ATG16L_e18_F | 5′-CGGACGGGGCTGAAATACTG-3′ | 456 bp |
| | ATG16L_e18_R | 5′-AGTGGCCCCAGCTTCTCTCC-3′ | |
| Exon 18 | ATG16L_e19_F | 5′-AGTGAGCTCCTGCCTTGTCG-3′ | 407 bp |
| | ATG16L_e19_R | 5′-CCCATTCACGGCAAAGCTAC-3′ | |

TABLE D

Primer sequences used for the amplification
of the ATG16L1 transcript (Exon 10, 11, and
12 exist in all splice variants) in the RT-PCR.

| | | | |
|---|---|---|---|
| Region | Primer | Sequence | Amplicon |

| | | | |
|---|---|---|---|
| Exon 10-12 | ATG1GL10-12_F | 5′-AACGCTGTGCAGTTCAGTCCAG-3′ | 231 bp |
| | ATG16L10-12_R | 5′-AGTGACGCCAGCCTGTAGCC-3′ | |

TABLE E
Ensembl/UniProt identifiers for ATG16L1 homologs and related WD-repeat
proteins shown in FIG. S1. PDB codes are given for the WD-repeat domain
structures CDC4, SIR2, TUP1, and TLE1.

| Protein | Species | Alignment | UniProt/Ensembl | PDB |
|---|---|---|---|---|
| ATG16L1 | Homo sapiens | ATG16L1-Ho-sa | Q676U5 | — |
| ATG16L1 | Bos taurus | ATG16L1-Bo-ta | ENSBTAP00000005140 | — |
| ATG16L1 | Canis familiaris | ATG16L1-Ca-fa | ENSCAFP00000017340 | — |
| ATG16L1 | Gallus gallus | ATG16L1-Ga-ga | ENSGALP00000002472 | — |
| ATG16L1 | Mus musculus | ATG16L1-Mu-mu | Q8C0J2 | — |
| ATG16L1 | Rattus norwegicus | ATG16L1-Ra-no | ENSRNOP00000024445 | — |
| ATG16L1 | Tetraodon | ATG16L1-Te-ni | Q4SB59 | — |
| | nigroviridis | | | |
| CDC4 | Saccharomyces | CDC4-Sa-ce | P07834 | 1nex, |
| | cerevisiae | | | chain B |
| SIF2 | Saccharomyces | SIF2-Sa-ce | P38262 | 1r5m, |
| | cerevisiae | | | chain A |
| TUP1 | Saccharomyces | TUP1-Sa-ce | P16649 | 1erj, |
| | cerevisiae | | | chain A |
| TLE1 | Homo sapiens | TLE1-Ho-sa | Q04724 | 1gxr, |
| | | | | chain A |

All publications, patents and patent applications mentioned in the specification and reference list are herein incorporated by reference in their entirety for all purposes. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in molecular biology, genetics, or related fields are intended to be within the scope of the following claims.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2nd Ed., ed. by Sambrook, Fritsch and Maniatis (Cold Spring Harbor Laboratory Press: 1989); DNA Cloning, Volumes I and H (D. N. Glover ed., 4); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Haines & S. J. Higgins eds. 1984); Culture Of Animal Cells (R. 1. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

The sequence listing and Tables 1-10 submitted herewith are herein incorporated by reference in their entireties and are considered to be part of the application as filed

### REFERENCES

1. Shivananda, S. et al. Incidence of inflammatory bowel disease across Europe: is there a difference between north and south? Results of the European Collaborative Study on Inflammatory Bowel Disease (EC-IBD). Gut 39, 690-697 (1996).
2. Probert, C. S., Jayanthi, V., Rampton, D. S. & Mayberry, J. F. Epidemiology of inflammatory bowel disease in different ethnic and religious groups: limitations and aetiological clues. Int J Colorectal Dis 11, 25-28 (1996).
3. Podolsky, D. K. Inflammatory Bowel Disease. N Engl J Med 325, 928-937 (1991).
4. Orholm, M. et al. Familial occurrence of inflammatory bowel disease. N Engl J Med 324, 84-88 (1991).
5. Kuster, W., Pascoe, L., Purrmann, J., Funk, S. & Majewski, F. The genetics of Crohn disease: complex segregation analysis of a family study with 265 patients with Crohn disease and 5,387 relatives. Am J Med Genet 32, 105-108 (1989).
6. Tysk, C., Lindberg, E., Jarnerot, G. & Floderus Myrhed, B. Ulcerative colitis and Crohn's disease in an unselected population of monozygotic and dizygotic twins. A study of heritability and the influence of smoking. Gut 29, 990-996 (1988).
7. Thompson, N. P., Driscoll, R., Pounder, R. E. & Wakefield, A. J. Genetics versus environment in inflammatory bowel disease: results of a British twin study. BMJ 312, 95-96 (1996).
8. Satsangi, J., Rosenberg, W. M. C. & Jewell, D. P. The prevalence of Inflammatory Bowel Disease in relatives of patients with Crohn's disease. Eur J Gastroenterol Hepatol 6, 413-416 (1994).
9. Probert, C. S. et al. Prevalence and family risk of ulcerative colitis and Crohn's disease: an epidemiological study among Europeans and south Asians in Leicestershire. Gut 34, 1547-1551 (1993).
10. Meucci, G. et al. Familial aggregation of inflammatory bowel disease in northern Italy: a multicenter study. The Gruppo di Studio per le Malattie Infiammatorie Intestinali (IBD Study Group). Gastroenterology 103, 514-519 (1992).
11. Hugot, J. P. et al. Association of NOD2 leucine-rich repeat variants with susceptibility to Crohn's disease. Nature 411, 599-603 (2001).
12. Ogura, Y. et al. A frameshift mutation in NOD2 associated with susceptibility to Crohn's disease. Nature 411, 603-606 (2001).
13. Rioux, J. D. et al, Genetic variation in the 5q31 cytokine gene cluster confers susceptibility to Crohn disease. Nat Genet 29, 223-228. (2001).
14. Peltekova, V. D. et al. Functional variants of OCTN cation transporter genes are associated with Crohn disease. Nat Genet 36, 471-475 (2004).
15. Stoll, M. et al. Genetic variation in DLG5 is associated with inflammatory bowel disease. Nat Genet 36, 476-480 (2004).
16. Brant, S. R. et al. MDR1 Ala893 polymorphism is associated with inflammatory bowel disease. Am J Hum Genet 73, 1282-1292 (2003).
17. Ho, G. T. et al. ABCB1/MDR1 gene determines susceptibility and phenotype in ulcerative colitis: discrimination of critical variants using a gene-wide haplotype tagging approach. Hum Mol Genet 15, 797-805 (2006).
18. Schwab, M. et al., Association between the C3435T MDR1 gene polymorphism and susceptibility for ulcerative colitis. Gastroenterology 124, 26-33 (2003).
19. Yamazaki, K. et al. Single nucleotide polymorphisms in TNFSF15 confer susceptibility to Crohn's disease. Hum Mol Genet (2005).
20. Maeda, S. et al. Nod2 mutation in Crohn's disease potentiates NF-kappaB activity and IL-1 beta processing. Science 307, 734-738 (2005).
21. Kobayashi, K. S. et al. Nod2-dependent regulation of innate and adaptive immunity in the intestinal tract. Science 307, 731-734 (2005).
22. Girardin, S. E. et al. Nod2 is a general sensor of peptidoglycan through muramyl dipeptide (MDP) detection. J Biol Chem 278, 8869-8872 (2003).
23. Hampe, J. et al. Association between insertion mutation in NOD2 gene and Crohn's disease in German and British populations. Lancet 357, 1925-1928. (2001).
24. Marks, D. J. et al. Defective acute inflammation in Crohn's disease: a clinical investigation. Lancet 367, 668-678 (2005).
25. Rosenstiel, P. et al. TNF-alpha and IFN-gamma regulate the expression of the NOD2 (CARD15) gene in human intestinal epithelial cells. Gastroenterology 124, 1001-1009 (2003).
26. Herbert, A. et al. A common genetic variant is associated with adult and childhood obesity. Science 312, 279-283 (2006).
27. Smyth, D. J. et al. A genome-wide association study of nonsynonymous SNPs identifies a type 1 diabetes locus in the interferon-induced helicase (IFIH1) region. Nat Genet (2006).
28. Croucher, P. J. P. et al. Haplotype structure and association to Crohn's disease of CARD15 mutations in two ethnically divergent populations. Eur J Hum Genet, in press (2003).
29. Zheng, H. et al. Cloning and analysis of human Apg16L. DNA Seq 15, 303-305 (2004).
30. Orlicky, S., Tang, X., Willems, A., Tyers, M. & Sicheri, F. Structural basis for phosphodependent substrate selection and orientation by the SCF^{Cdc4} ubiquitin ligase. Cell 112, 243-256 (2003).
31. Reich, D. E. & Lander, E. S. On the allelic spectrum of human disease. Trends Genet 17, 502-510 (2001).
32. Inohara, N. et al., Nod1, an Apaf-1-like activator of caspase-9 and nuclear factor-kappaB. J Biol Chem 274, 14560-14567 (1999).
33. Chamaillard, M. et al. An essential role for NOD1 in host recognition of bacterial peptidoglycan containing diaminopimelic acid. Nat Immunol 4, 702-707 (2003).
34. Codogno, P. & Meijer, A. J. Autophagy and signaling: their role in cell survival and cell death. Cell Death Differ 12 Suppl 2, 1509-1518 (2005).
35. Mizushima, N. The pleiotropic role of autophagy: from protein metabolism to bactericide. Cell Death Differ 12 Suppl 2, 1535-1541 (2005).
36. Deretic, V. Autophagy in innate and adaptive immunity. Trends Immunol 26, 523-528 (2005).
37. Swanson, M. S. & Molofsky, A. B. Autophagy and inflammatory cell death, partners of innate immunity. Autophagy 1, 174-176 (2005).
38. Kirkegaard, K., Taylor, M. P. & Jackson, W. T. Cellular autophagy: surrender, avoidance and subversion by microorganisms. Nat Rev Microbiol 2, 301-314 (2004).
39. Ogawa, M. et al. Escape of intracellular Shigella from autophagy. Science 307, 727-731 (2005).
40. Mizushima, N. et al. Mouse Apg16L, a novel WD-repeat protein, targets to the autophagic isolation membrane with the Apg12-Apg5 conjugate. J Cell Sci 116, 1679-1688 (2003).
41. Kuma, A., Mizushima, N., Ishihara, N. & Ohsumi, Y. Formation of the approximately 350-kDa Apg12-Apg5 Apg16 multimeric complex, mediated by Apg16 oligomerization, is essential for autophagy in yeast. J Biol Chem 277, 18619-18625 (2002).
42. Mizushima, N., Yoshimori, T. & Ohsumi, Y. Role of the Apg12 conjugation system in mammalian autophagy. Int J Biochem Cell Biol 35, 553-561 (2003).
43. Li, D. & Roberts, R. WD-repeat proteins: structure characteristics, biological function, and their involvement in human diseases. Cell Mol Life Sci 58, 2085-2097 (2001).
44. Schreiber, S., Rosenstiel, P., Albrecht, M., Hampe, J. & Krawczak, M. Genetics of Crohn disease, an archetypal inflammatory barrier disease. Nat Rev Genet 6, 376-388 (2005).
45. Ott, S. J. et al. Reduction in diversity of the colonic mucosa associated bacterial microflora in patients with active inflammatory bowel disease. Gut 53, 685-693 (2004).
46. Swidsinski, A. et al. Mucosal flora in inflammatory bowel disease. Gastroenterology 122, 44-54 (2002).
47. Lennard-Jones, J. E. Classification of inflammatory bowel disease. Scand J Gastroenterol Suppl 170, 2-6 (1989).
48. Truelove, S. C. & Pena, A. S. Course and prognosis of Crohn's disease. Gut 17, 192-201 (1976).
49. Curran, M. E. et al. Genetic Analysis of Inflammatory Bowel Disease in a Large European Cohort Supports Linkage to Chromosomes 12 and 16. Gastroenterology 115, 1066-1071 (1998).
50. Hampe, J. et al. A genome-wide analysis provides evidence for novel linkages in Inflammatory Bowel Disease in a large European cohort. Am J Hum Genet 64, 808-816 (1999).
51. Hampe, J. et al. The interferon gamma gene as a positional and functional candidate gene for inflammatory bowel disease. Intl J Colorectal Dis 13, 260-263 (1998).
52. Krawczak, M., Nikolaus, S., von Eberstein, H., El Mokhtari, N. E. & Schreiber, S. PopGen: Population-based recruitment of patients and controls for the analysis of complex genotype-phenotype relationships. Community Genet 9, 55-61 (2006).
53. Onnie, C. M. et al. Associations of allelic variants of the multidrug resistance gene (ABCB1 or MDR1) and inflammatory bowel disease and their effects on disease behavior: a case-control and meta-analysis study. Inflamm Bowel Dis 12, 263-271 (2006).
54. Venter, J. C. et al., The sequence of the human genome. Science 291, 1304-1351 (2001).
55. Tobler, A. R. et al. The SNPlex genotyping system: a flexible and scalable platform for SNP genotyping. J Biomol Tech 16, 398-406 (2005).
56. Hampe, J. et al., An integrated system for high throughput TaqMan based SNP genotyping. Bioinformatics 17, 654-655. (2001).
57. Hampe, J. et al. Evidence for a NOD2-independent susceptibility locus for inflammatory bowel disease on chromosome 16p. Proc Natl Acad Sci USA 99, 321-326. (2002).
58. Manaster, C. et al. InSNP: a tool for automated detection and visualization of SNPs and InDels. Hum Mutat 26, 11-19 (2005).
59. Weckx, S. et al. novoSNP, a novel computational tool for sequence variation discovery. Genome Res 15, 436-442 (2005).
60. Barrett, J. C., Fry, B., Mailer, J. & Daly, M. J. Haploview: analysis and visualization of LD and haplotype maps. Bioinformatics 21, 263-265 (2005).
61. Franke, A. et al. GENOMIZER: an integrated analysis system for genome-wide association data. Hum Mutat 27, 583-588 (2006).
62. Dudbridge, F. Pedigree disequilibrium tests for multilocus haplotypes. Genet Epidemiol 25, 115-121 (2003).
63. Waetzig, G. H. et al. Soluble tumor necrosis factor (TNF) receptor-1 induces apoptosis via reverse TNF signaling and autocrine transforming growth factor-beta1. Faseb J 19, 91-93 (2005).
Kerlavage, A. et al. The Celera Discovery System. Nucleic Acids Res 30, 129-36 (2002).
Venter, J. C. et al. The sequence of the human genome. Science 291, 1304-51 (2001).
Sherry, S. T. et al. dbSNP: the NCBl database of genetic variation. Nucleic Acids Res 29, 308-11 (2001).
Hirakawa, M. et al. JSNP: a database of common gene variations in the Japanese population. Nucleic Acids Res 30, 158-62 (2002).
Stenson, P. D. et al. Human Gene Mutation Database (HGMD): 2003 update. Hum Mutat 21, 577-81 (2003).
Adams, M. et al. Applied genomics: exploring functional variation and gene expression. Am. J. Hum. Genet. 71, 203 (2002).
Bustamante, C. D. et al. Natural selection on protein-coding genes in the human genome Nature 437, 1153-7 (2005).
Thomas, P. D. & Gilbert, D. Beyond Serendipity. The Scientist 16, 12 (2002).
Marth, G. et al. Single-nucleotide polymorphisms in the public domain: how useful are they? Nat Genet 27, 371-2 (2001).
De La Vega, F. M. et al. New generation pharmacogenomic tools: a SNP linkage disequilibrium Map, validated SNP assay resource, and high-throughput instrumentation system for large-scale genetic studies. Biotechniques Suppl, 48-50, 52, 54 (2002).
De La Vega, F. M. et al. The linkage disequilibrium maps of three human chromosomes across four populations reflect their demographic history and a common underlying recombination pattern. Genome Res 15, 454-62 (2005).
Reich, D. E., Gabriel, S. B. & Altshuler, D. Quality and completeness of SNP databases. Nat Genet 33, 457-8 (2003).
Pruitt, K. D. & Maglott, D. R. RefSeq and LocusLink: NCBI gene-centered resources. Nucleic Acids Res 29, 137-40 (2001).
Hubbard, T. et al. The Ensembl genome database project. Nucleic Acids Res 30, 38-41 (2002).
Tobler, A. R. et al. The SNPlex Genotyping System: A Flexible and Scalable Platform for SNP Genotyping. J. Biomolec. Tech. 16, 398-406 (2005).
Thomas, P. D. et al. PANTHER: a library of protein families and subfamilies indexed by function. Genome Res 13, 2129-41 (2003).
Cho, R. J. & Campbell, M. J. Transcription, genomes, function. Trends Genet 16, 409-15 (2000).

Thomas, P. D. & Kejariwal, A. Coding single-nucleotide polymorphisms associated with complex vs. Mendelian disease: evolutionary evidence for differences in molecular effects. Proc Natl Acad Sci USA 101, 15398-403 (2004).

### SUPPLEMENTAL REFERENCES FOR IN SILICO PROTEIN ANALYSIS

Wu, C. H. et al. The Universal Protein Resource (UniProt): an expanding universe of protein information. Nucleic Acids Res 34, D187-91 (2006).
Birney, E. et al. Ensembl 2006. Nucleic Acids Res 34, D556-61 (2006).
Finn, R. D. et al. Pfam: clans, web tools and services. Nucleic Acids Res 34, D247-51 (2006).
Kouranov, A. et al. The RCSB PDB information portal for structural genomics. Nucleic Acids Res 34, D302-5 (2006).
Andreeva, A. et al. SCOP database in 2004: refinements integrate structure and sequence family data. Nucleic Acids Res 32, D226-9 (2004).
Edgar, R. C. MUSCLE: multiple sequence alignment with high accuracy and high throughput. Nucleic Acids Res 32, 1792-7 (2004).
Orlicky, S., Tang, X., Willems, A., Tyers, M. & Sicheri, F. Structural basis for phosphodependent substrate selection and orientation by the SCFCdc4 ubiquitin ligase. Cell 112, 243-56 (2003).
Cerna, D. & Wilson, D. K. The structure of Sif2p, a WD repeat protein functioning in the SET3 corepressor complex. J Mol Biol 351, 923-35 (2005).
Sprague, E. R., Redd, M. J., Johnson, A. D. & Wolberger, C. Structure of the C-terminal domain of Tup1, a corepressor of transcription in yeast. Embo J 19, 3016-27 (2000).

Pickles, L. M., Roe, S. M., Hemingway, E. J., Stifani, S. & Pearl, L. H. Crystal structure of the C-terminal WD40 repeat domain of the human Groucho/TLE1 transcriptional corepressor. Structure 10, 751-61 (2002).
Li, D. & Roberts, R. WD-repeat proteins: structure characteristics, biological function, and their involvement in human diseases. Cell Mol Life Sci 58, 2085-97 (2001).
Smith, T. F., Gaitatzes, C., Saxena, K. & Neer, E. J. The WD repeat: a common architecture for diverse functions. Trends Biochem Sci 24, 181-5 (1999).
Ye, Y. & Godzik, A. FATCAT: a web server for flexible structure comparison and structure similarity searching. Nucleic Acids Res 32, W582-5 (2004).
Nicholas, K., Nicholas, H. & Deerfield, D. GeneDoc: Analysis and visualization of genetic variation. EMBNEW. NEWS 4, 14 (1997).
Clamp, M., Cuff, J., Searle, S. M. & Barton, G. J. The Jalview Java alignment editor. Bioinformatics 20, 426-7 (2004).
Galter, N., Gouy, M. & Gautier, C. SEAVIEW and PHYLO_WIN: two graphic tools for sequence alignment and molecular phylogeny. Comput Appl Biosci 12, 543-8 (1996).
Kabsch, W. & Sander, C. Dictionary of protein secondary structure: pattern recognition of hydrogen-bonded and geometrical features. Biopolymers 22, 2577-637 (1983).
McGuffin, L. J., Bryson, K. & Jones, D. T. The PSIPRED protein structure prediction server. Bioinformatics 16, 404-5 (2000).
Pyo, J. O. et al. Essential roles of Atg5 and FADD in autophagic cell death: dissection of autophagic cell death into vacuole formation and cell death. J Biol Chem 280, 20722-9 (2005).
Rosl, B., Yachdav, G. & Liu, J. The Predict Protein server. Nucleic Acids Res 32, W321-6 (2004).
Albrecht, M., Tosatto, S. C., Lengauer, T. & Valle, G. Simple consensus procedures are effective and sufficient in secondary structure prediction. Protein Eng 16, 459-62 (2003).
Bujnicki, J. M., Elofsson, A., Fischer, D. & Rychlewski, L. Structure prediction meta server. Bioinformatics 17, 750-1 (2001).
Jaroszewski, L., Rychlewski, L., Li, Z., Li, W. & Godzik, A. FFAS03: a server for profile-profile sequence alignments. Nucleic Acids Res 33, W284-8 (2005).
von Öhsen, N., Sommer, I., Zimmer, R. & Lengauer, T. Arby: automatic protein structure prediction using profile-profile alignment and confidence measures. Bioinformatics 20, 2228-35 (2004).
Ginalski, K. & Rychlewski, L. Detection of reliable and unexpected protein fold predictions using 3D-Jury. Nucleic Acids Res 31, 3291-2 (2003).
Appleton, B. A., Wu, P. & Wiesmann, C. The crystal structure of murine coronin-1: a regulator of actin cytoskeletal dynamics in lymphocytes. Structure 14, 87-96 (2006).
Rodriguez, R., Chinea, G., Lopez, N., Pons, T. & Vriend, G. Homology modeling, model and software evaluation: three related resources. Bioinformatics 14, 523-8 (1998).
Rybakin, V. & Clemen, C. S. Coronin proteins as multifunctional regulators of the cytoskeleton and membrane trafficking. Bioessays 27, 625-632 (2005).

### BOOKS

Abbas A K, Litchman A H. Cellular and Molecular Immunology. Philadelphia: Saunders; 1994. 417 p.
Austen B M and Westwood O M R. Protein Targeting and Secretion. Oxford: IRL Press; 1991.85 p.
Bishop M J, editor. Guide to Human Genome Computing, 2d ed. San Diego: Academic Press; 1998. 306 p.
Cowell I G, Austin C A, editors. DNA Library Protocols. Methods in Molecular Biology. Vol. 69 Totowa, N.J.: Humana Press; 1997. 321 p.
Freshney R I, editor. Animal Cell Culture: A Practical Approach. Oxford: IRL Press; 1986.
Freshney R I. Culture Of Animal Cells: A Manual of Basic Technique. New York: AR Liss; 1987. 397 p.
Glover D M, editor. DNA Cloning: A Pratical Approach. Vols 1 & 2. Oxford; Washington: IRL Press; 1985.
Gribskov M, Devereux J, editors. Sequence Analysis Primer. Oxford University Press; 1994. 296 p.
Griffin A M, Griffin H G, editors. Computer Analysis of Sequence Data, Part 1. Totowa, N.J. Humana Press; 1994. 392 p.
Hames B D, Higgins S J, editors. Nucleic Acid Hybridization: A Practical Approach. Oxford: IRL Press; 1985. 245 p.
Hames B D, Higgins S J, editors. Transcription and Translation: A Practical Approach. Oxford: IRL Press; 1984. 328 p.
Harlow Ed, Lane D. Antibodies: A Laboratory Manual. New York: Cold Spring Harbor Laboratory; 1988. 726 p.
Heinje G. von. Sequence Analysis in Molecular Biology. San Diego: Academic Press; 1987. 188 p.

Hogan B, Costantini F, Lacy E, editors. Manipulating the Mouse Embryo: A Laboratory Manual. New York: Cold Spring Harbor Laboratory Press; 1986. 332 p.
Huber B E, Carr B I. Molecular and Immunologic Approaches. Mt. Kisco, N.Y.: Futura Publishing Co; 1994.
Jones J. Amino Acid and Peptide Synthesis. Oxford; New York: Oxford Science Publications; 1992. 86 p.
Kaufman P B, William W, Donghern K, editors. Handbook of Molecular and Cellular Methods in Biology and Medicine. Boca Raton: CRC Press; 1995. 484 p.
Lesk A M, editor. Computational Molecular Biology: sources and methods for sequence analysis. New York: Oxford University Press; 1988. 254 p.
Male D, Cooke A, Owen M, Trowsdale J, Champion B, editors. Advanced Immunology. 3rd ed. London; Baltimore: Mosby; 1996. 273 p.
McPherson M J, editor. Directed Mutagenesis: A Practical Approach. New York: IRL Press; 1991. 257 p.
McPherson M J, Quirke P, Taylor J R, editors. PCR: A Practical Approach. Oxford; New York: IRL Press; 1991. 253 p.
Miller J H, Calos M P, editors. Gene Transfer Vectors for Mammalian Cells. New York: Cold Spring Harbor Laboratory Press; 1987. 169 p.
Miller J H, Calos M P, editors. Gene Transfer Vectors For Mammalian Cells. New York: Cold Spring Harbor Laboratory; 1987. 169 p.
Pawlowitzki I H, Edwards J H, Thompson E A, editors. Genetic Mapping of Disease Genes. Academic Press London; 1997. 288 p.
Perbal B V. A Practical Guide to Molecular Cloning. 1st ed. New York: Wiley Interscience Publication; 1984. 554 p.

Perbal B V. A Practical Guide To Molecular Cloning. New York: Wiley; 1984. 554 p.
Peruski L F, Peruski A H. The Internet and the New Biology. Tools for Genomic and Molecular Research. Washington, D.C.: American Society for Microbiology Press; 1997.
Sambrook J. Molecular Cloning: A Laboratory Manual. 2nd ed, 3 vols. New York; Cold Spring Harbor Laboratory Press; 1989.
Sell S. Immunology, Immunopathology & Immunity. 5th ed. Stamford, Conn.: Appleton & Lange; 1996. 1014 p.
Smith D W, editor. Biocomputing. Informatics and Genome Projects, New York: Academic Press; 1993. 336 p.
Stites D P, Terr A T, editors. Basic and Clinical Immunology. 7th ed. Norwalk, Conn.: Appleton & Lange; 1991. 870 p.
Walker J M. Protein Protocols on Crohn disease-ROM, Humana Press, Totowa, N.J.

Weir D M, Herzenberg L A, Blackwell C, editors. Handbook Of Experimental Immunology. 4 vols. Oxford: Blackwell; 1986.
Woodward J. Immobilized Cells And Enzymes: A Practical Approach. Oxford: IRL Press; 1986.
Wu R, Grossman L, editors. Methods in Enzymology: Rexcombinant DNA Part E. Vol. 154. Amsterdam: Elsevier Science; 1987. 576 p.
Wu R, Grossman L, editors. Methods in Enzymology: Rexcombinant DNA Part F. Vol. 155. Amsterdam: Elsevier Science; 1987. 628 p.

### PATENTS

U.S. Pat. No. 4,683,202.
U.S. Pat. No. 4,952,501.
W003042661A2
US 20040009479A1
U.S. Pat. No. 5,315,000
W01997US0005216
U.S. Pat. No. 5,498,531
U.S. Pat. No. 5,807,718
U.S. Pat. No. 5,888,819
U.S. Pat. No. 6,090,543
U.S. Pat. No. 6,090,606
U.S. Pat. No. 5,585,089
U.S. Pat. No. 4,683,195
U.S. Pat. No. 4,683,202
U.S. Pat. No. 5,459,039.
U.S. Pat. No. 6,090,543).
U.S. Pat. No. 6,090,606
U.S. Pat. No. 5,869,242
U.S. 60/335,068
U.S. Pat. No. 6,479,244
PCT/US94/05700
U.S. Pat. No. 4,797,368
WO 93/24641
U.S. Pat. No. 5,173,414

TABLE 1
Crohn disease candidate regions identified from the genome
wide scan association analyses in the QFP. The first column
denotes the region identifier. The second and third columns
correspond to the chromosome and cytogenetic band,
respectively. The fourth and fifth columns correspond to the
chromosomal start and end coordinates of the NCBI genome
assembly derived from build 35 (B35).

| Region | Chromosome | Cytogenetic Band | B35 Start | B35 End |
|---|---|---|---|---|
| 1 | 2 | 2q37.1 | 233464306 | 234464305 |

TABLE 2

| | | | | | | | | | | | | | | | | Single Type | |
|---|---|---|---|---|---|---|---|---|---|---|---|---|---|---|---|---|---|
| | | | | | | LDSTATS v2 | | | | | LDSTATS v4 | | | | | Single | |
| | | | | | | | | | | | | | | | | Genotype | |
| Region | | B35 | | Seq | | Single | | | | | Single | | | | | Likelihood | Single Allele |
| ID | Chr | Position | RS# | ID | Flanking Sequence | Marker | W03 | W05 | W07 | W09 | Marker | W03 | W05 | W07 | W09 | Ratio | Likelihood Ratio |
| 1 | 2 | 233466588 | 737027 | 223 | TAGCTCTGTTSCTATTTGCCA | 0.282 | 0.054 | 0.031 | 1.278 | 1.103 | 0.263 | 0.010 | 0.025 | 1.277 | 0.083 | 0.134 | 0.253 |
| 1 | 2 | 233505149 | 1867778 | 224 | AAAAGTGGTGYTAGAAAACTT | 0.182 | 0.152 | 0.008 | 0.194 | 2.506 | 0.155 | 0.070 | 0.001 | 0.188 | 2.277 | 0.082 | 0.219 |
| 1 | 2 | 233515765 | 2344614 | 225 | ATGTGTCAGCRGTTGCTTCTT | — | — | — | — | — | — | — | — | — | — | — | — |
| 1 | 2 | 233521207 | 7587309 | 226 | GTGTTTACCAKGTAGTGTGCT | 0.182 | 0.031 | 0.152 | 0.575 | 0.476 | 0.169 | 0.028 | 0.153 | 0.580 | 0.479 | 0.081 | 0.182 |
| 1 | 2 | 223544313 | 2044449 | 227 | TAAGATACACRTGAAGTCAAT | — | — | — | — | — | — | — | — | — | — | — | — |
| 1 | 2 | 233956749 | 809639 | 228 | ATAACTGATASGTTTTGAGAA | 0.196 | 0.035 | 0.201 | 0.590 | 0.564 | 0.179 | 0.017 | 0.213 | 0.596 | 0.574 | 0.268 | 0.196 |
| 1 | 2 | 233606587 | 2344912 | 229 | AAGAAGTAGCRGAGCTGAGAA | — | — | — | — | — | — | — | — | — | — | — | — |
| 1 | 2 | 233617614 | 2676960 | 230 | ACCGGGTGATKCATTTCCACA | — | — | — | — | — | — | — | — | — | — | — | — |
| 1 | 2 | 233635802 | 4973560 | 231 | GGTGCCAGCCRCCACCAGAAG | 0.397 | 0.853 | 0.265 | 0.131 | 0.467 | 0.395 | 0.874 | 0.233 | 0.138 | 0.451 | 0.598 | 0.397 |
| 1 | 2 | 233644264 | 4973583 | 232 | TCCTATTGGCRTGAGGATAAC | — | — | — | — | — | — | — | — | — | — | — | — |
| 1 | 2 | 233661159 | 7583124 | 233 | AAAACCATAAYGATTGGTGTT | 1.177 | 0.326 | 0.112 | 0.109 | 0.211 | 1.076 | 0.328 | 0.092 | 0.117 | 0.212 | 0.802 | 1.177 |
| 1 | 2 | 233671260 | 4973591 | 234 | TCTCGCTATCRTCTTCTGCCT | 0.107 | 0.365 | 0.150 | 0.197 | 0.259 | 0.094 | 0.375 | 0.154 | 0.205 | 0.239 | 0.147 | 0.107 |
| 1 | 2 | 233687921 | 5437082 | 235 | CACTGCTTCCMGCCACCCTGC | — | — | — | — | — | — | — | — | — | — | — | — |
| 1 | 2 | 233691534 | 684089 | 236 | ATAAGCCTCTSCCTTCTGGAA | — | — | — | — | — | — | — | — | — | — | — | — |
| 1 | 2 | 233713472 | 5437084 | 237 | AAGCGGGGTGYGATGTTTGGA | 0.000 | 0.124 | 0.228 | 0.307 | 0.061 | 0.000 | 0.134 | 0.260 | 0.300 | 0.066 | 0.287 | 0.000 |
| 1 | 2 | 233739452 | 4405750 | 238 | CAGGGCAGGGYTTGGGGCTGG | — | — | — | — | — | — | — | — | — | — | — | — |
| 1 | 2 | 233751966 | 5437087 | 239 | GGTTAAGTGAKTGACAACAGT | — | — | — | — | — | — | — | — | — | — | — | — |
| 1 | 2 | 233760103 | 4973055 | 240 | TGTTTTTAAGRCTCTTGATAC | 0.822 | 0.258 | 0.144 | 0.137 | 0.484 | 0.772 | 0.248 | 0.151 | 0.126 | 0.452 | 0.566 | 0.822 |
| 1 | 2 | 233776237 | 7608422 | 241 | CTGGGGAATGRGATGCAAGTG | 0.392 | 0.251 | 0.112 | 0.392 | 0.097 | 0.345 | 0.224 | 0.107 | 0.423 | 0.099 | 0.168 | 0.391 |
| 1 | 2 | 233788755 | 6437097 | 242 | CCTCCTATAAMATCCACTCCT | 0.276 | 0.336 | 0.072 | 0.127 | 0.009 | 0.240 | 0.342 | 0.087 | 0.125 | 0.096 | 0.345 | 0.276 |
| 1 | 2 | 233803717 | 6605277 | 243 | TTAAGAACAMACATTTTGGA | — | — | — | — | — | — | — | — | — | — | — | — |
| 1 | 2 | 233803717 | 7605743 | 244 | AGGCCCCATGKTTCCACCTGC | — | — | — | — | — | — | — | — | — | — | — | — |
| 1 | 2 | 233626871 | 6756920 | 245 | GTACCAACTAYCTGCAAGGCA | 1.054 | 0.419 | 0.074 | 0.065 | 0.109 | 0.972 | 0.426 | 0.082 | 0.076 | 0.194 | 0.674 | 0.154 |
| 1 | 2 | 233858566 | 7581787 | 246 | TTCTTTACGAMCACTGAAATT | — | — | — | — | — | — | — | — | — | — | — | — |
| 1 | 2 | 233862263 | 6431239 | 247 | GAGAGTGGCCRGTCCTAGATG | 0.669 | 0.494 | 0.331 | 0.084 | 0.206 | 0.628 | 0.497 | 0.310 | 0.090 | 2.220 | 0.668 | 0.669 |
| 1 | 2 | 233883168 | 3924334 | 248 | TGGTATTAGAWGAAACACATT | — | — | — | — | — | — | — | — | — | — | — | — |
| 1 | 2 | 233697739 | 14243 | 249 | AACACTCATGRTGTGCCAAGT | 0.068 | 0.473 | 1.179 | 0.356 | 0.031 | 0.049 | 0.465 | 1.151 | 0.355 | 0.03. | 0.024 | 0.109 |
| 1 | 2 | 233905010 | 7580869 | 250 | CCCCAGAAACRGACTCTGAGT | 0.649 | 1.136 | 0.442 | 0.480 | 0.242 | 0.609 | 1.129 | 0.463 | 0.472 | 0.260 | 0.384 | 0.649 |
| 1 | 2 | 233922610 | 7564252 | 251 | TCTCTTTTGGTGAGAAATGGA | — | — | — | — | — | — | — | — | — | — | — | — |
| 1 | 2 | 233949234 | 6431660 | 252 | ATTGAAAACTRAAAACATTTC | 2.602 | 1.385 | 0.600 | 1.061 | 0.808 | 2.396 | 1.383 | 0.598 | 1.067 | 0.780 | 2.000 | 2.570 |
| 1 | 2 | 233962638 | 3792110 | 253 | TACTTTGACCWGGTTTAACTT | — | — | — | — | — | — | — | — | — | — | — | — |
| 1 | 2 | 233966113 | 6861 | 254 | AGGAGTCAGGYGGCCTTCCCA | 0.467 | 1.059 | 0.651 | 0.865 | 0.744 | 0.442 | 1.154 | 0.665 | 0.847 | 0.775 | 0.414 | 0.439 |
| 1 | 2 | 234002157 | 6431282 | 255 | ACCCTGGAGCYGGCCTCCTCT | 0.662 | 0.521 | 0.821 | 0.453 | 2.825 | 0.589 | 0.543 | 0.790 | 0.480 | 0.817 | 0.354 | 0.662 |
| 1 | 2 | 234037548 | 1046976 | 256 | AAGAATGACGYTGATGAGTGA | — | — | — | — | — | — | — | — | — | — | — | — |
| 1 | 2 | 234046846 | 1550532 | 257 | ATATTTGAACSTTGCGTCTAG | 0.214 | 0.406 | 0.269 | 0.712 | 0.849 | 0.189 | 0.874 | 0.275 | 0.736 | 0.849 | 0.605 | 0.243 |
| 1 | 2 | 234066744 | 838709 | 258 | AACCACAGAGMGCATGTGTGT | 0.304 | 0.307 | 0.427 | 0.344 | 0.676 | 0.289 | 0.317 | 0.433 | 0.313 | 0.681 | 0.588 | 0.304 |
| 1 | 2 | 234075861 | 4663580 | 259 | TGGAGACTTGYGCTTCCCCCC | — | — | — | — | — | — | — | — | — | — | — | — |
| 1 | 2 | 234103752 | 638732 | 260 | CTTTAAAAAAYAGAGAGTCAG | 0.605 | 0.108 | 0.116 | 0.196 | 0.273 | 0.565 | 0.112 | 0.123 | 0.228 | 0.265 | 0.407 | 0.605 |
| 1 | 2 | 234142638 | 2228938 | 261 | CTGGTTCCTTGCCCGGTGGCT | — | — | — | — | — | — | — | — | — | — | — | — |
| 1 | 2 | 234169621 | 2971664 | 262 | CTGGGGAACTRTTGGATGTCT | 0.287 | 0.157 | 0.022 | 0.029 | 0.148 | 0.241 | 0.150 | 0.025 | 0.030 | 0.149 | 0.143 | 0.287 |
| 1 | 2 | 234207819 | 6431482 | 263 | TCAGTAGAATRGCCATGTTGG | — | — | — | — | — | — | — | — | — | — | — | — |
| 1 | 2 | 234222393 | 6716988 | 264 | AAAATGATGGYTATTCTCATT | 0.287 | 0.039 | 0.037 | 0.063 | 0.028 | 0.246 | 0.038 | 0.030 | 0.072 | 0.024 | 0.144 | 0.308 |
| 1 | 2 | 234244619 | 4663699 | 265 | TGCCAGAGTGWGGAAGAACAT | 0.210 | 0.107 | 0.049 | 0.044 | 0.047 | 0.180 | 0.035 | 0.055 | 0.044 | 0.048 | 0.074 | 0.210 |
| 1 | 2 | 234250148 | 6720619 | 266 | TCCATGTAATMGGTGTTGTAT | 0.287 | 0.053 | 0.038 | 0.251 | 0.088 | 0.261 | 0.039 | 0.032 | 0.267 | 0.081 | 0.139 | 0.273 |
| 1 | 2 | 234275046 | 4129945 | 267 | ACAGTCATCTRCCAGTGCGCC | 0.368 | 0.154 | 0.290 | 0.070 | 0.115 | 0.296 | 0.127 | 0.305 | 0.068 | 0.133 | 0.078 | 0.366 |
| 1 | 2 | 234287285 | 1115381 | 268 | CATGTGGAGCYGTGAGTATCT | — | — | — | — | — | — | — | — | — | — | — | — |
| 1 | 2 | 234300011 | 2741027 | 269 | TTAGTACCTGRTACAGATTCA | 0.064 | 0.371 | 0.236 | 0.126 | 0.032 | 0.075 | 0.377 | 0.229 | 0.142 | 0.025 | 0.059 | 0.084 |
| 1 | 2 | 234311122 | 1551265 | 270 | GATATAAAAAMGTATATATGA | — | — | — | — | — | — | — | — | — | — | — | — |
| 1 | 2 | 234318637 | 1377460 | 271 | GAGAGTATAARTGTTATATCA | 0.8090 | 0.407 | 0.191 | 0.035 | 0.222 | 0.790 | 0.393 | 0.199 | 0.044 | 0.230 | 0.454 | 0.809 |
| 1 | 2 | 234365062 | 2602379 | 272 | TCCATTAATTRTAGTAACAGG | — | — | — | — | — | — | — | — | — | — | — | — |
| 1 | 2 | 234378214 | 6754100 | 273 | AGAATTCAAGMCCATACATTC | 0.019 | 0.303 | 0.119 | 0.299 | 0.307 | 0.000 | 0.288 | 0.125 | 0.255 | 0.317 | 0.003 | 0.034 |
| 1 | 2 | 234388162 | 6715829 | 274 | TTTTTTTTTTWAAAAACTTTT | — | — | — | — | — | — | — | — | — | — | — | — |
| 1 | 2 | 234405117 | 4663945 | 275 | ATTGTAATAGRAGAATGTTTC | 0.334 | 0.045 | 0.452 | 0.383 | 0.256 | 0.270 | 0.028 | 0.478 | 0.406 | 0.248 | 0.477 | 0.334 |
| 1 | 2 | 234417467 | 4294999 | 276 | CCTCTATTCGRCCATTTAAAT | — | — | — | — | — | — | — | — | — | — | — | — |
| 1 | 2 | 234455242 | 11563252 | 277 | TCCAGATGAGYTTCAGTGTAA | — | — | — | — | — | — | — | — | — | — | — | — |

Results from the Crohn's Disease genome wide association study using the Quebec Founder Population (QFP) for 1 associated region.
Individual SNP markers genotyped in the genome wide scan are presented in each row of the table. The corresponding chromosome region ID is presented as identified in Tabel 1.
The chromosome number and coordinate of the SNP according to the NCBI genome assembly build 35 are indicated in columns 2 and 3.
The RS# column corresponds to the NCBI dbSNP identifier for the SNP. The Seq ID is the unique numerical identifier for this SNP in the sequence listing for this patent.
The column labeld Flanking Sequence corresponds to 21 bp of nucleotide sequence centerd at the SNP, which is coded using the standard degenerate naming system.
The remainder of the table lists-log 10 p values for association of the indicated haplotype centered at the corresponding SNP with the disease as described in the text, using LDSTATS V2, V4 and Single Type. Values for the association of single markers, as well as 3, 5, 7 and 9 marker haplotype windows are shown [see EXAMPLE 1 section for explanation of statistical calculations].

TABLE 3
List of associated haplotypes based on the Crohn Disease genome wide association study (GWAS)
using the Quebec Founder Population (QFP). Individual haplotypes with their relative risks (RR) are
presented in each row of the table: these data were extracted from the associated marker haplotype
window with the most significant p value for each SNP in Table 2. The first column lists the region ID
as presented in Table 1. The Haplotype column lists the specific nucleotides for the individual SNP
alleles contributing to the haplotype reported.
The Case and Control columns correspond to the numbers of case and control chromosomes, respectively,
containing the haplotype variant noted in the Haplotype column.
The Total Case and Total Control columns list the total numbers of case and control chromosomes for which
genotype data was available for the haplotype in question. The RR column corresponds to the odds ratio for
the haplotype.
The remainder of the columns lists the SeqIDs for the SNPs contributing to the haplotype and their relative
location with respect to the central marker.
The Central marker (0) column lists the SeqID for the central marker on which the haplotype is based.
Flaniding markers are identified by minus (−) or plus (+) signs to indicate the relative location of flanking SNPs.
See Table 2 for additional information on the central SNP of the haplotype.

| | | | | | | | Central | Central | Central | Central | Central | Central | Central | Central | Central |
|---|---|---|---|---|---|---|---|---|---|---|---|---|---|---|---|
| Region | | | Con- | Total | Total | | marker | marker | marker | marker | marker | marker | marker | marker | marker |
| ID | Haplotype | Case | trol | Case | Control | RR | (−4) | (−3) | (−2) | (−1) | (0) | (+1) | (+2) | (+3) | (+4) |
| 1 | CTTGGTA | 26 | 46 | 754 | 754 | 0.550 | | 223 | 224 | 226 | 228 | 231 | 233 | 234 | |
| 1 | CTTGGTG | 44 | 67 | 754 | 754 | 0.635 | | 223 | 224 | 226 | 228 | 231 | 233 | 234 | |
| 1 | GTTGACGTA | 3 | 11 | 754 | 754 | 0.270 | 223 | 224 | 226 | 228 | 231 | 233 | 234 | 237 | 240 |
| 1 | CTTGGTGCG | 33 | 58 | 754 | 754 | 0.549 | 223 | 224 | 226 | 228 | 231 | 233 | 234 | 237 | 240 |
| 1 | GTTGACATG | 31 | 14 | 754 | 754 | 2.266 | 223 | 224 | 226 | 228 | 231 | 233 | 234 | 237 | 240 |
| 1 | TTGGTGCGG | 54 | 78 | 752 | 752 | 0.669 | 224 | 226 | 228 | 231 | 233 | 234 | 237 | 240 | 241 |
| 1 | TGGTGCGGC | 43 | 65 | 752 | 752 | 0.641 | 226 | 228 | 231 | 233 | 234 | 237 | 240 | 241 | 242 |
| 1 | TGACATGGA | 22 | 9 | 752 | 752 | 2.488 | 226 | 228 | 231 | 233 | 234 | 237 | 240 | 241 | 242 |
| 1 | ACATGGA | 23 | 11 | 752 | 752 | 2.125 | | 231 | 233 | 234 | 237 | 240 | 241 | 242 | |
| 1 | ACGCGGA | 1 | 8 | 752 | 752 | 0.124 | | 231 | 233 | 234 | 237 | 240 | 241 | 242 | |
| 1 | GCGGCCA | 7 | 18 | 752 | 752 | 0.383 | | 234 | 237 | 240 | 241 | 242 | 245 | 247 | |
| 1 | CGACTAAGG | 20 | 8 | 752 | 752 | 2.541 | 237 | 240 | 241 | 242 | 245 | 247 | 249 | 250 | 252 |
| 1 | TGACTAAGA | 1 | 8 | 752 | 752 | 0.124 | 237 | 240 | 241 | 242 | 245 | 247 | 249 | 250 | 252 |
| 1 | GGCTAAGGC | 95 | 66 | 752 | 752 | 1.503 | 240 | 241 | 242 | 245 | 247 | 249 | 250 | 252 | 254 |
| 1 | TAAGG | 169 | 120 | 754 | 754 | 1.526 | | | 245 | 247 | 249 | 250 | 252 | | |
| 1 | CAAGA | 1 | 8 | 754 | 754 | 0.124 | | | 245 | 247 | 249 | 250 | 252 | | |
| 1 | CTAAGGCCG | 72 | 41 | 754 | 754 | 1.836 | 242 | 245 | 247 | 249 | 250 | 252 | 254 | 255 | 257 |
| 1 | TAAGGCCGA | 84 | 51 | 754 | 754 | 1.726 | 245 | 247 | 249 | 250 | 252 | 254 | 255 | 257 | 258 |
| 1 | AAGGCCGAT | 50 | 23 | 754 | 754 | 2.257 | 247 | 249 | 250 | 252 | 254 | 255 | 257 | 258 | 260 |
| 1 | GAGACCGAC | 2 | 11 | 754 | 754 | 0.180 | 247 | 249 | 250 | 252 | 254 | 255 | 257 | 258 | 260 |
| 1 | AAGCCCCTA | 4 | 13 | 754 | 754 | 0.304 | 249 | 250 | 252 | 254 | 255 | 257 | 258 | 260 | 262 |
| 1 | AGGCCGATG | 15 | 5 | 754 | 754 | 3.041 | 249 | 250 | 252 | 254 | 255 | 257 | 258 | 260 | 262 |
| 1 | GGCCGATGT | 20 | 5 | 754 | 754 | 4.082 | 250 | 252 | 254 | 255 | 257 | 258 | 260 | 262 | 264 |
| 1 | GCCGATGTA | 25 | 7 | 754 | 754 | 3.660 | 252 | 254 | 255 | 257 | 258 | 260 | 262 | 264 | 265 |
| 1 | CCGATGTAC | 31 | 14 | 754 | 754 | 2.266 | 254 | 255 | 257 | 258 | 260 | 262 | 264 | 265 | 266 |
| 1 | ACACTAAGA | 34 | 56 | 754 | 754 | 0.589 | 258 | 260 | 262 | 264 | 266 | 266 | 267 | 269 | 271 |
| 1 | ACTAAGAAG | 53 | 76 | 754 | 754 | 0.674 | 262 | 264 | 265 | 266 | 267 | 269 | 271 | 273 | 275 |

TABLE 4
List of candidate genes from the regions identified from the Genome Wide association analysis from
the QFP, derived from B35. The first column corresponds to the region identifier provided in Table 1.
The second and third columns correspond to the chromosome and cytogenetic band, respectively.
The fourth and fifth columns correspond to the chromosomal start and end coordinates of the NCBI
genome assembly derived from build 35 (B35, the start and end position relate to the + orientation
of the NCBI assembly and do not necessarily correspond to the orientation of the gene). The sixth and
seventh columns correspond to the official gene symbol and gene name, respectively,
and were obtained from the NCBI Entrez Gene database. The eighth column corresponds to the NCBI
Entrez Gene Identifier (GeneID). The ninth and tenth columns correspond to the Sequence IDs from
nucleotide (cDNA) and protein entries in the Sequence Listing.

| | Chro- | Cyto- | Start | | | | Entrez | Nucle- | |
|---|---|---|---|---|---|---|---|---|---|
| Region | mo- | genetic | Position | End position | Gene | Gene | Gene | otide | Protein |
| ID | some | Band | B35 | B35 | Symbol | Name | ID | Seq ID | Seq ID |
| 1 | 2 | 2q37.1 | 233387546 | 223548823 | TNRC15 | trinucleotide repeat containing 15 | 26058 | 1 | 2 |
| 1 | 2 | 2q37 | 233456679 | 233466780 | KCNJ13 | potassium inwardly-rectifying channel, subfamily J, | 3769 | 3 | 4 |
| | | | | | | member 13 | | | |
| 1 | 2 | 2q37.1 | 233560499 | 233566612 | UNQ830 | ASCL830 | 389084 | 5 | 6 |
| 1 | 2 | 2q37 | 233568920 | 233703443 | NGEF | neuronal guanine nucleotide exchange factor | 25791 | 7 | 8 |
| 1 | 2 | 2q37 | 233722887 | 233725272 | NEU2 | sialidase 2 (cytosolic sialidase) | 4759 | 9 | 10 |
| 1 | 2 | 2q36- | 233851676 | 233898543 | INPP5D | inositol polyphosphate-5-phosphatase, 145 kDa | 3635 | 11 | 12 |
| | | q37 | | | | | | | |
| 1 | 2 | 2q37.1 | 233942300 | 233985315 | ATG16L1 | ATG16 autophagy related 16-like 1 (S. cerevisiae) | 55054 | 13, 15, | 14, 16, |
| | | | | | | | | 17 | 18 |
| 1 | 2 | 2q37.1 | 233998493 | 234037701 | SAG | S-antigen; retina and pineal gland (arrestin) | 6295 | 19 | 20 |
| 1 | 2 | 2q37.1 | 234045153 | 234162743 | DGKD | diacylglycerol kinase, delta 130 kDa | 8527 | 21, 23 | 22, 24 |
| 1 | 2 | 2q37.1 | 234168038 | 234251869 | USP40 | ubiquitin specific peptidase 40 | 55230 | 25 | 26 |
| 1 | 2 | 2q37 | 234276085 | 234276937 | UGT1A12P | UDP glucuronosyltransferase 1 family, polypeptide | 54573 | — | — |
| | | | | | | A12 pseudogene | | | |
| 1 | 2 | 2q37 | 234294199 | 234295044 | UGT1A11P | UDP glucuronosyltransferase 1 family, polypeptide | 54574 | — | — |
| | | | | | | A11 pseudogene | | | |
| 1 | 2 | 2q37 | 234308291 | 234463945 | UGT1A8 | UDP glucuronosyltransferase 1 family, polypeptide | 54576 | 27 | 28 |
| | | | | | | A8 | | | |
| 1 | 2 | 2q37 | 234327123 | 234463951 | UGT1A10 | UDP glucuronosyltransferase 1 family, polypeptide | 54575 | 29 | 30 |
| | | | | | | A10 | | | |
| 1 | 2 | 2q37 | 234338575 | 234339672 | UGT1A13P | UDP glucuronosyltransferase 1 family, polypeptide | 404204 | — | — |
| | | | | | | A13 pseudogene | | | |
| 1 | 2 | 2q37 | 234362544 | 234463951 | UGT1A9 | UDP glucuronosyltransferase 1 family, polypeptide | 54600 | 31 | 32 |
| | | | | | | A9 | | | |
| 1 | 2 | 2q37 | 234372584 | 234463945 | UGT1A7 | UDP glucuronosyltransferase 1 family, polypeptide | 54577 | 33 | 34 |
| | | | | | | A7 | | | |
| 1 | 2 | 2q37 | 234382321 | 234463945 | UGT1A6 | UDP glucuronosyltransferase 1 family, polypeptide | 54578 | 35, 37 | 36, 38 |
| | | | | | | A6 | | | |
| 1 | 2 | 2q37 | 234403636 | 234463945 | UGT1A5 | UDP glucuronosyltransferase 1 family, polypeptide | 54579 | 39 | 40 |
| | | | | | | A5 | | | |
| 1 | 2 | 2q37 | 234409438 | 234463945 | UGT1A4 | UDP glucuronosyltransferase 1 family, polypeptide | 54657 | 41 | 42 |
| | | | | | | A4 | | | |
| 1 | 2 | 2q37 | 234419773 | 234463945 | UGT1A3 | UDP glucuronosyltransferase 1 family, polypeptide | 54659 | 43 | 44 |
| | | | | | | A3 | | | |
| 1 | 2 | 2q37 | 234437754 | 234439186 | UGT1A2P | UDP glucuronosyltransferase 1 family, polypeptide | 54580 | — | — |
| | | | | | | A2 pseudogene | | | |
| 1 | 2 | 2q37 | 234450919 | 234463945 | UGT1A1 | UDP glucuronosyltransferase 1 family, polypeptide | 54658 | 45 | 46 |
| | | | | | | A1 | | | |

TABLE 5
List of additional Crohn's disease candidate genes from the Genome Wide association analysis on
the QFP, derived from B36. In order to identify genes not placed in the regions from Table 1 according
to Build 35, the region coordinates were converted to Build 36 using the UCSC (University of California
Santa Cruz) online program LiftOver. Only new genes that were mapped to this version of the genome
asembly are included in this table. The first column corresponds to the region identifier provided in Table 1.
The second and third columns correspond to the chromosome and cytogenetic band, respectively. The
fourth and fifth columns correspond to the chromosomal start and end coordinates of the NCBI genome
assembly derived from build 36 (the start and end position relate to the + orientation of the NCBI
assembly and do not necessarily correspond to the orientation of the gene). The sixth and seventh columns
correspond to the official gene symbol and gene name, respectively, and were obtained from the NCBI
Entrez Gene database. The eighth column corresponds to the NCBI Entrez Gene Identifier (GeneID).
The ninth and tenth columns correspond to the Sequence IDs from nucleotide (cDNA) and protein entries
in the Sequence Listing.

| | | | Start | | | | | | |
|---|---|---|---|---|---|---|---|---|---|
| Region | | Cytogenetic | Position | End position | Gene | | Entrez | Nucleotide | Protein |
| ID | Chromosome | Band | B36 | B36 | Symbol | Gene Name | Gene ID | Seq ID | Seq ID |
| 1 | 2 | 2q37.1 | 233632820 | 233703606 | LOC653796 | similar to SH2 containing inositol | 653796 | 47 | 48 |
| | | | | | | phosphatase isoform b | | | |
| 1 | 2 | 2q37.1 | 233875714 | 233881802 | LOC642292 | hypothetical protein LOC642292 | 642292 | — | — |

TABLE 6
List of candidate genes based on EST clustering from the regions identified from the Genome
Wide association analysis on the QFP samples. The first column corresponds to the region
identifier provided in Table 1. The second column corresponds to the chromosome number.
The third and fourth columns correspond to the chromosomal start and end coordinates of the
NCBI genome assembly derived from build 35 (B35). The fifth column corresponds to the
ECGene identifier, corresponding to the ECGene track of UCSC. These ECGene entries
were determined by their overlap with the regions from Table 1, based on the start and end
coordinates of both Region and ECGene identifiers. The sixth and seventh columns correspond
to the Sequence IDs from nucleotide and protein entries in the Sequence Listing.

| | | Start Position | End Position | | Nucleotide | Protein |
|---|---|---|---|---|---|---|
| Region ID | Chromosome | Build35 | Build35 | Name | Seq ID | Seq ID |
| 1 | 2 | 233387530 | 233548841 | H2C23833.3 | 49 | 50 |
| 1 | 2 | 233387530 | 233550792 | H2C23833.4 | 51 | 52 |
| 1 | 2 | 233387544 | 233510194 | H2C23833.6 | 53 | 54 |
| 1 | 2 | 233387544 | 233548841 | H2C23833.7 | 66 | 56 |
| 1 | 2 | 233387545 | 233548841 | H2C23833.8 | 57 | 58 |
| 1 | 2 | 233387545 | 233550792 | H2C23833.9 | 59 | 60 |
| 1 | 2 | 233387552 | 233548841 | H2C23833.10 | 61 | 62 |
| 1 | 2 | 233387552 | 233550792 | H2C23833.11 | 63 | 64 |
| 1 | 2 | 233456565 | 233466780 | H2C23854.1 | 65 | 66 |
| 1 | 2 | 233456565 | 233466780 | H2C23854.2 | 67 | 68 |
| 1 | 2 | 233456679 | 233466780 | H2C23854.3 | 69 | 70 |
| 1 | 2 | 233456679 | 233466780 | H2C23854.4 | 71 | 72 |
| 1 | 2 | 233458355 | 233466780 | H2C23854.5 | 73 | 74 |
| 1 | 2 | 233500007 | 233548841 | H2C23833.18 | 75 | 76 |
| 1 | 2 | 233559846 | 233566616 | H2C23881.1 | 77 | 78 |
| 1 | 2 | 233565640 | 233566616 | H2C23881.2 | 79 | 80 |
| 1 | 2 | 233565994 | 233566621 | H2C23881.3 | 81 | 82 |
| 1 | 2 | 233565994 | 233568017 | H2C23881.4 | 83 | 84 |
| 1 | 2 | 233565994 | 233568923 | H2C23881.5 | 85 | 86 |
| 1 | 2 | 233566735 | 233568923 | H2C23833.19 | 87 | 88 |
| 1 | 2 | 233568900 | 233571840 | H2C23883.1 | 89 | 90 |
| 1 | 2 | 233568900 | 233581180 | H2C23883.2 | 91 | 92 |
| 1 | 2 | 233568900 | 233703448 | H2C23883.3 | 93 | 94 |
| 1 | 2 | 233660266 | 233665011 | H2C23883.4 | 85 | 96 |
| 1 | 2 | 233702828 | 233706098 | H2C23890.1 | 97 | 98 |
| 1 | 2 | 233702975 | 233706099 | H2C23890.2 | 99 | 100 |
| 1 | 2 | 233722886 | 233725272 | H2C23891.1 | 101 | 102 |
| 1 | 2 | 233750074 | 233898546 | H2C23892.1 | 103 | 104 |
| 1 | 2 | 233750074 | 233898867 | H2C23892.2 | 105 | 106 |
| 1 | 2 | 233763379 | 233769546 | H2C23895.1 | 107 | 108 |
| 1 | 2 | 233851680 | 233898867 | H2C23908.1 | 109 | 110 |
| 1 | 2 | 233894424 | 233896298 | H2C23926.1 | 111 | 112 |
| 1 | 2 | 233897478 | 233898548 | H2C23930.1 | 113 | 114 |
| 1 | 2 | 233942270 | 233986319 | H2C23935.1 | 119 | 120 |
| 1 | 2 | 233942270 | 233986319 | H2C23935.2 | 115 | 116 |
| 1 | 2 | 233942270 | 233986319 | H2C23935.3 | 117 | 118 |
| 1 | 2 | 233942270 | 233986319 | H2C23935.4 | 121 | 122 |
| 1 | 2 | 233942270 | 233986672 | H2C23935.5 | 125 | 126 |
| 1 | 2 | 233942270 | 233986672 | H2C23935.6 | 123 | 124 |
| 1 | 2 | 233942270 | 233986672 | H2C23935.7 | 127 | 128 |
| 1 | 2 | 233942299 | 233986319 | H2C23935.8 | 129 | 130 |
| 1 | 2 | 233942303 | 233974103 | H2C23935.9 | 131 | 132 |
| 1 | 2 | 233942317 | 233986312 | H2C23935.10 | 133 | 134 |
| 1 | 2 | 233964366 | 233986319 | H2C23835.11 | 135 | 136 |
| 1 | 2 | 233996169 | 233997039 | H2C23951.1 | 137 | 138 |
| 1 | 2 | 233998461 | 234037701 | H2C23953.1 | 139 | 140 |
| 1 | 2 | 233998481 | 234037701 | H2C23953.2 | 141 | 142 |
| 1 | 2 | 233998461 | 234037701 | H2C23953.3 | 143 | 144 |
| 1 | 2 | 233998461 | 234037701 | H2C23953.4 | 145 | 146 |
| 1 | 2 | 233998466 | 234009650 | H2C23953.5 | 147 | 148 |
| 1 | 2 | 233998485 | 234017978 | H2C23953.6 | 149 | 150 |
| 1 | 2 | 234018458 | 234036600 | H2C23953.7 | 151 | 152 |
| 1 | 2 | 234027657 | 234037701 | H2C2396.1 | 153 | 154 |
| 1 | 2 | 234028706 | 234037701 | H2C23953.8 | 155 | 156 |
| 1 | 2 | 234045152 | 234162746 | H2C23965.1 | 157 | 158 |
| 1 | 2 | 234045219 | 234083043 | H2C23965.2 | 159 | 160 |
| 1 | 2 | 234078799 | 234162746 | H2C23965.3 | 161 | 162 |
| 1 | 2 | 234089131 | 234096394 | H2C23973.1 | 163 | 164 |
| 1 | 2 | 234148884 | 234150513 | H2C23965.4 | 165 | 166 |
| 1 | 2 | 234154459 | 234157796 | H2C23965.5 | 167 | 168 |
| 1 | 2 | 234166164 | 234180286 | H2C23997.1 | 169 | 170 |
| 1 | 2 | 234166164 | 234251867 | H2C239973.2 | 171 | 172 |
| 1 | 2 | 234176254 | 234177022 | H2C23997.3 | 173 | 174 |
| 1 | 2 | 234183046 | 234203148 | H2C23997.4 | 175 | 176 |
| 1 | 2 | 234244970 | 234251867 | H2C23997.5 | 177 | 178 |
| 1 | 2 | 234251390 | 234251867 | H2C23997.6 | 179 | 180 |
| 1 | 2 | 234264392 | 234268480 | H2C240101.1 | 181 | 182 |
| 1 | 2 | 234308290 | 234463956 | H2C24012.1 | 183 | 184 |
| 1 | 2 | 234327099 | 234463956 | H2C24012.2 | 185 | 186 |
| 1 | 2 | 234327119 | 234460918 | H2C24012.3 | 187 | 188 |
| 1 | 2 | 234362498 | 234463956 | H2C24012.4 | 189 | 190 |
| 1 | 2 | 234372583 | 234463956 | H2C24012.5 | 191 | 192 |
| 1 | 2 | 234373013 | 234382992 | H2C24019.1 | 193 | 194 |
| 1 | 2 | 234382252 | 234460918 | H2C24012.6 | 195 | 196 |
| 1 | 2 | 234382252 | 234463956 | H2C24012.7 | 197 | 198 |
| 1 | 2 | 234382347 | 234447020 | H2C24012.8 | 199 | 200 |
| 1 | 2 | 234383511 | 234399340 | H2C24020.1 | 201 | 202 |
| 1 | 2 | 234383511 | 234463956 | H2C24012.9 | 203 | 204 |
| 1 | 2 | 234383532 | 234420610 | H2C24012.10 | 205 | 206 |
| 1 | 2 | 234383532 | 234460918 | H2C24012.11 | 207 | 208 |
| 1 | 2 | 234403637 | 234408719 | H2C24012.12 | 209 | 210 |
| 1 | 2 | 234403637 | 234463956 | H2C24012.13 | 211 | 212 |
| 1 | 2 | 234409423 | 234463956 | H2C24012.14 | 213 | 214 |
| 1 | 2 | 234419753 | 234463956 | H2C24012.15 | 215 | 216 |
| 1 | 2 | 234444951 | 234445915 | H2C24028.1 | 217 | 218 |
| 1 | 2 | 234444951 | 234445991 | H2C24026.2 | 219 | 220 |
| 1 | 2 | 234450891 | 234463956 | H2C24012.16 | 221 | 222 |

TABLE 7
Top 72 CD-associated SNPs, ranked with respect to the p-value obtained in an allele-based case-control comparison
(CCA) in panel A. Also included are the p-values for the genotype-based case-control comparison (CCG) and the TDT.
Nucleotide positions refer to NCBI build 34. Markers with p < 0.05 in either the case-control or the TDT analysis in
replication panel B are highlighted in bold italics. SNPs with a significant result in both panel B tests are additionally
marked by grey shading. In addition to rs2241880, only SNP rs1050152 (Leu503Phe) in the SLC22A4 gene, reported
earlier by Pettekova et al. and the known CARD15 SNP rs2066845 (“SNP12) yielded consistent replication.

| | | | Screening (panel A) | | | | | Replication (panel B) | | |
|---|---|---|---|---|---|---|---|---|---|---|
| # | Gene | Celera ID | dbSNP ID | chr. | position | P_{CCA} | P_{CCG} | P_{CCA} | P_{CCG} | P_{TDT} |
| 1 | DCP1B | hCV2194128 | rs12423058 | 12 | 1,934,927 | 5.8 * 10⁻¹⁴ | 3.6 * 10⁻¹³ | 0.92 | 0.54 | 0.07 |
| 2 | TINAG | hCV1202797 | rs1058768 | 6 | 54,232,983 | 1.7 * 10⁻¹² | 7.9 * 10⁻¹¹ | 0.27 | 0.15 | 0.21 |
| | | 2 | | | | | | | | |
| 3 | OR8H1 | hCV2577077 | rs17613241 | 11 | 55,839,523 | 2.2 * 10⁻⁰⁹ | 2.8 * 10⁻⁰⁹ | 0.27 | 0.41 | 0.15 |
| | | 5 | | | | | | | | |
| 4 | TTN | hCV2562648 | rs10497517 | 2 | 179,646,084 | 3.4 * 10⁻⁰⁷ | 2.7 * 10⁻⁰⁶ | 0.17 | 0.37 | 0.7 |
| | | 8 | | | | | | | | |
| 5 | OR10A4 | hCV1589535 | rs2595453 | 11 | 6,862,804 | 0.00005 | 0.0003 | 0.18 | 0.21 | 0.43 |
| | | 2 | | | | | | | | |
| 6 | hCG17440 | hCV3111449 | rs211716 | 1 | 75,529,932 | 0.0001 | 0.0006 | 0.05 | 0.16 | 0.14 |
| | 77 | | | | | | | | | |
| 7 | hCG17440 | hCV928121 | rs211715 | 1 | 75,530,066 | 0.0002 | 0.001 | 0.07 | 0.21 | 0.19 |
| | 77 | | | | | | | | | |
| [symbol] | [symbol] | [symbol] | [symbol] | [symbol] | [symbol] | [symbol] | [symbol] | [symbol] | [symbol] | [symbol] |
| 9 | IL7R | hCV2025977 | rs6897932 | 5 | 35,920,076 | 0.0004 | 0.0002 | 0.91 | 0.99 | 0.95 |
| [symbol] | [symbol] | [symbol] | [symbol] | [symbol] | [symbol] | [symbol] | [symbol] | [symbol] | [symbol] | [symbol] |
| 11 | FLJ23577 | hCV2577012 | — | 5 | 35,715,804 | 0.0004 | 0.004 | 0.21 | 0.41 | 0.81 |
| | | 3 | | | | | | | | |
| 12 | U2 | hCV2563797 | rs6730351 | 2 | 223,793,960 | 0.0007 | 0.003 | 0.55 | 0.81 | 0.5 |
| | | 5 | | | | | | | | |
| [symbol] | [symbol] | [symbol] | [symbol] | [symbol] | [symbol] | [symbol] | [symbol] | [symbol] | [symbol] | [symbol] |
| 14 | [symbol] | hCV1911085 | rs1165165 | 6 | 25,970,445 | 0.0009 | 0.004 | 0.58 | 0.26 | 0.9 |
| [symbol] | [symbol] | [symbol] | [symbol] | [symbol] | [symbol] | [symbol] | [symbol] | [symbol] | [symbol] | [symbol] |
| | [symbol] | [symbol] | [symbol] | | | | | | | |
| 16 | NALP13 | hCV2092168 | rs303997 | 19 | 61,116,255 | 0.001 | 0.005 | 0.73 | 0.86 | 0.82 |
| 17 | hCG18121 | hCV2599494 | rs10483261 | 14 | 20,346,679 | 0.001 | 0.005 | 0.79 | 0.76 | 0.08 |
| | 62 | 2 | | | | | | | | |
| 18 | hCG16464 | hCV1596554 | rs2291479 | 3 | 179,495,857 | 0.001 | 0.006 | 0.49 | 0.72 | 0.6 |
| | 71 | 5 | | | | | | | | |
| 19 | HS6ST3 | hCV3118872 | rs2282135 | 13 | 95,187,906 | 0.001 | 0.003 | 0.12 | 0.25 | 0.86 |
| [symbol] | [symbol] | [symbol] | [symbol] | [symbol] | [symbol] | [symbol] | [symbol] | [symbol] | [symbol] | [symbol] |
| 21 | VGF | hCV2564960 | — | 7 | 100,378,082 | 0.002 | 0.006 | 0.27 | 0.17 | 0.13 |
| | | 9 | | | | | | | | |
| [symbol] | [symbol] | [symbol] | [symbol] | [symbol] | [symbol] | [symbol] | [symbol] | [symbol] | [symbol] | [symbol] |
| 23 | PLSCR4 | hCV2564738 | rs3762685 | 3 | 147,259,528 | 0.002 | 0.005 | 0.71 | 0.77 | 0.3 |
| | | 3 | | | | | | | | |
| 24 | OR5U1 | hCV2519378 | rs9257694 | 6 | 29,382,496 | 0.002 | 0.008 | 0.52 | 0.77 | 1 |
| [symbol] | [symbol] | [symbol] | [symbol] | [symbol] | [symbol] | [symbol] | [symbol] | [symbol] | [symbol] | [symbol] |
| | | [symbol] | | | | | | | | |
| [symbol] | [symbol] | [symbol] | [symbol] | [symbol] | [symbol] | [symbol] | [symbol] | [symbol] | [symbol] | [symbol] |
| | | [symbol] | | | | | | | | |
| 27 | FUCA1 | hCV1202362 | rs11549094 | 1 | 23,650,437 | 0.002 | 0.008 | 0.48 | 0.54 | 0.69 |
| | | 9 | | | | | | | | |
| 28 | hCG19995 | hCV2481084 | rs3129096 | 6 | 29,291,365 | 0.002 | 0.008 | 0.45 | 0.67 | 0.83 |
| | 32 | | | | | | | | | |
| 29 | OR2J2 | hCV1119478 | rs3116817 | 6 | 29,257,553 | 0.002 | 0.01 | 0.57 | 0.84 | 0.61 |
| | | 3 | | | | | | | | |
| 30 | FLJ25660 | hCV2537241 | rs541169 | 19 | 40,410,860 | 0.003 | 0.01 | 0.77 | 0.39 | 0.32 |
| 31 | KUB3 | hCV2577032 | rs3751325 | 12 | 56,621,893 | 0.003 | 0.001 | 0.46 | 0.75 | 0.25 |
| | | 0 | | | | | | | | |
| 32 | SLC16A4 | hCV1596127 | rs2271885 | 1 | 110,220,442 | 0.003 | 0.01 | 0.31 | 0.09 | 0.1 |
| | | 5 | | | | | | | | |
| [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] |
| | | [symbol] | | | | | | | | |
| 36 | DHX34 | hCV1150706 | rs12984558 | 19 | 52,548,176 | 0.003 | 0.01 | 0.82 | 0.87 | 0.36 |
| | | 4 | | | | | | | | |
| 37 | hCG26636 | hCV2942610 | rs1864147 | 16 | 64,719,751 | 0.003 | 0.01 | 0.06 | 0.16 | 0.29 |
| 38 | DP58 | hCV622249 | rs32857 | 5 | 79,939,445 | 0.003 | 0.0008 | 0.93 | 0.36 | 0.15 |
| 39 | PLSCR4 | hCV9539784 | rs1061409 | 3 | 147,238,670 | 0.003 | 0.01 | 0.93 | 0.99 | 0.29 |
| [symbol] | [symbol] | [symbol] | [symbol] | [symbol] | [symbol] | [symbol] | [symbol] | [symbol] | [symbol] | [symbol] |
| | [symbol] | | | | | | | | | |
| 41 | ST5 | hCV1506057 | rs3812762 | 11 | 8,715,949 | 0.003 | 0.008 | 0.71 | 0.54 | 0.87 |
| 42 | OAS2 | hCV8920052 | rs15895 | 12 | 111,860,241 | 0.004 | 0.01 | 0.6 | 0.74 | 0.91 |
| 43 | FLJ46906 | hCV8275411 | rs1129180 | 6 | 138,998,702 | 0.004 | 0.006 | 0.72 | 0.91 | 0.51 |
| 44 | C14orf125 | hCV8601135 | rs7157977 | 14 | 29,848,248 | 0.004 | 0.02 | 0.29 | 0.35 | 0.96 |
| [symbol] | [symbol] | [symbol] | [symbol] | [symbol] | [symbol] | [symbol] | [symbol] | [symbol] | [symbol] | [symbol] |
| 46 | CACNA1E | hCV1432822 | rs704326 | 1 | 178,999,038 | 0.004 | 0.01 | 0.32 | 0.61 | 0.17 |
| 47 | KNSL7 | hCV2592411 | rs3804583 | 3 | 44,845,239 | 0.004 | 0.01 | 0.22 | 0.38 | 1 |
| | | 1 | | | | | | | | |
| [symbol] | [symbol] | [symbol] | [symbol] | [symbol] | [symbol] | [symbol] | [symbol] | [symbol] | [symbol] | [symbol] |
| | | [symbol] | | | | | | | | |
| 49 | SLC1A4 | hCV2681351 | rs759458 | 2 | 65,219,899 | 0.004 | 0.02 | 0.81 | 0.82 | 0.25 |
| 50 | U15 | hCV3215915 | rs4774310 | 15 | 56,701,220 | 0.005 | 0.02 | 0.23 | 0.45 | 0.33 |
| 51 | MYO10 | hCV3132500 | rs27431 | 5 | 16,723,356 | 0.005 | 0.01 | 0.7 | 0.83 | 0.55 |
| 52 | IFI44L | hCV1187369 | rs3820093 | 1 | 78,518,119 | 0.005 | 0.007 | 0.35 | 0.63 | 0.92 |
| | | 4 | | | | | | | | |
| 53 | CAPSL | hCV8811801 | rs1445898 | 5 | 35,956,030 | 0.005 | 0.01 | 0.35 | 0.53 | 0.75 |
| 54 | FLJ31846 | hCV2595981 | rs3764147 | 13 | 42,255,925 | 0.005 | 0.009 | 0.05 | 0.11 | 0.77 |
| | | 1 | | | | | | | | |
| 55 | hCG17947 | hCV37420 | rs13092702 | 3 | 147,440,204 | 0.005 | 0.02 | 0.28 | 0.43 | 0.79 |
| | 90 | | | | | | | | | |
| [symbol] | [symbol] | [symbol] | [symbol] | [symbol] | [symbol] | [symbol] | [symbol] | [symbol] | [symbol] | [symbol] |
| | | [symbol] | | | | | | | | |
| 57 | NUDCD1 | hCV1588332 | rs2980618 | 8 | 110,258,581 | 0.006 | 0.02 | 0.29 | 0.56 | 0.33 |
| | | 9 | | | | | | | | |
| 58 | UBAP2 | hCV8778477 | rs1785506 | 9 | 34,007,106 | 0.006 | 0.02 | 0.1 | 0.21 | 0.96 |
| [symbol] | [symbol] | [symbol] | [symbol] | [symbol] | [symbol] | [symbol] | [symbol] | [symbol] | [symbol] | [symbol] |
| 60 | LRRK2 | hCV3215842 | rs3761863 | 12 | 39,044,919 | 0.007 | 0.02 | 0.6 | 0.8 | 0.06 |
| [symbol] | [symbol] | [symbol] | — | [symbol] | [symbol] | [symbol] | [symbol] | [symbol] | [symbol] | [symbol] |
| | | [symbol] | | | | | | | | |
| 62 | hCG19941 | hCV2441812 | rs2157650 | 8 | 17,715,645 | 0.007 | 0.0004 | 0.11 | 0.24 | 0.13 |
| | 24 | | | | | | | | | |
| 63 | hCG20402 | hCV2598877 | rs10427252 | 2 | 215,765,119 | 0.007 | 0.02 | 0.09 | 0.14 | 0.9 |
| | 72 | 3 | | | | | | | | |
| [symbol] | [symbol] | [symbol] | — | [symbol] | [symbol] | [symbol] | [symbol] | [symbol] | [symbol] | [symbol] |
| | | [symbol] | | | | | | | | |
| 65 | C14orf8 | hCV2434490 | rs9624 | 14 | 19,490,249 | 0.008 | 0.01 | 0.35 | 0.64 | 0.21 |
| 66 | U10 | hCV1201713 | rs1826619 | 10 | 31,005,500 | 0.008 | 0.02 | 0.21 | 0.41 | 0.37 |
| | | 5 | | | | | | | | |
| 67 | FLJ23577 | hCV2574280 | rs7710284 | 5 | 35,738,276 | 0.008 | 0.03 | 0.8 | 0.53 | 0.42 |
| | | 5 | | | | | | | | |
| 68 | NALP8 | hCV8110157 | rs306481 | 19 | 61,179,415 | 0.008 | 0.04 | 0.97 | 0.93 | 0.96 |
| 69 | U1 | hCV2708023 | rs4534436 | 1 | 119,108,148 | 0.008 | 0.02 | 0.21 | 0.16 | 0.31 |
| | | 0 | | | | | | | | |
| 70 | IGHMBP2 | hCV2547453 | rs17612126 | 11 | 68,481,034 | 0.009 | 0.01 | 0.98 | 0.92 | 0.27 |
| | | 0 | | | | | | | | |
| 71 | USP16 | hCV2870492 | rs2274802 | 21 | 29,330,540 | 0.009 | 0.04 | 0.52 | 0.18 | 0.11 |
| 72 | CLEC2D | hCV2599256 | rs3764022 | 12 | 9,724,791 | 0.009 | 0.007 | 0.82 | 0.8 | 0.14 |
| | | 9 | | | | | | | | |

TABLE 8
Fine mapping of the CD association signal at the ATG16L1 locus. The p
values obtained in panel B in allele-based (CCA) and genotype-based
(CCG) association analyses of the tagging and coding SNPs are shown.
The only coding SNP in ATG16L1 (rs2241880) is highlighted in bold
italics. MAF: minor allele frequency.

| SNP ID | Position (build 35 | MAF | p_{CCG} | p_{CCA} | p_{TDT} |
|---|---|---|---|---|---|
| rs6757418 | 233,909,321 | 0.16 | 0.56 | 0.95 | 0.42 |
| rs11674242 | 233,916,795 | 0.12 | 0.47 | 0.23 | 0.88 |
| rs2341565 | 233,917,138 | 0.15 | 0.57 | 0.29 | 0.58 |
| rs12471808 | 233,920,324 | 0.46 | 0.68 | 0.81 | 0.46 |
| rs12472651 | 233,920,522 | 0.41 | 0.67 | 0.36 | 0.03 |
| rs10211468 | 233,921,467 | 0.43 | 0.24 | 0.8 | 0.13 |
| rs11675235 | 233,922,554 | 0.13 | 0.69 | 0.43 | 0.76 |
| rs4663340 | 233,924,691 | 0.08 | 0.02 | 0.004 | 0.25 |
| rs7563345 | 233,925,244 | 0.34 | 0.04 | 0.02 | 0.002 |
| rs2083575 | 233,927,080 | 0.07 | 0.07 | 0.03 | 0.04 |
| rs13412102 | 233,927,971 | 0.41 | 0.004 | 0.001 | 0.006 |
| rs12471449 | 233,928,958 | 0.14 | 0.0005 | 0.0001 | 0.02 |
| rs11685932 | 233,948,322 | 0.33 | 0.11 | 0.04 | 0.008 |
| rs6431660 | 233,949,234 | 0.47 | 0.0001 | 0.00002 | 0.0001 |
| rs1441090 | 233,950,042 | 0.07 | 0.002 | 0.001 | 0.13 |
| rs13011156 | 233,953,812 | 0.05 | 0.77 | 0.61 | 0.83 |
| rs12105443 | 233,961,028 | 0.01 | 0.63 | 0.63 | 0.74 |
| rs3792110 | 233,962,638 | 0.28 | 0.23 | 0.09 | 0.006 |
| rs2289476 | 233,963,556 | 0.06 | 0.88 | 0.64 | 0.8 |
| rs2289472 | 233,964,240 | 0.47 | 0.00007 | 0.00001 | 0.00002 |
| [symbol] | [symbol] | [symbol] | [symbol] | [symbol] | [symbol] |
| rs2241879 | 233,965,468 | 0.47 | 0.0001 | 0.00002 | 0.00003 |
| rs7600743 | 233,971,359 | 0.06 | 0.21 | 0.16 | 0.61 |
| rs3792106 | 233,972,740 | 0.41 | 0.0003 | 0.00008 | 0.00005 |
| rs4663396 | 233,974,251 | 0.2 | 0.0006 | 0.0001 | 0.02 |
| rs7587051 | 233,976,755 | 0.34 | 0.09 | 0.04 | 0.008 |
| rs6748547 | 233,984,766 | 0.05 | 0.69 | 0.73 | 1 |
| rs6759896 | 233,992,972 | 0.41 | 0.06 | 0.02 | 0.007 |

TABLE 9
Results of a haplotype analysis of 9 SNPs at the ATG16L1 locus. SNPs included in the haplotype
analysis are marked by asterisks in FIG. 2, thereby showing their block assignment. All analyses were
carried out using either COCAPHASE or TDTPHASE. Non-synonymous SNP rs2241880 is highlighted in
bold and the risk allele underlined. Obviously, the sole risk haplotype (ACACAGGCG) is fully signified by
rs2241880 allele G; all other haplotypes are protective and carry allele A. This haplotype pattern strongly
suggests that rs2241880 is indeed the major risk variant at the ATG16L1 locus.

| | | | | p-value | | | | p-value |
|---|---|---|---|---|---|---|---|---|
| Haplotype | f_{cases} | f_{controls} | OR_{case-control} | COCAPHASE | f_{transmitted} | f_{non-transmitted} | OR_{TDT} | TDTPHASE |
| ACACAGGCG | 0.603 | 0.532 | 1.34 | 0.00002 | 0.535 | 0.285 | 2.87 | 0.0001 |
| ACGCTAACG | 0.254 | 0.283 | 0.86 | 0.0502 | 0.262 | 0.396 | 0.54 | 0.0164 |
| AGATAAATG | 0.047 | 0.069 | 0.67 | 0.0045 | 0.077 | 0.092 | 0.82 | 0.5462 |
| GGACAAATG | 0.052 | 0.069 | 0.74 | 0.042 | 0.081 | 0.139 | 0.55 | 0.0608 |
| ACGCAAGTA | 0.044 | 0.048 | 0.91 | 0.5685 | 0.035 | 0.073 | 0.46 | 0.0728 |
| ACACAAGTG | <0.01 | <0.01 | n.d. | n.d. | 0.012 | 0.015 | 0.8 | 0.705 |

TABLE 10
Analysis of the statistical interaction between
ATG16L1 SNP rs2241880 and CARD15 genotype, coded as
described in the Example section.

| affection | | CARD15 | | |
|---|---|---|---|---|
| status | ATG16L1 | dd | dD | DD |
| control | GG | 219 | 62 | 2 |
| | AG | 435 | 87 | 2 |
| | AA | 185 | 35 | 5 |
| CD | GG | 175 | 92 | 42 |
| | AG | 232 | 136 | 57 |
| | AA | 73 | 50 | 21 |
| odds ratio | GG | 2.03 | 1.04 | 5.00 |
| (95% CI) | | (1.43-2.88) | (0.59-1.84) | (0.76-41.05) |
| | AG | 1.35 | 1.09 | 6.79 |
| | | (0.98-1.87) | (0.64-1.88) | (1.04-55.16) |
| | AA | 1 | 1 | 1 |

1. A method of determining susceptibility to Crohn's disease in a subject, comprising determining the presence or absence of at least one SNP in a biological sample from said subject, wherein said SNP is listed in Table 7 and wherein the presence of said at least one SNP indicates susceptibility to Crohn's Disease.

2. The method of claim 1, wherein said at least one SNP is in a ATG16L1 gene.

3. The method of claim 2, wherein said at least one SNP in the ATG16L1 gene is rs2241880.

4. The method of claim 1, wherein the presence or absence of at least one further SNP in a CARD15 gene is determined.

5. (canceled)

6. The method of claim 1, wherein the at least one SNP is listed in Table 8.

7.-11. (canceled)

12. The method of claim 1, wherein said subject has symptoms of an inflammatory bowel disease

13. The method of claim 12, wherein said symptoms are selected from the group consisting of diarrhea, abdominal pain, fever, fatigue, rectal bleeding, weight loss, and combinations thereof.

14. (canceled)

15. The method of claim 1, wherein the presence or absence of the at least one SNP is determined with an assay selected from the group consisting of electrophoretic analysis, restriction length polymorphism analysis, sequence analysis and hybridization analysis

16.-22. (canceled)

23. A method of genetic mapping for detecting the association of at least one marker for Crohn's disease comprising a) obtaining a biological samples from a group of patients, b) screening for the presence or absence of an allele of at least one SNP or a group of SNPs from Table 7 within each biological sample, and c) evaluating whether said at least one SNP or a group of SNPs shows a statistically significant skewed genotype distribution between the group of patients compared to a group of controls.

24. The method of claim 23, wherein said biological sample is selected from the group consisting of hair, fluid, serum, tissue swab, buccal swab, saliva, mucus, urine, stools, spermatozoids, vaginal secretions, lymph, amniotic fluid, pleural liquid and tear.

25. The method of claim 23, wherein said groups of patients and controls are from a human population.

26. The method of claim 23, wherein said groups of patients and controls are recruited independently according to a specific phenotypic criteria.

27. The method of claim 23, wherein said screening is performed by an assay selected from the group consisting of allele-specific hybridization, oligonucleotide ligation, allele-specific elongation/ligation, allele-specific amplification, single-base extension, molecular inversion probe, invasive cleavage, selective termination, restriction length polymorphism, sequencing, SSCP, mismatch-cleaving, and denaturing gradient gel electrophoresis.

28. The method of claim 23, wherein said screening is carried out on each individual of a cohort at each of at least one SNP or a group of SNPs from Table 7.

29. (canceled)

30. The method of claim 23, wherein the genotype distribution is compared one SNP at a time.

31. The method of claim 23, wherein the genotype distribution is compared with a group of markers from Table 7 forming a haplotype.

32. The method of claim 23, wherein the genotype distribution is compared using the allelic frequencies between the group of patients and controls.

33.-47. (canceled)

48. A method for inducing a Crohn's disease-like state in a tissue or cell, comprising contacting the tissue or cell with at least one gene listed in Table 7 to induces a Crohn Disease-like state in said tissue or cell.

49. The method of claim 48, wherein said cell is selected from the group consisting of smooth muscle cell, neutrophil, T cell, mast cell, Crohn's disease CD4+ lymphocyte, monocyte, macrophage, dendritic cell, synovial cell, glial cell, villous intestinal cell, neutrophilic granulocyte, eosinophilic granulocyte, keratinocyte, lamina propria lymphocyte, intraepithelial lymphocyte and epithelial cell.

50. A method for screening drug candidates for treating Crohn's disease, comprising: a) contacting a cell induced by the method of claim 48 with a drug candidate for treating Crohn's disease; and b) assaying for a pro-inflammatory like state, such that an absence of the pro-inflammatory like state is indicative of the drug candidate being effective in treating Crohn's disease.

51.-54. (canceled)

55. A drug screening assay comprising a) administering a test compound to an animal having Crohn's disease or a cell composition isolated therefrom, and b) comparing the level of gene expression of a gene listed in Table 7 in the presence of the test compound with one or both of the levels of said gene expression in the absence of the test compound or in normal cells, wherein test compounds which cause the level of expression of a gene listed in Table 7 to approach normal are candidates for drugs to treat Crohn's disease.

56.-60. (canceled)

61. A method of diagnosing susceptibility to Crohn's disease in an individual, comprising screening for an at-risk haplotype of at least one SNP from Table 7, that is more frequently present in an individual susceptible to Crohn's disease compared to a control individual, wherein the presence of the at-risk haplotype increases risk of Crohn s disease in said individual.

62. The method of claim 61, wherein the risk increase is at least about 20%.

63.-64. (canceled)

65. The method of claim 62, wherein said screening comprises enzymatic amplification of nucleic acid from said individual or amplification using universal oligos on elongation/ligation products.

66. The method of claim 65, wherein the nucleic acid is DNA.

67. The method of claim 66, wherein the DNA is human DNA.

68. The method of claim 62, wherein said screening comprises a) obtaining material containing nucleic acid from the individual (b) amplifying said nucleic acid, and c) determining the presence or absence of an at-risk haplotype in said amplified nucleic acid.

69. The method of claim 68, wherein determining the presence of an at-risk haplotype is performed by an assay selected from the group consisting of electrophoretic analysis, restriction length polymorphism analysis, sequence analysis, and hybridization analysis.

70.-74. (canceled)

75. A method of diagnosing a susceptibility to Crohn's disease in an individual, comprising detecting an alteration in the expression or composition of a polypeptide encoded by a gene listed in Table 7 a sample from said individual, in comparison with the expression or composition of a polypeptide encoded by said gene in a control sample, wherein the presence of an alteration in expression or the composition of the polypeptide in the sample from said individual is indicative of a susceptibility to Crohn's disease.

76. The method of claim 75, wherein the alteration in the expression or composition of a polypeptide encoded by said gene comprises expression of a splicing variant polypeptide in a test sample that differs from a splicing variant polypeptide expressed in a control sample.

77.-138. (canceled)

139. A method for predicting the efficacy of a drug for treating Crohn's disease in a human patient, comprising a) obtaining a sample of cells from the patient, b) obtaining a set of genotypes from the sample, wherein the set of genotypes comprises genotypes of one or more polymorphic loci from Table 7, and c) comparing the set of genotypes of the sample with a set of genotypes associated with efficacy of the drug, wherein similarity between the set of genotypes of the sample and the set of genotypes associated with efficacy of the drug predicts the efficacy of the drug for treating Crohn's disease in the patient.

140. The method of claim 139, wherein the sample of cells is derived from a tissue selected from the group consisting of the scalp, GI track, muscle, sebaceous gland nerve, blood, dermis, epidermis and other skin cells, cutaneous surfaces, intertrigious areas, genitalia, vessels and endothelium.

141. The method of claim 140, wherein the cells are selected from the group consisting of melanocytes, hair follicle cells, muscle cells, nerve cells, keratinocytes, monocytes, neutrophils, langerhans cells, Crohn's disease CD4+, Crohn's disease CD8+ T cells and lymphocytes.

142. The method of claim 139, wherein the sample is obtained via biopsy.

143. The method of claim 139, wherein the set of genotypes from the sample comprises genotypes of at least two of the polymorphic loci listed in Table 7.

144. The method of claim 139, wherein the set of genotypes from the sample is obtained by hybridization to allele-specific oligonucleotides complementary to the polymorphic loci from Table 7, wherein said allele-specific oligonucleotides are contained on a microarray.

145. The method of claim 144, wherein the oligonucleotides comprise nucleic acid molecules at least 95% identical to SEQ ID from Table 7.

146. The method of claim 144, wherein the set of genotypes from the sample is obtained by sequencing said polymorphic loci in said sample.

147. The method of claim 144, wherein the drug is selected from the group consisting of symptom relievers and drugs for Crohn's disease.

148.-158. (canceled)

159. A method of assessing a patient's risk of having or developing Crohn's disease, said method comprising a) determining a genotype for at least one polymorphic locus from Table 7 in said patient; b) comparing said genotype of a) to a genotype for at least one polymorphic locus from Table 7 that is associated with Crohn's disease; and c) assessing the patient's risk of having or developing Crohn's disease, wherein said patient has a higher risk of having or developing Crohn's disease with respect to a control individual if the genotype for at least one polymorphic locus from Table 7 in said patient is the same as said genotype for at least one polymorphic locus from Table 7 that is associated with Crohn's disease.

160. The method of claim 4, wherein the at least one SNP in the CARD15 gene is rs2066845.

161. The method of claim 1, wherein the presence or absence of at least one SNP in the SLC22A4 gene is determined.

162. The method of claim 161, wherein said at least one SNP in the SLC22A4 gene is rs1050152.
